(19) United States
(12) Patent Application Publication
(10) Pub. No.: US 2014/0336159 A1
(43) Pub. Date: Nov. 13, 2014

(54) METHODS FOR TREATING AND DIAGNOSING RESPIRATORY TRACT INFECTIONS

(71) Applicant: PULMATRIX, INC., Lexington, MA (US)

(72) Inventors: Robert William Clarke, Medfield, MA (US); David L. Hava, Natick, MA (US); John P. Hanrahan, West Roxbury, MA (US); Wesley Hugh Dehaan, Chelmsford, MA (US); Paulette Wright Andreotta, Charlton, MA (US); Stephen P. Arold, Billerica, MA (US); Jennifer Kenyon Saunders, Boston, MA (US)

(21) Appl. No.: 14/350,235

(22) PCT Filed: Oct. 5, 2012
(86) PCT No.: PCT/US2012/059022

§ 371 Date: Apr. 7, 2014

Related U.S. Application Data
(60) Provisional application No. 61/544,400, filed on Oct. 7, 2011.
(60) Provisional application No. 61/550,081, filed on Oct. 21, 2011.
(60) Provisional application No. 61/584,001, filed on Jan. 6, 2012.
(60) Provisional application No. 61/605,013, filed on Feb. 29, 2012.
(60) Provisional application No. 61/607,936, filed on Mar. 7, 2012.
(60) Provisional application No. 61/648,960, filed on May 18, 2012.
(60) Provisional application No. 61/648,822, filed on May 18, 2012.

(51) Int. Cl. A61K 31/19; A61K 9/00; A61K 31/56; A61K 45/06

(52) U.S. Cl. CPC A61K 31/19; A61K 45/06; A61K 9/0078; A61K 9/0075; A61K 31/56
USPC 514/171; 514/557

(57) ABSTRACT

Described are methods of preventing, treating and diagnosing of a subject having a condition, such as, an inflammation or infection of the respiratory tract. Methods of treatment and prevention include administration of effective amounts of calcium salt formulations to a subject. Methods of diagnosing include the use of biomarkers and optionally the use of kits that can detect biomarkers. Further described are methods for modulating an immune response that include the modulation of Toll-like receptors.

### RELATED APPLICATIONS

This application claims the benefit of U.S. Provisional Application No. 61/544,400, filed on Oct. 7, 2011, U.S. Provisional Application No. 61/550,081, filed on Oct. 21, 2011, U.S. Provisional Application No. 61/584,001, filed on Jan. 6, 2012, U.S. Provisional Application No. 61/605,013, filed on Feb. 29, 2012, U.S. Provisional Application No. 61/607,936, filed on Mar. 7, 2012, U.S. Provisional Application No. 61/648,960, filed on May 18, 2012 and U.S. Provisional Application No. 61/648,822, filed on May 18, 2012 the entire teachings of these applications are incorporated herein by reference.

### GOVERNMENT SUPPORT

This invention was made with Government support under Grant No.: W911NF-10-1-0382 awarded by the U.S. Army Research Office (ARO) and the Defense Advanced Research Projects Agency (DARPA). The Government has certain rights in the invention.

### BACKGROUND OF THE INVENTION

Long-term use of currently available respiratory drugs is frequently accompanied by unwanted side-effects. For example, long term use of high doses of antibiotics in the treatment of chronic airway infection can be accompanied by the emergence of antibiotic-resistant bacterial flora, such as with inhaled aminoglycoside treatment of Pseudomonas in Cystic Fibrosis or macrolide antibiotic treatment of non-tuberculous mycobacteria. If such resistance develops, the bacterial burden in the airways is no longer optimally controlled, leading to greater airway inflammation and respiratory exacerbations and the need for more aggressive therapeutic regimens that are often associated with untoward side effects. Accordingly, there remains a need to provide better long-term treatment for respiratory diseases and improved diagnosis of inflammation, irritation and/or infection of the respiratory tract.

### SUMMARY OF THE INVENTION

Aspects of the invention relate to treatment regimens for respiratory diseases. Calcium ions provide several beneficial activities when administered to the respiratory tract, such as anti-infective activity, anti-inflammatory activity and increasing mucociliary clearance (MCC). It has now been discovered that these activities are related to the administered dose of calcium ion and that the activities can be selectively provided to patients. The activities are induced in patients on a dosage continuum. Low doses provide substantially none or very low levels of activity, mid doses provide anti-infective activity and/or anti-inflammatory activity, but substantially none or very little measurable increase in MCC; while high doses provide anti-infective activity, anti-inflammatory activity and increased MCC. The effects of the calcium ion that is dosed may have some variability among members of the population. However, dosing can be easily adjusted to provide the desired calcium ion-induced activities. By dosing calcium ions, alone or in combination with other therapeutic agents optimized therapy can be provided.

Treatment regimens comprising the use of calcium salts in defined dose ranges of calcium ions can address problems of pathogen resistance and other problems of long-term use of agents for the treatment of respiratory diseases. For example, existing anti-inflammatories can be immunosuppressive and may lead to increased bacterial burden or rate of infection. Existing anti-infective agents can eradicate infection but have no impact on persistent or chronic inflammation. Calcium ions on the other hand can provide multiple beneficial therapeutic effects and avoid side-effects associated with current therapies. Calcium salt formulations can comprise additional therapeutic agents or they can be combined in low-, mid-, or high calcium doses with additional therapeutic agents administered separately. Therapeutic agents can include any known, effective, approved and available agents for the treatment of respiratory diseases, e.g. mucoactive or mucolytic agents, surfactants, antibiotics, antivirals, antihistamines, cough suppressants, bronchodilators, anti-inflammatory agents, steroids, vaccines, adjuvants, expectorants, antifibrotic agents, macromolecules, etc. When co-formulated or co-administered with therapeutic agents, calcium salt formulations can promote or augment the activity of the therapeutic agents and/or to enable lowering their respective effective doses in disease management.

Aspects of the invention further relate to methods of diagnosing, of selecting a subject for therapy, and for monitoring the efficacy of treatment of an inflammation, infection and/or irritation of the respiratory tract in a subject. Preferably, the therapy comprises administering calcium ions (e.g. in the form of a salt) in specific desired doses described herein. For example, a patient may present with an inflammation, irritation and/or infection which may be diagnosed by the methods described herein. Based on the diagnosis, a physician may then decide on an appropriate therapy comprising administering calcium ion in a dose appropriate to treat the diagnosed condition. If desired, the therapy may further include administering one or more additional therapeutic agents.

Aspects of the invention further relate to methods for modulating Toll-like receptor (TLR) signaling. The methods comprise contacting a TLR-expressing cell with mono- or divalent metal cation or salts thereof in an amount sufficient to modulate TLR signaling, e.g. signaling through one or more of TLR1, TLR2, TLR3, TLR4, TLR5, TLR6, TLR7, TLR8, TLR9, TLR10, and combinations thereof.

### BRIEF DESCRIPTION OF THE FIGURES

FIG. 1A shows the dose ranges found to be efficacious across multiple preclinical efficacy models. The equivalent human lung dose in mg Ca²⁺ ion per kg of bodyweight for each preclinical model is calculated as the lung dose in mg Ca²⁺ ion per kg of bodyweight of the model animal. FIG. 1B shows the equivalent human dose ranges found to be efficacious across multiple preclinical models when translated by matching dose per lung mass. The equivalent human lung dose in mg Ca²⁺ ion/kg for each preclinical model is calculated from the mass of Ca²⁺ ion deposited in the lung of the model animal, scaled by the ratio of the lung masses of the two species. FIG. 1C shows the equivalent human dose ranges found to be efficacious across multiple preclinical models when translated by matching dose per lung surface area. The equivalent human lung dose in mg Ca²⁺ ion/kg for each preclinical model is calculated from the mass of Ca²⁺ ion deposited in the lung of the model animal, scaled by the ratio of the lung surface areas of the two species.

FIG. 2A and FIG. 2B show changes in airway surface liquid (ASL) height. Normal human bronchial epithelial (NHBE) cells were treated with nebulized solutions of a liquid calcium formulation (Formulation I, 30 microgram/cm² or 10 microgram/cm², respectively) over a 15 minute period (FIG. 2A) or with deposition of a calcium dry powder formulation (Formulation II) (FIG. 2B) and changes in ASL height were measured in real-time. Effects on ASL height for dry powder Formulation II (30 microgram/cm² or 10 microgram/cm², respectively), matched sodium chloride dry powder (NaCl DP), and leucine control dry powder (Leucine DP) were measured. FIG. 2C shows ASL height changes when human bronchial epithelial cells from a donor with cystic fibrosis (CF HBE) were treated with dry powder Formulation II deposited on the air-liquid interphase.

FIG. 3 shows mucociliary clearance (MCC) velocity of Formulation II in healthy sheep. FIG. 3A: Immediately following dosing with Formulation II (3 exposed doses: 0.25, 0.5 and 1 mg Ca²⁺ ion/kg animal) and vehicle (placebo) dry powder control acute mucociliary clearance (MCC) was measured for 60 minutes. For reference, clearance reported for hypertonic (7%) saline at 1 hour is indicated. FIG. 3B: Clearance is shown for Formulation II (2 exposed doses: 0.5 and 1 mg Ca²⁺ ion/kg animal), liquid 7% hypertonic saline, and dry powder vehicle (placebo) control over a period between 2 and 3 hours post dosing.

FIG. 4 shows central lung clearance in healthy ex-smoking human subjects with COPD. The mean retained dose of radioisotope over time from central lung (including large and conducting airways) over 120 minutes is shown for baseline clearance velocity (circles) and Formulation II-augmented clearance velocity (triangles) for a nominal human dose of 22 mg calcium ion.

FIG. 5 shows human sputum levels of the inflammatory mediators IL-8 (FIG. 5A), IL-6 (FIG. 5B), GM-CSF (FIG. 5C), and IL-1 beta (FIG. 5D) assessed by immunoassay. Mediator levels were compared pre- and post-treatment with a calcium salt containing dry powder Formulation II (with 5.5 mg calcium ion nominal human dose (predicted human lung dose: 0.041 mg calcium ion per kg bodyweight) and 11 mg calcium ion nominal human dose (predicted human lung dose: 0.082 mg calcium ion per kg bodyweight), respectively) in human subjects with COPD.

FIG. 6 shows inflammatory cell counts (total cells (FIG. 6A) and neutrophils (FIG. 6B)) in sputum in human subjects with COPD. Cell levels were compared pre-(D0) and post-(D2) treatment with calcium salt containing dry powder Formulation II (with 5.5 mg calcium ion nominal human dose (predicted human lung dose: 0.041 mg calcium ion per kg bodyweight) and 11 mg calcium ion nominal human dose (predicted human lung dose: 0.082 mg calcium ion per kg bodyweight), respectively).

FIG. 7 shows allergen-induced sputum cosinophilia in mild atopic asthmatic human subjects when treated with placebo or liquid calcium salt formulation, Formulation V.

FIG. 8 shows calcium-containing formulations inhibit the movement of bacterial pathogens across mucus mimetic: (FIG. 8A) K. pneumoniae, (FIG. 8B) S. pneumoniae, (FIG. 8C) P. aeruginosa, and (FIG. 8D) S. aureus. Mucus mimetic was treated topically with aerosol [saline (closed circles) or Formulation V: 0.12 M CaC₁₂ in 0.15 M NaCl (open circles)] and bacteria were added immediately post-treatment.

FIG. 9 shows calcium-containing formulations reduce the movement of (FIG. 9 A) influenza virus (Influenza A/WSN/33/1) and (FIG. 9 B) rhinovirus (Rv16) across mucus mimetic.

FIG. 10 shows calcium-containing formulations do not reduce movement of Der p 1 across sodium alginate mucus mimetic. Sodium alginate mucus mimetic was exposed to the indicated formulations and HDM extract containing Der p 1 protein was added immediately post exposure.

FIG. 11A shows inhibition of influenza virus infection by 1.29% CaCl₂ in 0.9% NaCl (Formulation V) with and without zanamivir. FIG. 11B shows inhibition of viral infection by dry powder formulations of zanamivir, calcium salt, or zanamivir and calcium salt.

FIG. 12 shows inflammatory cell counts for two infection models (FIG. 12A) and two models of inflammation (FIG. 12B). FIG. 12A: In a mouse model of rhinovirus infection, mice were treated with Formulation II 1 hour before and 4 hours after infection. Bronchoalveolar lavage was performed 24 hours after infection and inflammatory cells were quantified (left panel). In a mouse model of LPS Pseudomonas aeruginosa challenge, mice were treated with Formulation II 1 hour before and 4 hours after LPS challenge. Bronchoalveolar lavage was performed 24 hours after LPS challenge and inflammatory cells were quantified (right panel). FIG. 12B: In a 4-day tobacco smoke-(TS) exposure model mice were treated with Formulation II or p38 MAPK inhibitor (+) before TS exposure once a day. Bronchoalveolar lavage was performed 4 hours after the last TS exposure and inflammatory cells were quantified (left panel; *** indicates p<0.001). In a mouse model of ozone exposure, mice were treated with Formulation II or p38 MAPK inhibitor (+) 1 hour prior to ozone exposure. Bronchoalveolar lavage was performed 4 hours after ozone exposure and inflammatory cells were quantified (right panel). Control animals for each model were treated with a dry powder (DP) placebo (100% leucine). FIG. 12C: Ozone-exposed animals were treated with Formulation III ((A) exposed dose: 0.8 mg calcium ion/kg animal, (B) exposed dose: 2.3 mg calcium ion/kg animal) and Formulation IV ((C): exposed dose 2.8 mg calcium ion/kg animal) and inflammatory cells were quantified.

FIG. 13 shows a Venn diagram summarizing the irritation and infection gene signature and the overlapping genes representative of the inflammation gene signature for both upregulated (FIG. 13A) and downregulated (FIG. 13B) genes as determined for the tobacco smoke/irritation model and the rhinovirus/infection model.

FIG. 14 shows measurements of inflammatory cytokine secretion into the media of peritoneal macrophages (PEM) exposed to a 1 ng/ml dose of LPS, and treated with increasing (0, 10, 25, and 50 mM) calcium chloride. Protein concentration in cell culture supernatents were measured for KC (FIG. 14A), IL-6 (FIG. 14B), and TNF alpha (FIG. 14C).

FIG. 15 shows measurements of inflammatory cytokine expression of peritoneal macrophages (PEM) exposed to a 1 ng/ml dose of LPS, and treated with increasing (0, 10, 25, and 50 mM) calcium chloride. Gene expression was measured for KC (FIG. 15A), IL-6 (FIG. 15B), and TNF alpha (FIG. 15C). Data is presented as a fold-change with respect to the “media only” group.

FIG. 16 shows gene expression in LPS stimulated mouse peritoneal macrophages (PEM) for ENA78, GM-CSF, MIP-2, IP-10, and NRIP1.

FIG. 17A shows measurements of inflammatory cytosine gene expression by human macrophages isolated from healthy normal donors exposed to a 10 ng/ml dose of LPS, and treated with 10 or 25 mM calcium chloride. Protein concentration in cell culture supernatents were measured for IL-8, IL-6, TNF alpha and MIP-1 alpha. FIG. 17B shows measurements of inflammatory cytokine gene expression by human macrophages isolated from chronic obstructive pulmonary disease (COPD)-donor blood exposed to a 10 ng/ml dose of LPS, and treated with concentrations of calcium chloride ranging from 10 mM to 50 mM. Protein concentration in cell culture supernatents was measured for IL-8.

FIG. 18 shows measurements of effects of different monovalent and divalent salts on inflammatory cytokine secretion into the media by peritoneal macrophages (PEM) exposed to 10 ng/ml LPS in cell culture media, and treated with increasing (0, 5, 10, 25, and 50 mM) concentrations of calcium chloride, calcium lactate, magnesium chloride, and sodium chloride. Protein concentrations in cell culture supernatents were measured for KC (FIG. 18A) and IL-6 (FIG. 18 B) secretion. FIG. 18C and FIG. 18D show measurements of effects of the same monovalent and divalent salts on secretion of the corresponding human inflammatory cytokines, IL-8 (FIG. 18C) and IL-6 (FIG. 18D) into the media by human macrophages isolated from healthy normal blood.

FIG. 19 shows graphs of cytokine secretion for KC and TNF alpha by isolated murine macrophages exposed to either (FIG. 19A, B) S. pneumoniae (1×10⁷ CFU/ml) or (FIG. 19C, D) K. pneumoniae (1×10⁷ CFU/ml), and treated with increasing (0, 5, 10 and 25 mM) concentrations of calcium chloride.

FIG. 20A shows KC secretion (pg/ml) by macrophages exposed to 1 ng/ml of LPS and treated with increasing concentrations of the TRPV channel antagonist ruthenium red (1, 5, 10 and 20 micromolar) with and without 10 mM calcium chloride. FIG. 20B shows KC, TNF alpha, and IL-6 secretion of macrophages exposed to 10 ng/ml of LPS and treated with TRPV2 antagonist SKF96365 (5, 20 and 50 micromolar) with and without 10 mM calcium chloride.

### DETAILED DESCRIPTION

In a first aspect, the invention relates to treatment of respiratory diseases (including, e.g., chronic airway diseases and pulmonary diseases) and respiratory conditions (including acute conditions, such as, e.g., acute pathogenic infections, inflammations and irritations), especially respiratory diseases associated with airway inflammation and/or excess airway mucus, such as asthma, airway hyper-responsiveness, seasonal allergic allergy, bronchiectasis, chronic bronchitis, emphysema, chronic obstructive pulmonary disease (COPD), cystic fibrosis (CF) and the like. Certain aspects also relate to the prevention and treatment (e.g. attenuation of severity) of acute exacerbations (worsening of symptoms, especially respiratory symptoms) of respiratory diseases (e.g. chronic airway diseases and pulmonary diseases), including asthma, airway hyper-responsiveness, seasonal allergic allergy, bronchiectasis, chronic bronchitis, emphysema, chronic obstructive pulmonary disease, cystic fibrosis and the like. The methods for treatment and/or prevention of the aforementioned diseases and conditions comprise administering to the respiratory tract an effective amount of a formulation comprising one or more calcium salt(s). Depending on the disease or condition to be treated, the calcium salt formulations may be administered to the respiratory tract and specifically the lungs in different doses of calcium ions to achieve different desired outcomes. Of particular importance are treatment regimens for chronic diseases marked by airway inflammation, such as asthma, COPD, CF, bronchiectasis, and the like. Patients affected by these respiratory diseases are dependent on long-term administration of therapeutic agents to control and manage the symptoms of the disease, such as excess airway mucus, chronic inflammation, and airflow limitation, but also to prevent exacerbations (e.g. brought on by pathogenic respiratory infections or other inhaled respiratory insults from the environment, such as air pollution) that can lead to serious morbidity and even fatalities.

When mucus secretion and mucus clearance are not in balance, excessive airway mucus can result. This condition can be associated with impaired airway clearance (impaired mucociliary clearance) and/or mucus hypersecretion. Excess mucus, which can be very viscous, may accumulate in the airways. The presence of excess airway mucus in respiratory diseases such as cystic fibrosis, chronic obstructive pulmonary disease, bronchiectasis and the like is well established. Impaired mucociliary clearance can be the result of damaged or poorly functioning cilia, excess mucus production, abnormally thick and viscous mucus, collapsed or inspisated mucus (e.g. resulting from improper mucus hydration as occurs with CFTR channel mutations seen in cystic fibrosis patients), and the like. The pulmonary consequences of airway mucus and secretions which have not been cleared (e.g. excessive quantity, abnormal viscosity, or retained secretions) include increased pulmonary symptoms such as shortness of breath, exacerbations, hospitalization, sharply declining FEV₁ and, when severe, death.

The calcium formulations (e.g. liquid or dry powder formulations) useful in the methods of treatment described herein may be administered without additional therapeutic agents or may be administered together with or in addition to one or more therapeutic agents. These can include any known, effective, approved and available agents for the treatment of respiratory diseases, e.g. mucoactive or mucolytic agents, surfactants, antibiotics, antivirals, antihistamines, cough suppressants, bronchodilators, anti-inflammatory agents, steroids, vaccines, adjuvants, expectorants, antifibrotic agents, macromolecules, etc.

Calcium formulations useful in the methods of treatment described herein may be administered in specific dose ranges for calcium ion to the lung (lung dose) depending on the dosing regimen chosen and the desired therapeutic benefit.

For example, the calcium formulations may be administered in an amount effective to deliver to the lung calcium ion in an amount of 0.075 mg Ca²⁺ ion/kg bodyweight to about 1.25 mg Ca²⁺ ion/kg bodyweight, preferably in an amount of 0.075 mg Ca²⁺ ion/kg bodyweight to about 0.75 mg Ca²⁺ ion/kg bodyweight, in an amount of 0.1 mg Ca²⁺ ion/kg bodyweight to about 1.0 mg Ca²⁺ ion/kg bodyweight, or in an amount of 0.125 mg Ca²⁺ ion/kg bodyweight to about 1.25 mg Ca²⁺ ion/kg bodyweight. In one embodiment, the calcium formulations may be administered in an amount effective to deliver to the lung calcium ion in an amount of 0.075 mg Ca²⁺ ion/kg bodyweight to about 0.5 mg Ca²⁺ ion/kg bodyweight. Administration of calcium ion doses in an amount of 0.075 mg Ca²⁺ ion/kg bodyweight to about 1.25 mg Ca²⁺ ion/kg bodyweight are referred to herein as “high dose” calcium ion administration.

If desired, the calcium formulations may be administered in an amount effective to deliver to the lung calcium ion in an amount of 0.005 mg Ca²⁺ ion/kg bodyweight to about 0.2 mg Ca²⁺ ion/kg bodyweight, preferably in an amount of 0.005 mg Ca²⁺ ion/kg bodyweight to about 0.05 mg Ca²⁺ ion/kg bodyweight, in an amount of 0.01 mg Ca²⁺ ion/kg bodyweight to about 0.1 mg Ca²⁺ ion/kg bodyweight, or in an amount of 0.02 mg Ca²⁺ ion/kg bodyweight to about 0.2 mg Ca²⁺ ion/kg bodyweight. Administration of calcium ion doses in an amount of 0.005 mg Ca²⁺ ion/kg bodyweight to about 0.2 mg Ca²⁺ ion/kg bodyweight are referred to herein as “mid dose” calcium ion administration.

In one embodiment, the calcium formulations may be administered in an amount effective to deliver to the lung calcium ion in an amount of 0.005 mg Ca²⁺ ion/kg bodyweight to about 0.5 mg Ca²⁺ ion/kg bodyweight.

If desired, the calcium formulations may be administered in an amount effective to deliver to the lung calcium ion in an amount of less than 0.02 mg Ca²⁺ ion/kg bodyweight, less than 0.01 mg Ca²⁺ ion/kg bodyweight, or less than 0.005 mg Ca²⁺ ion/kg bodyweight. Administration of calcium ion doses in an amount of less than 0.02 mg Ca²⁺ ion/kg bodyweight, less than 0.01 mg Ca²⁺ ion/kg bodyweight, or less than 0.005 mg Ca²⁺ ion/kg bodyweight are referred to herein as “low dose” calcium ion administration.

Provided herein are methods for treating respiratory diseases (e.g. chronic airway diseases and pulmonary diseases) and respiratory conditions (including acute conditions, such as acute pathogenic infections, inflammations and irritations). Further provided herein are methods for the prevention and/or treatment (e.g. attenuation of severity) of acute exacerbations of respiratory diseases (e.g. chronic airway diseases and pulmonary diseases). Respiratory diseases include, for example, cystic fibrosis (CF) and chronic obstructive pulmonary disease (COPD). These diseases are usually associated with chronic airway inflammation and excess airway mucus that predispose to and result in acute exacerbations that can be triggered by acute infections (e.g. viral or bacterial infections) or other environmental inhaled respiratory insults. Respiratory diseases also include, for example, asthma and other respiratory diseases that are not associated with excess airway mucus.

The methods comprise administering to a subject in need thereof calcium ion in an amount effective to treat (prevent, control, or diminish the severity of) a respiratory disease or a respiratory condition, wherein the amount of calcium ion that is effective may be selected from a low, mid, or high dose range of calcium ions delivered to the lung as described herein, optionally further comprising administering one or more therapeutic agents.

Alternatively, the methods comprise administering to a subject in need thereof calcium ion in an amount effective to prevent and/or treat acute exacerbations of a respiratory disease (e.g. a chronic airway disease or a pulmonary disease), wherein the amount of calcium ion that is effective may be selected from a low, mid, or high dose range of calcium ions delivered to the airways and lung as described herein, optionally further comprising administering one or more additional therapeutic agents.

In some aspects, the invention relates to a method for treating a respiratory disease, e.g., a chronic airway disease or a pulmonary disease, such as asthma, airway hyperresponsiveness, seasonal allergic allergy, bronchiectasis, chronic bronchitis, emphysema, chronic obstructive pulmonary disease, cystic fibrosis and the like, comprising administering to the respiratory tract of a subject in need thereof a calcium salt formulation providing calcium ion in a dose described herein, optionally further comprising administering one or more additional therapeutic agents.

In another aspect, the invention relates to a method for the treatment or prevention of acute exacerbations of a respiratory disease, e.g., a chronic airway disease or a pulmonary disease such as asthma, airway hyperresponsiveness, seasonal allergic allergy, bronchiectasis, chronic bronchitis, emphysema, chronic obstructive pulmonary disease, cystic fibrosis and the like, comprising administering to the respiratory tract of a subject in need thereof a calcium salt formulation providing calcium ion in a dose described herein, optionally further comprising administering one or more additional therapeutic agents.

In another aspect, the invention relates to a method for treating and/or reducing the severity of a respiratory disease or respiratory condition, e.g., pulmonary parenchyal inflammatory/fibrotic conditions, such as idiopathic pulmonary fibrosis (IPF), pulmonary interstitial inflammatory conditions (e.g., sarcoidosis, allergic interstitial pneumonitis (e.g., Farmer's Lung)), fibrogenic dust interstitial diseases (e.g., asbestosis, silicosis, beryliosis), eosinophilic granulomatosis/histiocytosis X, collagen vascular diseases (e.g., rheumatoid arthritis, scleroderma, lupus), Wegner's granulomatosis, and the like, comprising administering to the respiratory tract of a subject in need thereof a calcium salt formulation providing calcium ion in a dose described herein, optionally further comprising administering one or more additional therapeutic agents.

A reduction in the severity of an infection may be determined by any suitable method known in the art, including using microbiological assays, e.g. assays suitable to detect a reduction in bacterial colony forming units or a reduction in viral titers. Such assays are described, for example in PCT Publication Nos. WO 2012/030664 “DRY POWDER FORMULATIONS AND METHODS FOR TREATING PULMONARY DISEASES” and WO 2010/111680 “DRY POWDER FORMULATIONS AND METHODS FOR TREATING PULMONARY DISEASES”. A sample, e.g. a sputum sample or blood sample, may be obtained from the subject before treatment to establish a baseline (first, baseline sample) and at one or more points after initiation of the treatment (second sample). A reduction in the severity of an infection may be indicated if the reduction in bacterial colony forming units or the reduction in viral titers is about 0.1 log 10, about 0.2 log 10, about 0.3 log 10, about 0.4 log 10, about 0.5 log 10, about 0.6 log 10, about 0.7 log 10, about 0.8 log 10, about 0.9 log 10, about 1 log 10, about 2 log 10, about 3 log 10, about 4 log 10, about 5 log 10, or about 6 log 10 when the second sample is compared to the baseline sample. A reduction in the severity of an infection may alternatively or additionally be determined using one or more clinical markers, or by assessment of clinical symptoms or signs known to be associated with the infection. For example, infections that are characterized by fever could be assessed by a reduction in the magnitude or duration of fever. A body temperature measurement may be obtained from the subject before treatment to establish a baseline (first, baseline sample) and at one or more points after initiation of the treatment (second sample). A reduction in the severity of an infection may be indicated if the reduction is about 0.3° C., about 0.4° C., about 0.5° C., about 0.6° C., about 0.7° C., about 0.8° C., about 0.9° C., about 1° C., about 2° C., about 3° C., about 4° C., about 5° C., about 6° C., or about 7° C. when the second sample is compared to the baseline sample.

In another aspect, the invention relates to a method for treating, preventing and/or reducing contagion or transmission or reducing the severity of a respiratory disease or respiratory condition associated with a pathogenic infection (e.g. viral or bacterial) of the respiratory tract, comprising administering to the respiratory tract of a subject in need thereof a calcium salt formulation providing calcium ion in a dose described herein, optionally further comprising administering one or more therapeutic agents.

In still another aspect, the invention relates to a method for reducing inflammation of the respiratory tract associated with a respiratory disease (e.g. a chronic airway disease or a pulmonary disease) comprising administering to the respiratory tract of a subject in need thereof a calcium salt formulation providing calcium ion in a dose described herein, optionally further comprising administering one or more therapeutic agents.

A reduction in inflammation may be determined by any suitable method known in the art, including using assays to detect protein or nucleic acid biomarkers that are associated with inflammation (e.g. IL-8, IL-6, GM-CSF, and IL1-beta) or assays that determine the number of inflammatory cells in a suitable sample (e.g. neutrophils or eosinophils), such as those described herein (see, e.g., Examples 4, 5, 9 and 10). A sample, e.g. a sputum sample or blood sample, may be obtained from the subject before treatment to establish a baseline (first, baseline sample) and at one or more points after initiation of the treatment (second sample). A reduction in inflammation may be indicated if the concentration of a protein biomarker in a suitable sample is reduced by, e.g. 0.1 log 10, about 0.2 log 10, about 0.3 log 10, about 0.4 log 10, about 0.5 log 10, about 0.6 log 10, about 0.7 log 10, about 0.8 log 10, about 0.9 log 10, about 1 log 10, about 2 log 10, about 3 log 10, about 4 log 10, about 5 log 10, or about 6 log 10 when the second sample is compared to the baseline sample. A reduction in inflammation may be indicated if the expression of a gene biomarker in a suitable sample is reduced by, e.g. a factor of about 1.5, about 2, about 2.5, about 3, about 3.5, about 4, about 5, or about 10 when the second sample is compared to the baseline sample. A reduction in inflammation may be indicated if the number of inflammatory cells in the sample is reduced by about 0.1 log 10, about 0.2 log 10, about 0.3 log 10, about 0.4 log 10, about 0.5 log 10, about 0.6 log 10, about 0.7 log 10, about 0.8 log 10, about 0.9 log 10, about 1 log 10, about 2 log 10, or about 3 log 10 when the second sample is compared to the baseline sample.

In still another aspect, the invention relates to a method of decreasing an inflammatory response to a soluble or particulate allergen, the method comprising administering to the respiratory tract of a subject in need thereof, preferably an asthma-, CF-, or COPD-indicated subject, a calcium salt formulation providing calcium ion in a dose described herein, optionally further comprising administering one or more therapeutic agents.

In yet another aspect, the invention relates to a method of preventing an inflammatory response to a soluble or particulate allergen, the method comprising administering to the respiratory tract of a subject, preferably an asthma-, CF-, or COPD-indicated subject, a calcium salt formulation providing calcium ion in a dose described herein, optionally further comprising administering one or more therapeutic agents, prior to an encounter with an allergen. Thus, the administration of calcium salt formulations may be prophylactic.

The calcium salt formulations can be used to broadly prevent or treat acute and/or chronic inflammation and, in particular, inflammation that characterizes a number of respiratory diseases and respiratory conditions including, asthma, airway hyperresponsiveness, seasonal allergic allergy, bronchiectasis, chronic bronchitis, emphysema, chronic obstructive pulmonary disease (COPD), cystic fibrosis (CF), pulmonary parenchyal inflammatory diseases/conditions and the like. The calcium salt formulations can be administered to prevent or treat both the inflammation inherent in respiratory diseases like asthma, COPD and CF and the increased inflammation caused by acute exacerbations of those diseases, both of which play a primary role in the pathogenesis of these respiratory diseases.

In still another aspect, the invention relates to a method for reducing acute inflammation, e.g. associated with an irritation and/or an infection in a subject not afflicted with or suffering from a respiratory disease (e.g. a chronic airway disease or a pulmonary disease), the methods comprising administering to the respiratory tract of the subject a calcium salt formulation providing calcium ion in a dose described herein, optionally further comprising administering one or more therapeutic agents.

Regimens comprising the use of calcium formulations suitable for treatment of respiratory diseases (e.g. a chronic airway diseases and a pulmonary diseases) associated with excess airway mucus, chronic inflammation and/or acute exacerbations, e.g., triggered by acute pathogenic infections or environmental insults, include high calcium ion dose regimens. Calcium formulations may be administered in an amount effective to deliver to the lung a high dose of calcium ion, e.g. in an amount of 0.075 mg Ca²⁺ ion/kg bodyweight to about 1.25 mg Ca²⁺ ion/kg bodyweight, preferably in an amount of 0.075 mg Ca²⁺ ion/kg bodyweight to about 0.75 mg Ca²⁺ ion/kg bodyweight, in an amount of 0.1 mg Ca²⁺ ion/kg bodyweight to about 1.0 mg Ca²⁺ ion/kg bodyweight, or in an amount of 0.125 mg Ca²⁺ ion/kg bodyweight to about 1.25 mg Ca²⁺ ion/kg bodyweight. In one embodiment, the regimens comprise the use of calcium formulations that may be administered in an amount effective to deliver to the lung calcium ion in an amount of 0.075 mg Ca²⁺ ion/kg bodyweight to about 0.5 mg Ca²⁺ ion/kg bodyweight.

For example, a calcium salt formulation, if delivered to the lung in an amount of 0.075 mg Ca²⁺ ion/kg bodyweight to about 1.25 mg Ca²⁺ ion/kg bodyweight may have anti-inflammatory and/or anti-infectious activity, and may augment (promote) mucociliary clearance (MCC). Such treatment is particularly suitable for cystic fibrosis (CF).

Mucociliary clearance (MCC) can be measured, e.g. in animal models such as in sheep or dogs, as described in PCT Publication Nos. WO 2012/030664 “DRY POWDER FORMULATIONS AND METHODS FOR TREATING PULMONARY DISEASES” and WO 2010/111680 “DRY POWDER FORMULATIONS AND METHODS FOR TREATING PULMONARY DISEASES” or by any other suitable test known in the art. Mucociliary clearance can be measured by a well-established technique that measures the function and speed of clearance quantitatively using safe, inhaled radioisotope preparation (e.g., Technitium (^{99m}Tc)) in solution. The radioisotope is measured quantitatively by external scintigraphy. Serial measurements over minutes to several hours allow for the assessment of velocity of clearance and effect of a drug vs. baseline/control value. Hypertonic saline (HS) is an agent typically used to promote MCC. The formulation comprising one or more calcium salts delivered to the lungs at a dose that approximates the same osmotic load and local tonicity as HS may augment MCC to the same extent as HS. The formulations comprising one or more calcium salts may also augment MCC to a larger extent than HS when delivered to the lungs at a dose that approximates the same osmotic load and local tonicity as HS. The effect can, for example, comprise an extended duration of augmentation of MCC compared to HS.

The calcium salt formulations described herein can be administered to increase the rate of mucociliary clearance. Clearance of microbes and inhaled particles is an important function of the airways to prevent respiratory infection and exposure to airway inflammation or other deleterious airway effects, or systemic absorption of potentially noxious agents. Clearance is performed as an integrated function by epithelial, mucus-secreting, and immunologic response cells present at the airway surface. It includes the cilia at the epithelial cell airway surface, whose function is to beat synchronously to transport the overlying liquid mucus blanket proximally (toward the mouth), where it exits the airway and is swallowed or expectorated. Calcium salt formulations when administered in suitable doses described herein may assist in one or more of these functions.

For example, by increasing surface viscoelasticity, the calcium salt formulations retain microbes and particulates at the surface of the airway mucus blanket, where they do not gain access to the epithelial cells lining the airway and/or systemic exposure to the host. Calcium salt formulations may also induce osmotic water/liquid transport out of the airway epithelial cells, hydrating the peri-ciliary layer and thus making it less viscous and rendering ciliary beating more effective in moving and clearing the overlying mucus blanket. Calcium salt formulations may further increase both ciliary beat frequency and the force or vigor of ciliary contractions, with resultant increase in clearance velocity of the overlying mucus stream.

An augmentation of airway mucociliary clearance may be determined by any suitable method known in the art, including using animal models, such as e.g. sheep and dog models, as well as in human subjects as described in Example 3. For scintigraphy, a baseline (e.g. rate of mucus clearance velocity) for the subject may be established using the clearance of radioactivity (using an inhaled radioisotope) after either a vehicle control or preferably untreated. An augmentation of airway mucociliary clearance may be identified when the clearance velocity (e.g. measured as percentage reduction of radioactivity per unit of time) or total cumulative clearance (e.g. measured as total remaining radioactivity over time) for the whole lung or preferably for the central lung region for the treatment measurement is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% increased when compared to the baseline (vehicle control). An augmentation of airway mucociliary clearance may also be determined by demonstrating improvement in spirometry (FEV₁, FVC) or improvement in lung clearance index (LCI) over an extended duration. Improvements in these measures of pulmonary function as a result of augmentation of mucociliary clearance velocity can be identified in days to weeks after the initiation of an agent or therapy effective in augmenting clearance (e.g. over 14 days or 28 days). For example, a mean FEV₁ improvement (increase) of about 30 ml, about 40 ml, about 50 ml, about 60 ml, about 70 ml, about 80 ml, about 90 ml, about 100 ml, about 150 ml, about 200 ml, about 250 ml, about 300 ml, about 400 ml, about 500 ml, about 600 ml, about 700 ml, about 800 ml, about 900 ml or about 1000 ml is indicative of an augmentation of airway mucociliary clearance. A mean LCI improvement (increase) of about 0.5 units, about 1 unit, about 1.5 units, about 2 units, about 2.5 units, about 3 units, about 4 units, about 5 units, about 6 units, about 7 units, about 8 units about 9 units or about 10 units is indicative of an augmentation of airway mucociliary clearance.

A mid calcium ion dose administration regimen may be suitable if less MCC augmentation is desired than that which can be achieved with a high calcium ion dose administration regimen while wishing to maintain the anti-inflammatory and/or anti-infectious effects of the calcium ion dose or if no MCC augmentation is desired. For example, suitable calcium formulations may be administered in an amount effective to deliver to the lung calcium ion in an amount of 0.005 mg Ca²⁺ ion/kg bodyweight to about 0.2 mg Ca²⁺ ion/kg bodyweight, preferably in an amount of 0.005 mg Ca²⁺ ion/kg bodyweight to about 0.05 mg Ca²⁺ ion/kg bodyweight, in an amount of 0.01 mg Ca²⁺ ion/kg bodyweight to about 0.1 mg Ca²⁺ ion/kg bodyweight, or in an amount of 0.02 mg Ca²⁺ ion/kg bodyweight to about 0.2 mg Ca²⁺ ion/kg bodyweight. Such doses may be optimal for managing a disease such as COPD. Many patients affected with COPD show chronic inflammation and are at risk of acute exacerbations, but some may not be as significantly affected by excess airway mucus as experienced by many CF patients. Calcium ion doses of more than about 0.075 mg Ca²⁺ ion/kg bodyweight show augmentation of MCC.

One disadvantage of most commercially available MCC promoting agents is their short duration of effect. A treatment comprising the use of one or more MCC promoting agents may be combined with a calcium ion treatment regimen, e.g. a low dose, mid dose, or high dose calcium ion treatment regimen. Co-administration of calcium ions, e.g. as a liquid or dry powder calcium salt formulation, may prolong the short-lived duration of the MCC promoting effect that is provided by the MCC promoting agent when administered alone. Further, currently available MCC promoting agents when administered alone do not provide anti-inflammatory activity or anti-infectious activity. Thus, a treatment comprising the use of one or more MCC promoting agents may be combined with a high- or mid dose calcium ion treatment regimen to provide anti-inflammatory and or anti-infectious activity that is not provided by the MCC promoting agents. Preferred indications for these types of regimens are CF, COPD, and bronchiectasis.

MCC promoting agents are known in the art and include mannitol, HS, epithelial sodium channel (ENaC) blockers (e.g. Amiloride, benzamil, phenamil, amiloride analogs, as described in Hirsh J A, et al. J Pharm Exp Ther, 311:929-37 (2004), N-(3,5-Diamino-6-chloropyrazine-2-carbonyl)-N′-4-[4-(2,3-dihydroxypropoxy)phenyl]butyl-guanidine Methanesulfonate (552-02), as described in Hirsh J A, et al. J Pharm Exp Ther, 325:77-88 (2008)), channel-activating protease inhibitors (CAP inhibitors, e.g. Camostat, as described in Coote K. et al. J Pharm Exp Ther, 329:764-74 (2009)), P2Y₂-receptor agonists (e.g. INS365, as described in Sabater J R et al. J Appl Physiol 87:2191-96 (1999)), ATP, UTP, SABA (Albuterol), LABA (Salmeterol), and leucine. The calcium salt formulation may be administered before administration of the MCC promoting agent, concurrent therewith, or after administration of the MCC promoting agent.

If desired, e.g. for CF, COPD or bronchiectasis, a high-, mid-, or low calcium ion dose regimen may be combined with co-administration of one or more additional therapeutic agents, such as other mucoactive or mucolytic agents, surfactants, antibiotics, antivirals, antihistamines, cough suppressants, bronchodilators, anti-inflammatory agents, steroids, vaccines, adjuvants, expectorants, antifibrotic agents, macromolecules, etc.

A mid calcium ion dose administration regimen that does not augment MCC or may only marginally augment MCC may be suitable for diseases not generally associated with excess airway mucus, for example, asthma. Calcium ion doses of less than about 0.075 mg Ca²⁺ ion/kg bodyweight show little or no augmentation of MCC, while calcium ion doses of more than about 0.005 mg Ca²⁺ ion/kg bodyweight delivered to the lung have anti-infectious and/or anti-inflammatory effects. A calcium ion dose range of about 0.005 mg Ca²⁺ ion/kg bodyweight to about 0.075 mg Ca²⁺ ion/kg bodyweight or a calcium ion dose range of about 0.005 mg Ca²⁺ ion/kg bodyweight to about 0.1 mg Ca²⁺ ion/kg bodyweight may be preferred for treatment of a chronic inflammatory condition such as asthma and for the prevention of acute exacerbations caused by pathogenic infections or environmental insults.

If desired, a low calcium ion dose regimen may be administered, e.g., calcium ion doses in an amount of less than 0.02 mg Ca²⁺ ion/kg bodyweight, less than 0.01 mg Ca²⁺ ion/kg bodyweight, or less than 0.005 mg Ca²⁺ ion/kg bodyweight, that is combined with co-administration of one or more suitable additional therapeutic agents. Such regimens may be suitable to treat a respiratory disease (e.g. a chronic airway disease or a pulmonary disease) or a respiratory condition (including acute conditions, such as, e.g., acute pathogenic infections, inflammations and irritations). Such regimens may also be suitable to prevent and/or treat acute exacerbations of respiratory diseases (e.g. a chronic airway diseases and a pulmonary diseases). The one or more additional therapeutic agents that may be combined with a low dose calcium ion regimen can be, for example, agents that augment MCC, agents that are anti-inflammatory and/or anti-infectious agents. The calcium salt formulation may be administered before administration of the one or more additional therapeutic agents, concurrent therewith, or after administration of the one or more additional therapeutic agents. These can include any known, effective, approved and available agents for the treatment of respiratory diseases, e.g. mucoactive or mucolytic agents, surfactants, antibiotics, antivirals, antihistamines, cough suppressants, bronchodilators, anti-inflammatory agents, steroids, vaccines, adjuvants, expectorants, antifibrotic agents, macromolecules, etc.

A low calcium ion dose regimen when administered alone (i.e. in the absence of co-administration of additional therapeutic agents) may or may not have a measurable pharmacological effect, e.g. a biological activity selected from anti-bacterial activity, anti-viral activity, anti-inflammatory activity, MCC augmenting activity and combinations thereof. A pharmacological effect can be easily evaluated using in vivo models known in the art and described herein.

For example, anti-infectious activity includes anti-bacterial activity and anti-viral activity, which can be determined by a reduction in colony forming units recovered from the lung in the mouse model of bacterial pneumonia or a reduction in nasal wash viral titer in a ferret model of influenza infection as described in PCT Publication No. WO 2012/030664 “DRY POWDER FORMULATIONS AND METHODS FOR TREATING PULMONARY DISEASES” or as determined by any other suitable test known in the art.

For example, anti-inflammatory activity can be determined by measuring the degree of reduction in inflammatory cells including total leukocytes and/or neutrophils recovered from the airway or lung in the tobacco smoke mouse model of COPD as described in PCT Publication No. WO 2012/030664 “DRY POWDER FORMULATIONS AND METHODS FOR TREATING PULMONARY DISEASES” or as determined by any other suitable test known in the art.

The low calcium ion dose (e.g., calcium ion doses in an amount of less than 0.02 mg Ca²⁺ ion/kg bodyweight, less than 0.01 mg Ca²⁺ ion/kg bodyweight, or less than 0.005 mg Ca²⁺ ion/kg bodyweight) may be sufficient to alter the airway mucosal lining fluid (e.g. sufficient to alter the surface tension, surface viscosity, surface elasticity, and/or viscoelasticity of the mucosal lining) to potentiate uptake of a therapeutic agent, e.g. mucoactive or mucolytic agents, surfactants, antibiotics, antivirals, antihistamines, cough suppressants, bronchodilators, anti-inflammatory agents, steroids, vaccines, adjuvants, expectorants, antifibrotic agents, and macromolecules.

For example, the dose administered to a mucosal surface may make the mucosal lining more liquid-like, as described, for example, in U.S. Publication No. 2007/0053844. It is postulated that calcium salt formulations may also act as osmotic agents that lead to mucus hydration, which may lead to enhanced uptake of therapeutic agents into the airway epithelium. Preferred doses of calcium ions delivered to the respiratory tract capable of modulating the mucosal lining fluid are calcium ion doses in an amount of less than 0.02 mg Ca²⁺ ion/kg bodyweight, less than 0.01 mg Ca²⁺ ion/kg bodyweight, and less than 0.005 mg Ca²⁺ ion/kg bodyweight. These calcium ion doses are preferred to promote or augment the activity of therapeutic agents and/or to enable lowering their respective effective doses in disease management.

If desired, high-, mid- or low dose calcium ion administration regimens may not include additional therapeutic agents or may include administration of one or more therapeutic agents. For example, calcium ion administration regimens comprising the use of dry powder calcium salt formulations may be administered together with a bronchodilator. The bronchodilator may, for example, be administered before or after the calcium salt formulation. The bronchodilator may be administered 1 minute, 5 minutes, 10 minutes, 20 minutes, 30 minutes, 45 minutes, 1 hour, 2 hours or 3 hours before administration of the calcium salt formulation. Alternatively, the bronchodilator may be administered if needed after administration of the calcium salt formulation, e.g. if a bronchoconstriction event occurs.

When the patient is pretreated with a bronchodilator it is preferred that the dry powder calcium salt formulation is administered at a time after the bronchodilator when the onset of bronchodilatory effect is evident or, more preferably, maximal. For example, a short-acting beta₂-agonist (SABA) such as albuterol can be administered about 10 minutes to about 30 minutes, preferably, about 15 minutes, prior to administration of the dry powder calcium salt formulation. Pretreatment with a short-acting beta₂-agonist such as albuterol is particularly preferred for CF patients. Some patients may already be taking bronchodilators, such as LABAs (long-acting beta₂-agonist, e.g., formoterol) or LAMAs (long-acting long-acting inhaled muscarinic antagonist, e.g. tiotropium). Patients with COPD frequently take long-acting inhaled bronchodilators to manage their disease. Patients that are taking LABAs or LAMAs already receive some degree of bronchodilation due to the effects of the inhaled bronchodilator agent, and therefore further bronchodilation (e.g., using a short-acting beta₂-agonist) may not be required or desired. For these types of patients, dry powder calcium salt formulations can be administered at substantially the same time or concurrently with the LABA or LAMA, for example, in a single formulation.

Suitable methods for predicting the lung doses of Ca²⁺ ion or any other inhalation therapeutic agent include using the fine particle dose (FPD) measured by cascade impaction techniques such as those described in USP30<601>. Preferably, the fine particle dose of less than 4.4 microns in diameter [FPD(<4.4)] can be measured by an 8-stage Andersen cascade impactor at 60 liter/minute for powder formulations delivered from an RSO1 high resistance (HR) dry powder inhaler (Plastiape, Italy) at a flow rate of 60 liter/minute for 2 seconds. The suitability of using FPD(<4.4) for characterizing lung dose was verified for a calcium dry powder formulation containing 20% (w/w) leucine, 75% (w/w) calcium lactate, 5% (w/w) sodium chloride. The FPD(<4.4) of the dry powder was compared to the lung deposition for the same aerosol size distribution predicted by an empirical lung deposition model (Finlay and Martin, J. Aerosol Med, Vol. 21:189-205, 2008; www.mece.ualberta.ca/arla/aerosoldepositioncalculator_adult.html). For the aerosol distribution of the calcium dry powder formulation at a label claim dose of 10.6 mg Ca²⁺ ion with a mass median aerodynamic diameter (MMAD) of 3.9 microns and geometric standard deviation (GSD) of 1.8, inhaling from the RSO1 HR DPI, the deposition model predicted 4.4 mg Ca²⁺ ion delivered to the lung. The measured FPD(<4.4) for the dry powder was 4.1 mg Ca²⁺, which is equivalent to 0.082 mg Ca²⁺ ion/kg bodyweight for a 50 kg bodyweight person, as typically assumed for such calculations.

For calcium formulations administered by nebulization, about one half of the dose is believed to be exhausted during exhalation and never to actually cross the patient's lips. Therefore, a suitable method includes characterizing the dosing in-vitro with tidal breathing simulation and measuring the dose (fine particle dose (FPD<5.0 micrometers)) delivered to filters.

It should be appreciated by one skilled in the art that the efficacious lung dose can be achieved by delivery of different emitted and labeled doses due to the varying efficiencies of delivery to the lung of different dry powder inhalers and different powder properties. Properties of the DPI such as air flow resistance and properties of the powder such as cohesiveness, affect the resulting air flow rate and particle size distribution which determine the lung dose delivered. It should be appreciated that the dose delivered to the respiratory tract, including oral and/or nasal cavities and upper airways and the lungs is represented by the emitted dose (ED) which can be measured by the amount of therapeutic agent exiting the dry powder inhaler, for example using methods described in USP30<601>. USP30<601> may also be used for dose calculations of calcium ions exiting a nebulized device. In addition, the labeled dose which is metered into a capsule or blister dosage form or metered by the device for delivery to the respiratory tract will again be higher than the emitted dose due to the losses of drug on interior surfaces of the DPI and dosage unit such as capsule or blister. For example, with a calcium dry powder formulation (20% (w/w) leucine, 75% (w/w) calcium lactate, 5% (w/w) sodium chloride) delivered from the RS01 HR DPI at 60 LPM, the FPD(<4.4) of 4.1 mg Ca²⁺ ion requires an emitted dose of 8.7 mg Ca²⁺ ion exiting the DPI and a labeled dose of 10.6 mg Ca²⁺ ion filled into a size 3 capsule to achieve a lung dose of 0.082 mg Ca²⁺ ion/kg body weight for a 50 kg person. For example, i) a nominal human dose of 2.8 mg calcium ion (Ca²⁺) corresponds to a nominal powder load of 20 mg of Formulation II, which corresponds to a predicted human lung dose of 1.0 mg calcium ion (Ca²⁺), which corresponds to a predicted human lung dose of 0.020 mg calcium ion (Ca²⁺) per kg bodyweight for a 50 kg person; ii) a nominal human dose of 5.5 mg calcium ion (Ca²⁺) corresponds to a nominal powder load of 40 mg of Formulation II, which corresponds to a predicted human lung dose of 2.1 mg calcium ion (Ca²⁺), which corresponds to a predicted human lung dose of 0.041 mg calcium ion (Ca²⁺) per kg bodyweight for a 50 kg person; iii) a nominal human dose of 11 mg calcium ion (Ca²⁺) corresponds to a nominal powder load of 80 mg of Formulation II, which corresponds to a predicted human lung dose of 4.1 mg calcium ion (Ca²⁺), which corresponds to a predicted human lung dose of 0.082 mg calcium ion (Ca²⁺) per kg bodyweight for a 50 kg person; iv) a nominal human dose of 22 mg calcium ion (Ca²⁺) corresponds to a nominal powder load of 160 mg of Formulation II, which corresponds to a predicted human lung dose of 8.2 mg calcium ion (Ca²⁺), which corresponds to a predicted human lung dose of 0.16 mg calcium ion (Ca²⁺) per kg bodyweight for a 50 kg person. It will be appreciated that the predicted human lung dose per kg bodyweight can be less than the values above because a person may weigh more than 50 kg. For a person weighing 70 kg, for example, the respective predicted human lung dose per kg bodyweight is i) 0.015 for a nominal human dose of 2.8 mg calcium ion (Ca²⁺); ii) 0.030 for a nominal human dose of 5.5 mg calcium ion (Ca²⁺); iii) 0.060 for a nominal human dose of 11 mg calcium ion (Ca²⁺); iv) 0.117 for a nominal human dose of 22 mg calcium ion (Ca²⁺). Thus, in general, the predicted human lung dose per kg bodyweight will range i) from about 0.020 to about 0.010 for a nominal human dose of 2.8 mg calcium ion (Ca²⁺) and a person ranging from 50 kg to 100 kg; ii) from about 0.041 to about 0.021 for a nominal human dose of 5.5 mg calcium ion (Ca²⁺) and a person ranging from 50 kg to 100 kg; iii) from about 0.082 to about 0.041 for a nominal human dose of 11 mg calcium ion (Ca²⁺) and a person ranging from 50 kg to 100 kg; iv) from about 0.16 to about 0.082 for a nominal human dose of 22 mg calcium ion (Ca²⁺) and a person ranging from 50 kg to 100 kg.

It should be appreciated that delivery to the lungs of different doses of calcium ion can be achieved in various ways. For example, the amount of calcium ion can be controlled by the amount of dry powder that is administered to the patient (e.g. via an inhaler), such as the amount of powder in a capsule, blister or reservoir and dosing instructions to the patient (e.g. one, two or more actuations, capsules, etc.). The amount of calcium ion may also be controlled by device design (e.g. controlling the flow rate, amount of de-agglomeration of the powder, etc.). Further, the amount of calcium ion may also be controlled by the powder properties (e.g. dispersibility, particle size, etc.). Additionally, the rate of aerosolization may be controlled for delivery of calcium ions using liquid formulations.

Dry powders comprising calcium salts can be delivered by inhalation at various parts of the breathing cycle (e.g., laminar flow at mid-breath). Breath controlled delivery of nebulized solutions is a recent development in liquid aerosol delivery (Dalby et al. in Inhalation Aerosols, edited by Hickey 2007, p. 437). In this case, nebulized droplets are released only during certain portions of the breathing cycle. For deep lung delivery, droplets are released in the beginning of the inhalation cycle, while for central airway deposition they are released later in the inhalation.

Dry powders comprising calcium salts can be delivered by inhalation to a desired area within the respiratory tract. It is well-known that particles with an aerodynamic diameter of about 1 micron to about 3 microns can be delivered to the deep lung. Larger aerodynamic diameters, for example, from about 3 microns to about 5 microns can be delivered to the central and upper airways.

Dry powders comprising calcium salts suitable for use in the methods described herein can, for example, travel through the upper airways (the oropharynx and larynx), the lower airways, which include the trachea followed by bifurcations into the bronchi and bronchioli, and through the terminal bronchioli which in turn divide into respiratory bronchioli leading then to the ultimate respiratory zone, the alveoli or the deep lung. For example, most of the calcium ions delivered by the calcium salt solutions may deposit in the deep lung, may be delivered primarily to the central airways, or may be delivered primarily to the upper airways.

Aerosol dosage, formulations and delivery systems may be selected for a particular therapeutic application, as described, for example, in Gonda, I. “Aerosols for delivery of therapeutic and diagnostic agents to the respiratory tract,” in Critical Reviews in Therapeutic Drug Carrier Systems, 6: 273-313 (1990); and in Moren, “Aerosol Dosage Forms and Formulations,” in Aerosols in Medicine, Principles, Diagnosis and Therapy, Moren, et al., Eds. Esevier, Amsterdam (1985).

The calcium salt formulations can be administered to the respiratory tract of a subject in need thereof using any suitable method, such as instillation techniques, and/or an inhalation device, such as a dry powder inhaler (DPI) or metered dose inhaler (MDI). For dry powder formulations, some representative capsule-based DPI units are RS-01 (Plastiape, Italy), Turbospin® (PH&T, Italy), Breezhaler® (Novartis, Switzerland), Aerolizer® (Novartis), Podhaler® (Novartis), and Handihaler® (Boehringer Ingelheim (BI), Germany), Inhalators® (BI), Rotahalers® (GlaxoSmithKline (GSK), U.K.), Spinhaler® (Fisons, U.K.), FlowCapss® (Hovione, Portugal) and others known to those skilled in the art. Some representative blister-based DPI units are Diskus® (GSK), Diskhaler® (GSK), Taper Dry) (3M, St. Paul, Minn.), Gemini® (GSK), Twincer® (University of Groningen, Netherlands), Aspirair® (Vectura, U.K.), Acu-Breathe® (Respirics, Raleigh, N.C.), Exubra® (Novartis), Gyrohaler® (Vectura). Omnihaler® (Vectura), Microdose® (Microdose Therapeutx. Monmouth Junction, N.J.) and others known to those skilled in the art. Some representative reservoir-based DPI units are Clickhaler® (Vectura), NEXT DPI (Chiesi, Italy), Easyhaler® (Orion Pharma, U.K.), Novolizer® (Meda Pharam, Germany), Pulmojet® (Sanofi-Aventis, France), Pulvinal® (Chiesi), Skyehaler® (Skyepharma, UK), and Taifun® (Akela Pharma, Austin, Tex.) and others known to those skilled in the art.

Generally, inhalation devices (e.g., DPIs) are able to deliver a maximum amount of dry powder comprising calcium salts in a single inhalation, which is related to the capacity of the blisters, capsules (e.g. size 000, 00, 0E, 0, 1, 2, 3, and 4, with respective volumetric capacities of 1.37 ml, 950 microliter, 770 microliter, 680 microliter, 480 microliter, 360 microliter, 270 microliter, and 200 microliter) or other means that contain the dry powders within the inhaler. Accordingly, delivery of a desired dose or effective amount of calcium ions may require two or more inhalations. Preferably, each dose that is administered to a subject in need thereof contains an effective amount of calcium ions and is administered using no more than about 4 inhalations. For example, each dose of calcium ions can be administered in a single inhalation or 2, 3, or 4 inhalations. For dry powder calcium salt formulations, the desired dose or amount of calcium ions is preferably administered in a single, breath-activated step using a breath-activated DPI. When this type of device is used, the energy of the subject's inhalation both disperses the respirable dry particles and draws them into the respiratory tract.

Suitable intervals between doses that provide the desired therapeutic effect can be determined based on the severity of the condition (e.g., infection, irritation, or inflammation), overall well being of the subject and the subject's tolerance to calcium salt formulations (e.g. delivered as respirable dry powders or in liquid aerosolized form) and other considerations. Based on these and other considerations, a clinician can determine appropriate intervals between doses. Generally, calcium salt formulations may be administered once, twice or three times a day, as needed.

Alternatively, or in addition, the amount of calcium ion can be controlled by the formulation of the dry powder and dry particles or liquid. For example, the amount of calcium provided can vary depending upon the particular salt selected and dosing can be based on the desired amount of calcium to be delivered to the lung. For example, one mole of calcium chloride (CaCl₂) dissociates to provide one mole of Ca²⁺, but one mole of calcium citrate can provide three moles of Ca²⁺.

Preferably, calcium ions are delivered to the lung in the form of dry powders or dry particles or in the form of an aerosolized liquid formulation.

If desired, the calcium formulation can be a dry powder comprising dry particles. Certain preferred dry powders can have one or more preferred characteristics, e.g. the respirable dry particles preferably are small (e.g., VMGD at 1.0 bar of 10 microns or less, preferably 5 microns or less) and dispersible (i.e., possessing 1/4 bar and/or 0.5/4 bar ratios of 2.2 or less, preferably 2.0 or less, or 1.5 or less, as described herein). Preferably, the MMAD of the respirable dry particles is from about 0.5 microns to about 10 microns, more preferably from about 1 micron to about 5 microns. Preferably, the respirable dry particles are also calcium dense, and/or have a tap density of greater than about 0.4 g/cc to about 1.2 g/cc, preferably between about 0.45 g/cc to about 1.1 g/cc, or 0.55 g/cc and about 1.0 g/cc (gram per cubic centimeter).

Formulations comprising divalent metal cation salts, particularly calcium salts that may be suitable for the methods described herein can be found, for example, in PCT Publication Nos. WO 2006/125153 “FORMULATIONS FOR ALTERATION OF BIOPHYSICAL PROPERTIES OF MUCOSAL LINING”; WO 2010/111640 “ANTI-INFLUENZA FORMULATIONS AND METHODS”; WO 2010/111641 “METHODS FOR TREATING AND PREVENTING PNEUMONIA AND VENTILATOR-ASSOCIATED TRACHEOBRONCHITIS”; WO 2010/111644 “PHARMACEUTICAL FORMULATIONS AND METHODS FOR TREATING RESPIRATORY TRACT INFECTIONS”; WO 2010/111650 “CALCIUM CITRATE AND CALCIUM LACTATE FORMULATIONS FOR ALTERATION OF BIOPHYSICAL PROPERTIES OF MUCOSAL LINING”; WO 2010/111680 “DRY POWDER FORMULATIONS AND METHODS FOR TREATING PULMONARY DISEASES”; WO 2012/030645 “RESPIRABLY DRY POWDER COMPRISING CALCIUM LACTATE, SODIUM CHLORIDE AND LEUCINE”; WO 2012/030647 “TREATMENT OF CYSTIC FIBROSIS USING CALCIUM LACTATE, LEUCINE AND SODIUM CHLORIDE IN A RESPIRAPLE DRY POWDER” and WO 2012/030664 “DRY POWDER FORMULATIONS AND METHODS FOR TREATING PULMONARY DISEASES”, the entire content of which is incorporated herein by reference.

The calcium salt formulations usually contain calcium ion in the form of a calcium salt. Suitable calcium salts include, for example, calcium chloride, calcium sulfate, calcium lactate, calcium citrate, calcium carbonate, calcium acetate, calcium phosphate, calcium alginate, calcium stearate, calcium sorbate, calcium gluconate and the like.

Optionally, the calcium salt formulation further comprises any one or more of: i) a monovalent metal cation salt (e.g. sodium salt, potassium salt, and lithium salt), ii) a pharmaceutically acceptable excipient (other than the monovalent cation salt in (i)), and/or iii) a therapeutic agent.

Suitable sodium salts include, for example, sodium chloride, sodium citrate, sodium sulfate, sodium lactate, sodium acetate, sodium bicarbonate, sodium carbonate, sodium stearate, sodium ascorbate, sodium benzoate, sodium biphosphate, sodium phosphate, sodium bisulfite, sodium borate, sodium gluconate, sodium metasilicate and the like.

Suitable lithium salts include, for example, lithium chloride, lithium bromide, lithium carbonate, lithium nitrate, lithium sulfate, lithium acetate, lithium lactate, lithium citrate, lithium aspartate, lithium gluconate, lithium malate, lithium ascorbate, lithium orotate, lithium succinate and any combination thereof.

Suitable potassium salts include, for example, potassium chloride, potassium bromide, potassium iodide, potassium bicarbonate, potassium nitrite, potassium persulfate, potassium sulfite, potassium bisulfite, potassium phosphate, potassium acetate, potassium citrate, potassium glutamate, dipotassium guanylate, potassium gluconate, potassium malate, potassium ascorbate, potassium sorbate, potassium succinate, potassium sodium tartrate and any combination thereof.

If desired, the calcium salt formulations may comprise one or more additional salts, such as one or more non-toxic salts of the elements magnesium, aluminum, silicon, scandium, titanium, vanadium, chromium, cobalt, nickel, copper, manganese, zinc, tin, silver and the like.

If desired, the calcium salt formulations can include a physiologically or pharmaceutically acceptable carrier or excipient. For example, a pharmaceutically-acceptable carrier or excipient includes any of the standard carbohydrate, sugar alcohol, and amino acid carriers that are known in the art to be useful excipients for inhalation therapy, either alone or in any desired combination. Suitable carriers or excipients generally can be relatively free-flowing particulates, may preferably not thicken or polymerize upon contact with water, preferably are toxicologically innocuous when inhaled and preferably do not significantly interact with the therapeutic agent in a manner that adversely affects the desired physiological action of the calcium salts and/or therapeutic agents. Carbohydrate excipients that are useful in this regard include the mono- and polysaccharides. Representative monosaccharides include carbohydrate excipients such as dextrose (anhydrous and the monohydrate; also referred to as glucose and glucose monohydrate), galactose, mannitol, D-mannose, sorbose and the like. Representative disaccharides include lactose, maltose, sucrose, trehalose and the like. Representative trisaccharides include raffinose and the like. Other carbohydrate excipients include maltodextrin and cyclodextrins, such as 2-hydroxypropyl-beta-cyclodextrin can be used as desired. Representative sugar alcohols include mannitol, sorbitol and the like. A preferred carrier is lactose. Optionally, magnesium stearate or leucine may be blended with the carrier. The carriers can be blended with the calcium salt formulation. The carrier can be between 20 and 80 microns. 80 to 120 microns, 120 to 200 microns. Optionally, smaller carrier particles, such as, for example, between 1 and 20 microns may be blended with the carrier particles.

Suitable amino acid excipients include any of the naturally occurring amino acids that are commonly used with standard pharmaceutical processing techniques and include the non-polar (hydrophobic) amino acids and polar (uncharged, positively charged and negatively charged) amino acids, such amino acids are of pharmaceutical grade and are generally regarded as safe (GRAS) by the U.S. Food and Drug Administration. Representative examples of non-polar amino acids include alanine, isoleucine, leucine, methionine, phenylalanine, proline, tryptophan and valine. Representative examples of polar, uncharged amino acids include cystine, glycine, glutamine, serine, threonine, and tyrosine. Representative examples of polar, positively charged amino acids include arginine, histidine and lysine. Representative examples of negatively charged amino acids include aspartic acid and glutamic acid. These amino acids are generally available from commercial sources that provide pharmaceutical-grade products such as the Aldrich Chemical Company, Inc., Milwaukee, Wis. or Sigma Chemical Company, St. Louis, Mo. Suitable amino acids include glycine, alanine, leucine, and isoleucine. A preferred amino acid is leucine.

Additional excipients include, for example, sugars (e.g., lactose, trehalose, maltodextrin), polysaccharides (e.g. dextrin, maltodextrin, dextran, raffinose), and sugar alcohols (e.g., mannitol, xylitol, sorbitol). In some embodiments, suitable excipients include, for example, dipalmitoylphosphosphatidylcholine (DPPC), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, phospholipids, alkylated sugars, sodium phosphate, maltodextrin, human serum albumin (e.g., recombinant human serum albumin), biodegradable polymers (e.g., PLGA), dextran, dextrin, citric acid, sodium citrate, and the like. In other embodiments, suitable excipients do not include phospholipids. In certain embodiments, dipalmitoylphosphosphatidylcholine (DPPC), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, and phospholipids are specifically excluded as an excipient.

Calcium salt formulations (e.g. liquid formulations or dry powder formulations) particularly suitable for the methods described herein may contain a percentage of calcium ions, e.g. in the form of a calcium salt, of about 0.01% or more, 0.05% or more, 0.1% or more, 0.25% or more, 0.5% or more, 0.75% or more, 1% or more, 1.25% or more, 1.5% or more, 1.75% or more, 2% or more, 2.5% or more, 3% or more, 3.5% or more, 4% or more, 4.5% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% calcium salt (w/w). Calcium salt formulations particularly suitable for the methods described herein may contain a percentage of calcium ions, e.g. in the form of a calcium salt, in the range of from about 0.01% to 99%, from 0.1% to 95%, from 0.5% to 85%, from 1% to 80%, from 3% to 75%, from 5% to 85%, from 10% to 85%, from 15% to 85%, from 20% to 85%, from 30% to 90%, from 40% to 90%, from 50% to 95%, from 60% to 95%, from 70% to 95%, from 80% to 99%, or from 90% to 99% calcium salt (w/w).

For dry powders, the formulation may preferably contain about 20% or more, 25% or more, 30% or more, 35% or more, 40% or more, 50% or more, 60% or more, 70% or more, 75% or more, 80% or more, 85% or more, 90% or more, or 95% calcium salt (w/w), or from about 0.01% to 99%, from 0.1% to 95%, from 3% to 75%, and from 5% to 85% calcium salt (w/w). For liquid formulations, suitable concentration ranges of the calcium salt can vary from about 0.01% to about 20% (w/w), preferably between 0.1% and about 10%.

If desired, the calcium salt formulations can contain low amounts of calcium ions. The formulation may contain less than about 20%, 15%, 10%, 5%, 3%, 2%, or 1% calcium salt (w/w). Low calcium loading in a dry powder may not produce therapeutic efficacy because the quantity of such a dry powder needed to deliver an effective dose of calcium ion cannot reasonably be administered to a subject by inhalation. Accordingly, such powders contain calcium ion in an amount that does not produce therapeutic efficacy.

If desired, the calcium salt formulations can contain any one or more of: i) a monovalent metal cation salt (e.g. sodium salt, potassium salt, and lithium salt), ii) a pharmaceutically acceptable excipient (other than the monovalent cation salt in (i)), and/or iii) a therapeutic agent. Suitable calcium salt formulations may contain 99% (w/w) or less, 98% (w/w) or less, 97% (w/w) or less, 95% (w/w) or less, 90% (w/w) or less, 85% (w/w) or less, 80% (w/w) or less, 75% (w/w) or less, 70% (w/w) or less, 65% (w/w) or less, 60% (w/w) or less, 50% (w/w) or less, 40% (w/w) or less, 30% (w/w) or less, 25% (w/w) or less, 20% (w/w) or less, 15% (w/w) or less, 10% (w/w) or less, 5% (w/w) or less, 4% (w/w) or less, 3% (w/w) or less, 2% (w/w) or less, 1% (w/w) or less of any one or more of the monovalent metal cation salt, the pharmaceutically acceptable excipient, and/or the therapeutic agent. Suitable calcium salt formulations may contain a monovalent metal cation salt, a pharmaceutically acceptable excipient (other than monovalent cation salt), and/or a therapeutic agent in the amount of, for example, from 0.01% to 99%, from 0.1% to 95%, from 0.5% to 85%, from 1% to 80%, from 3% to 75%, from 5% to 85%, from 10% to 85%, from 15% to 85%, from 20% to 85%, from 30% to 90%, from 40% to 90%, from 50% to 95%, from 60% to 95%, from 70% to 95%, from 80% to 99%, or from 90% to 99% (w/w).

Alternatively or in addition, the calcium salt formulation may contain 0.01% or more, 0.1% or more, 0.5% or more, 1% or more, 1.5% or more, 2% or more, 3% or more, 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 99% or more of any one or more of a monovalent metal cation salt, a pharmaceutically acceptable excipient (other than monovalent cation salt), and/or a therapeutic agent.

If desired, the calcium salt formulations can contain one or more therapeutic agents, wherein the one or more therapeutic agent(s) is present in a concentration of about 0.01% (w/w) to about 10% (w/w), or about 0.01% (w/w) to about 20% (w/w), or about 0.01% (w/w) to about 90% (w/w), or about 20% (w/w) to about 90% (w/w), or about 20% (w/w) to about 80% (w/w), or about 20% (w/w) to about 60% (w/w), or about 20% (w/w) to about 50% (w/w), or about 50% (w/w) to about 90% (w/w), or about 50% (w/w) to about 80% (w/w), or about 60% (w/w) to about 90% (w/w), or about 60% (w/w) to about 80% (w/w).

Certain calcium salt formulations when delivered to a subject in the suitable dose ranges described herein, may promote MCC. For example, the formulation may contain a low amount of calcium salt, (e.g. less than about 20%, 15%, 10%, 5%, 3%, 2%, or 1% calcium salt (w/w)), and a high amount of a MCC promoting agent, such as, e.g. mannitol, HS, epithelial sodium channel (ENaC) blockers (e.g. Amiloride, benzamil, phenamil, amiloride analogs), channel-activating protease inhibitors (CAP inhibitors, e.g. Camostat), P2Y₂-receptor agonists (e.g. INS365), ATP, UTP, SABA (Albuterol), LABA (Salmeterol), leucine, or a combination thereof, in an amount of 99% or more (w/w), 98% or more (w/w), 97% or more (w/w), 95% or more (w/w), 90% or more (w/w), 85% or more (w/w), 80% or more (w/w), 70% or more (w/w), 60% or more (w/w), 50% or more (w/w), 40% or more (w/w), 30% or more (w/w), 20% or more (w/w), 10% or more (w/w), optionally further comprising i) a monovalent metal cation salt (e.g. sodium salt, potassium salt, and lithium salt), ii) one or more additional pharmaceutically acceptable excipient(s), and/or iii) one or more additional therapeutic agent(s).

Certain calcium salt formulations when delivered to a subject in the suitable dose ranges described herein, may promote anti-inflammatory, anti-infectious, and/or MCC augmenting activities in a subject. For example, respirable dry powders comprised of dry particles that contain calcium lactate, sodium chloride and leucine are particularly preferred calcium salt formulations. The respirable dry powders may comprise respirable dry particles that contain about 20% (w/w) to about 37.5% (w/w) leucine, about 58.6% (w/w) to about 75% (w/w) calcium lactate, and about 3.9% (w/w) to about 5% (w/w) sodium chloride. An exemplary dry powder contains dry particles that comprise i) about 20% (w/w) leucine, ii) about 75% (w/w) calcium lactate, and iii) about 5% (w/w) sodium chloride. Another exemplary dry powder contains dry particles that comprise i) about 37.5% (w/w) leucine, ii) about 58.6% (w/w) calcium lactate, and iii) about 3.9%/o (w/w) sodium chloride. Other respirable dry powders containing calcium salts are also suitable, e.g. respirable dry powders that comprise respirable dry particles that contain calcium lactate, sodium chloride, one or more additional therapeutic agents and optionally leucine, wherein the dry particles comprise on a dry basis:
A. about 60% to about 75% (w/w) calcium lactate, about 2% to about 5% (w/w) sodium chloride, about 15% to about 20% (w/w) leucine, and up to about 20% (w/w) of one or more additional therapeutic agents;
B. about 45.0% to about 58.6% (w/w) calcium lactate, about 1.9% to about 3.9% (w/w) sodium chloride, about 27.5% to about 37.5% (w/w) leucine, and up to about 20% (w/w) of one or more additional therapeutic agent;
C. about 75% (w/w) calcium lactate, about 5% (w/w) sodium chloride, about 0.01% to about 20% (w/w) of one or more additional therapeutic agents, and about 20% (w/w) or less leucine; or
D. about 58.6% (w/w) calcium lactate, about 3.9% (w/w) sodium chloride, about 0.01% to about 37.5% (w/w) of one or more additional therapeutic agents, and about 37.5% (w/w) or less leucine.

Suitable examples of calcium salt formulations are Formulations I and V, which are liquid formulations and Formulations II, III, and IV, which are dry powder calcium salt formulations. Formulation I is 9.4% CaCl₂ (w/v), 0.62% NaCl (w/v) in water (0.85 M CaCl₂ in 0.11 M NaCl), at a concentration resulting in a tonicity factor of 8 times isotonic. Formulation II contains respirable dry particles that contain 20% (w/w) leucine, 75% (w/w) calcium lactate, and 5% (w/w) sodium chloride. Formulation III contains respirable dry particles that contain 37.5% (w/w) leucine, 58.6% (w/w) calcium lactate, and 3.9% (w/w) sodium chloride. Formulation IV contains respirable dry particles that contain 10% leucine, 58.6% calcium lactate, 31.4% sodium chloride. Formulation V is 1.29% CaCl₂ (w/v), 0.9% NaCl (w/v) in water (0.12 M CaCl₂ in 0.15 M NaCl).

Suitable respirable dry powders that comprise calcium salts and sodium salts may have a ratio of calcium ion to sodium ion (mole:mole) of about 1:1 to about 16:1, about 2:1 to about 16:1, about 4:1 to about 16:1, or about 1:1 to about 8:1, or about 1:1 to about 4:1, or about 1:1 to about 3.9:1, or about 1:1 to about 3.5:1, or about 2:1 to about 8:1, or about 2:1 to about 4:1, or about 2:1 to about 3.9:1, or about 2:1 to about 3:5, about 2:1, about 3:1, about 4:1, about 5:1, about 6:1, about 7:1, or about 8:1; preferably about 4:1.

When it is desirable to retain the relative proportions of calcium salt, monovalent metal cation salt and excipient of any of the particular dry powder formulations described herein, a therapeutic agent can, for example, be added to a solution of the components of the dry powder and the resulting solution spray dried to produce dry particles that contain the therapeutic agent. Spray drying is described, e.g., in PCT Publication Nos. WO 2012/030664 “DRY POWDER FORMULATIONS AND METHODS FOR TREATING PULMONARY DISEASES” and WO 2010/111680 “DRY POWDER FORMULATIONS AND METHODS FOR TREATING PULMONARY DISEASES”. For example, the formulation can contain up to about 1%, about 5%, about 10%, about 20%, about 30%, about 40%, about 50%, about 60%, about 70%, about 80%, or about 90% (w/w) therapeutic agent, and the amount of each of calcium salt, monovalent salt and excipient are reduced proportionally, but the ratio of the amounts (wt %) of calcium salt to monovalent salt to excipient is maintained.

Suitable dry powder calcium salt formulations include blends of respirable dry particles comprising calcium salts and one or more other dry powders or particles, such as dry particles or powders that contain another therapeutic agent or that consist of or consist essentially of one or more pharmaceutically acceptable excipients.

If desired or indicated, the calcium salt formulations described herein can be administered with one or more other active (therapeutic) agents. The therapeutic agents can be administered by any suitable route, such as orally, parenterally (e.g., intravenous, intra-arterial, intramuscular, or subcutaneous injection), topically, by inhalation (e.g., intra-bronchial, intranasal or oral inhalation, intranasal drops), rectally, vaginally, and the like. The calcium salt formulations can be administered before, substantially concurrently with, or subsequent to administration of the therapeutic agent. If the calcium salt formulation is administered in a dose that has anti-inflammatory, anti-infectious and/or MCC promoting activity, it is preferred that the calcium salt formulation and the therapeutic agent are administered so as to provide substantial overlap of their pharmacologic activities. If the calcium salt formulation is administered in a mid- to low calcium ion dose it is preferred that the calcium salt formulation and the therapeutic agent are administered so that the calcium salt formulation may aide the therapeutic agent to provide a pharmacologic activity. For example, the calcium salt formulation may alter the mucosal lining as described herein.

Alternatively or in addition, e.g. when the calcium salt formulation is formulated as a dry powder the therapeutic agent(s) can be blended with the calcium salt formulations described herein, or co-formulated (e.g., spray dried) as desired, e.g., as described in PCT Publication Nos. WO 2012/030664 “DRY POWDER FORMULATIONS AND METHODS FOR TREATING PULMONARY DISEASES” and WO 2010/111680 “DRY POWDER FORMULATIONS AND METHODS FOR TREATING PULMONARY DISEASES”. Alternatively or in addition, the calcium salt formulation may be formulated as a liquid formulation or as a different form of oral formulation that further comprises a suitable therapeutic agent.

Any of the therapeutic agents described herein may be administered in the form of a salt, ester, amide, pro-drug, active metabolite, isomer, analog, fragment, and the like, provided that the salt, ester, amide, pro-drug, active metabolite, isomer, analog or fragment, is pharmaceutically acceptable and pharmacologically active in the present context. Salts, esters, amides, pro-drugs, metabolites, analogs, fragments, and other derivatives of the therapeutic agents may be prepared using standard procedures known to those skilled in the art and described in, for example, J. March, Advanced Organic Chemistry: Reactions, Mechanisms and Structure, 4th Edition (New York: Wiley-Interscience, 1992).

Suitable therapeutic agents that may be used in the methods described herein and that may be co-administered or combined with the calcium formulations described herein and/or that may be incorporated in—or are part of—a desired calcium ion dose regimen described herein, include mucoactive or mucolytic agents, surfactants, antibiotics, antivirals, antihistamines, cough suppressants, bronchodilators, anti-inflammatory agents, steroids, vaccines, adjuvants, expectorants, antifibrotic agents, macromolecules, or therapeutics that are helpful for chronic maintenance of cystic fibrosis (CF), such as MCC promoting agents.

MCC promoting agents include mannitol, HS, epithelial sodium channel (ENaC) blockers (e.g. Amiloride, benzamil, phenamil, amiloride analogs), channel-activating protease inhibitors (CAP inhibitors, like Camostat), P2Y₂-receptor agonists (e.g. INS365), ATP, UTP, SABA (Albuterol), LABA (Salmeterol), and leucine.

Preferred therapeutic agents include, but are not limited to, LABAs (e.g., formoterol, salmeterol), short-acting beta agonists (e.g., albuterol), corticosteroids (e.g., fluticasone), LAMAs (e.g., tiotropium), MABAs (e.g., GSK961081, AZD 2115, and LAS190792), antibiotics (e.g., levofloxacin, tobramycin), antibodies (e.g., therapeutic antibodies), hormones, chemokines, cytokines, growth factors, and combinations thereof. When the calcium salt formulation is intended for treatment of CF, preferred additional therapeutic agents are short-acting beta agonists (e.g., albuterol), antibiotics (e.g., levofloxacin), recombinant human deoxyribonuclease I (e.g., domase alfa, also known as DNase), sodium channel blockers (e.g., amiloride), and combinations thereof.

Suitable therapeutic agents include those that disrupt and/or disperse biofilms. Examples of agents to promote disruption and/or dispersion of biofilms include specific amino acid stereoisomers, e.g., D-leucine, D-methionine, D-tyrosine, D-tryptophan, and the like. (Kolodkin-Gal, I., D. Romero, el al. “D-amino acids trigger biofilm disassembly.” Science 328(5978): 627-629.)

Suitable surfactants include L-alpha-phosphatidylcholine dipalmitoyl (“DPPC”), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, phospholipids, and alkylated sugars.

Examples of suitable mucoactive or mucolytic agents include MUC5AC and MUC5B mucins, DNase, N-acetylcysteine (NAC), cysteine, nacystelyn, dornase alfa, gelsolin, heparin, heparin sulfate, P2Y2 agonists (e.g. UTP, INS365), nedocromil sodium, hypertonic saline, and mannitol.

An antibiotic may be desired for treating a bacterial infection. Suitable antibiotics include a macrolide (e.g., azithromycin, clarithromycin and erythromycin), a tetracycline (e.g., doxycycline, tigecycline), a fluoroquinolone (e.g., gemifloxacin, levofloxacin, ciprofloxacin and mocifloxacin), a cephalosporin (e.g., ceftriaxone, defotaxime, ceftazidime, cefepime), a penicillin (e.g., amoxicillin, amoxicillin with clavulanate, ampicillin, piperacillin, and ticarcillin) optionally with a β-lactamase inhibitor (e.g., sulbactam, tazobactam and clavulanic acid), such as ampicillin-sulbactam, piperacillin-tazobactam and ticarcillin with clavulanate, an aminoglycoside (e.g., amikacin, arbekacin, gentamicin, kanamycin, neomycin, netilmicin, paromomycin, rhodostreptomycin, streptomycin, tobramycin, and apramycin), a penem or carbapenem (e.g. doripenem, ertapenem, imipenem and meropenem), a monobactam (e.g., aztreonam), an oxazolidinone (e.g., linezolid), vancomycin, glycopeptide antibiotics (e.g. telavancin), tuberculosis-mycobacterium antibiotics tobramycin, azithromycin, ciprofloxacin, colistin, and the like.

Suitable agents for treating infections with mycobacteria (e.g., Mycobacterium tuberculosis) include an aminoglycoside (e.g. capreomycin, kanamycin, streptomycin), a fluoroquinolone (e.g. ciprofloxacin, levofloxacin, moxifloxacin), isozianid and isozianid analogs (e.g. ethionamide), aminosalicylate, cycloserine, diarylquinoline, ethambutol, pyrazinamide, protionamide, rifampin, and the like.

Suitable antiviral agents include oseltamivir, zanamavir, amantidine, rimantadine, ribavirin, gancyclovir, valgancyclovir, foscavir, Cytogam® (Cytomegalovirus Immune Globulin), pleconaril, rupintrivir, palivizumab, motavizumab, cytarabine, docosanol, denotivir, cidofovir, and acyclovir. Suitable anti-influenza agents include zanamivir, oseltamivir, amantadine, or rimantadine.

Suitable anti-inflammatory agents and/or agents that modulate inflammatory cytokine/chemokine expression or secretion include, e.g. modulators of the NF-kappaB pathway, modulators of MAP kinases, including modulators of p38 kinase, ERK 42/44, and JNK. A suitable modulating agent can be an antibody or aptamer. Antibodies include polyclonal, monoclonal antibodies, fragments thereof, human or humanized versions, chimeric versions, and the like. The modulating agent can be a nucleic acid. Suitable nucleic acids include antisense molecules, RNAi molecules (e.g. siRNA, shRNA, microRNA), aptamers, ribozymes, triplex forming molecules, and the like.

The modulating agent may be targeted to one of the genes, gene products, polypeptides or proteins (referred to herein as “biomarkers”) selected from the group consisting of Adrb1, Aplnr, Areg, Bdnf, Birc5, Bmp6, Brca1, C8a, Calb1, Ccl2/MCP-1, Ccl4, Ccl5, Ccl6, Cc7/MCP-3, Ccl12, Ccl17, Ccl20/MIP-3a, Ccr1, Ccr6, Ccr9, Ccr11, Ccr12, Clec7a, Cmtm5, Creb1, Csf2/GM-CSF, Cxcl1/KC, Cxcl2/MIP-2, Cxcl5/ENA78, Cxcl9, Cxcl10, Cxcl13, Cxcr1, Cxcr4, Cxcr5, Egr1, FasL, Gem, Gpr81, Gusb, Hif1a, Hspb1, Ifngr2, Igfbp3, Il1a, Il1b, Il-6, Il1r2, Il1rn, IL16, Junb, Kcna5, Lef1, Lep, Lif, Nos2, Per1, Pln, Pmaip1, Pou2af1, Ppbp, Prl2c2, Proc, Ptgs2, Rgs3, Serpina1a, Sod2, Thbs1, Tlr1, Tlr8, Tlr9, TNF, and Xcl1, and may modulate the biomarker by either inhibiting it or activating it, depending on which action is desired e.g. by promoting the biomarker's stability or instability (e.g. on the protein level or nucleic acid level), by promoting or blocking the biomarker's expression or translation, by blocking or activating an active site on the biomarker, by blocking or activating binding sites of the biomarker to protein binding partners (homodimeric, heterodimeric, or multimeric) or nucleic acid binding sites (e.g. promoters, silencers, etc.), by sequestering the biomarker in an active or inactive cellular location. The modulating agent need not physically interact with the biomarker, it may have an indirect effect on the activity of the biomarker, e.g. by up- or downregulating a signaling pathway that leads to the biomarker's activation or deactivation, by interacting with an enzyme that modifies the biomarker (e.g. a kinase, phosphatase, (de-)acetylase, (de-)methylase, (de-)ubiquitinase and the like), by promoting stability or instability of a dimerization or multimerization partner, by sequestering a molecule necessary for the activity of the biomarker (e.g. a substrate, phosphate, etc.), and the like.

Anti-inflammatory agents also include compounds that modulate, preferably inhibit/decrease cell signaling by inflammatory molecules like cytokines (e.g., IL-1, IL-4, IL-5, IL-6, IL-9, IL-13, IL-18 IL-25, IFN-alpha, IFN-beta, and others), CC chemokines CCL-1-CCL28 (some of which are also known as, for example, MCP-1, CCL2, RANTES), CXC chemokines CXCL1-CXCL17 (some of which are also known as, for example, IL-8, MIP-2), growth factors (e.g., GM-CSF, NGF, SCF, TGF-beta, EGF, VEGF and others) and/or their respective receptors.

Some examples of the aforementioned anti-inflammatory antagonists/inhibitors include ABN912 (MCP-1/CCL2, Novartis AG), AMG761 (CCR4, Amgen Inc), Enbrel® (TNF, Amgen Inc, Wyeth), huMAb OX40L GENENTECH (TNF superfamily, Genentech Inc, AstraZeneca PLC), R4930 (TNF superfamily, Roche Holding Ltd), SB683699/Firategrast (VLA4, GlaxoSmithKline PLC), CNT0148 (TNF alpha, Centocor, Inc, Johnson & Johnson, Schering-Plough Corp); Canakinumab (IL-1 beta, Novartis); Israpafant MITSUBISHI (PAF/IL-5, Mitsubishi Tanabe Pharma Corporation); IL-4 and IL-4 receptor antagonists/inhibitors: AMG317 (Amgen Inc), BAY169996 (Bayer AG), AER-003 (Aerovance), APG-201 (Apogenix); IL-5 and IL-5 receptor antagonists/inhibitors: MEDI563 (AstraZeneca PLC, MedImmune. Inc), Bosatria (GlaxoSmithKline PLC), Cinquil® (Ception Therapeutic), TMC120B (Mitsubishi Tanabe Pharma Corporation), Bosatria (GlaxoSmithKline PLC), Reslizumab SCHERING (Schering-Plough Corp); MEDI528 (IL-9, AstraZeneca, MedImmune, Inc); IL-13 and IL-13 receptor antagonists/inhibitors: TNX650 GENENTECH (Genentech), CAT-354 (AstraZeneca PLC, MedImmune), AMG-317 (Takeda Pharmaceutical Company Limited), MK6105 (Merck & Co Inc), IMA-026 (Wyeth), IMA-638 Anrukinzumab (Wyeth), MILR1444A/Lebrikizumab (Genentech), QAX576 (Novartis), CNTO-607 (Centocor), MK-6105 (Merck, CSL), Dual IL-4 and IL-13 inhibitors: AIR645/ISIS369645 (ISIS Altair), DOM-0910 (GlaxoSmithKline, Domantis), Pitrakinra/AER001/Aerovant™ (Aerovance Inc), AMG-317 (Amgen), and the like. CXCR2 antagonists include, for example, Reparixin (Dompe S.P.A.), DF2162 (Dompe, S.P.A.), AZ-(AstraZeneca), SB656933 (GlaxoSmithKline PLC), SB332235 (GlaxoSmithKline PLC), SB468477 (GlaxoSmithKline PLC), and SCH527123 (Shering-Plough Corp).

Other anti-inflammatory agents include omalizumab (anti-IgE immunoglobulin Daiichi Sankyo Company, Limited), Zolair (anti-IgE immunoglobulin, Genentech Inc, Novartis AG, Roche Holding Ltd), Solfa (LTD4 antagonist and phosphodiesterase inhibitor, Takeda Pharmaceutical Company Limited), IL-13 and IL-13 receptor inhibitors (such as AMG-317, MILR1444A, CAT-354, QAX576, IMA-638, Anrukinzumab, IMA-026, MK-6105, DOM-0910, and the like), IL-4 and IL-4 receptor inhibitors (such as Pitrakinra, AER-003, AIR-645, APG-201, DOM-0919, and the like), IL-1 inhibitors such as canakinumab, CRTh2 receptor antagonists such as AZD1981 (CRTh2 receptor antagonist, AstraZeneca), neutrophil elastase inhibitor such as AZD9668 (neutrophil elastase inhibitor, from AstraZeneca), GW856553X Losmapimod (P38 kinase inhibitor, GlaxoSmithKline PLC), Arofylline LAB ALMIRALL (PDE-4 inhibitor, Laboratorios Almirall, S.A.), ABT761 (5-LO inhibitor, Abbott Laboratories), Zyflo® (5-LO inhibitor, Abbott Laboratories), BT061 (anti-CD4 mAb, Boehringer Ingelheim GmbH), Corus (inhaled lidocaine to decrease eosinophils, Gilead Sciences Inc), Prograf® (IL-2-mediated T-cell activation inhibitor, Astellas Pharma), Bimosiamose PFIZER INC (selectin inhibitor, Pfizer Inc), R411 (alpha 4 beta 1/alpha 4 beta 7 integrin antagonist, Roche Holdings Ltd), Tilade® (inflammatory mediator inhibitor, Sanofi-Aventis), Orenica® (T-cell co-stimulation inhibitor, Bristol-Myers Squibb Company). Soliris® (anti-C5, Alexion Pharmaceuticals Inc), Entorken® (Farmacija d.o.o.), Excellair® (Syk kinase siRNA, ZaBeCor Pharmaceuticals, Baxter International Inc), KB003 (anti-GM-CSF mAb, KaloBios Pharmaceuticals), Cromolyn sodiums (inhibit release of mast cell mediators): Cromolyn sodium BOEHRINGER (Boehringer Ingelheim GmbH), Cromolyn sodium TEVA (Teva Pharmaceutical Industries Ltd), Intal (Sanofi-Aventis), BI1744CL (oldaterol (beta 2-adrenoceptor antagonist) and tiotropium, Boehringer Ingelheim GmbH), NF kappaB inhibitors, CXR2 antagonists, HLE inhibitors, HMG-CoA reductase inhibitors, and the like.

Modulators of inflammatory cytokine/chemokine expression or secretion include, for example, 2-[(aminocarbonyl)amino]-5-[4-fluorophenyl]-3-thiophenecarboxamide (TPCA-1); doxycycline; NR58-3.14.3; spiropiperidine; N-(6-chloro-9H-beta-carbolin-8-yl) nicotinamide (PS-1145); N-(6-chloro-7-methoxy-9H-beta-carbolin-8-yl)-2-methyl-nicotinamide (ML 120B); N-acetylcysteine (NAC); antagonist anti-CCR2 (CCR2-05) monoclonal antibody; gamma-tocopherol; 2-cyano-3,12-dioxoolean-1,9-dien-28-oic acid (CDDO); 15-deoxy-delta(12,14)-prostaglandin J(2) (15d-PGJ(2)); GRP blocking agent 77427; GRP blocking antibody 2A11; IKK2 inhibitor (IMD-0354); GSK-3 inhibitor 3-(2,4-dichlorophenyl)-4-(1-methyl-1H-indol-3-yl)-1H-pyrrole-2,5-dione (SB216763); dehydroevodiamine; evodiamine; rutaecarpine; 5 alpha-reductase inhibitor finasteride; cordycepin; Nox2 inhibitors; fluoxetine; chymase inhibitor 2-[4-(5-fluoro-3-methylbenzo[b]thiophen-2-yl)sulfonamido-3-methanesulfonylphenyl]thiazole-4-carboxylic acid (TY-51469); TNF-alpha converting enzyme (TACE) and matrix metalloproteinases (MMPs) dual inhibitors: PKF242-484, PKF241-466; CXCR4 antagonist AMD3100; inhibitor of p44/42 MAPK U0126; IKK-selective inhibitors: PS-1145 [N-(6-chloro-9H-beta-carbolin-8-ly) nicotinamide], ML 120B [N-(6-chloro-7-methoxy-9H-beta-carbolin-8-yl)-2-methyl-nicotinamide]; artemisinin; proteasome inhibitors: pyrrolidine dithiocarbamate [PDTC], MG132, PS-341 (bortezomib); bindarit, thromboxane A(2) synthase inhibitor ozagrel; aminopeptidase N inhibitor actinonin; NF-kappa B inhibitor IKK-NBD; p38 MAP kinase inhibitors: SB 203580, SB 202190; neutrophil elastase inhibitor Sivelestat: quercetin (3,3′,4′,5,7-pentahydroxyflavone); N,N-dimethylsphingosine; phosphodiesterase inhibitor pentoxifylline; PKA inhibitor H-89; anti-CCR2-blocking monoclonal antibody MC21; IkappaB-alpha phosphorylation inhibitor BAY 11-7082; alpha-1-antitrypsin; and synthetic metalloprotease inhibitor (RS 113456).

Other suitable anti-inflammatory agents include leukotriene inhibitors, phosphodiesterase 4 (PDE4) inhibitors, other anti-inflammatory agents, and the like.

Suitable leukotriene inhibitors include montelukast (cystinyl leukotriene inhibitors), masilukast, zafirleukast (leukotriene D4 and E4 receptor inhibitors), pranlukast, zileuton (5-lipoxygenase inhibitors), GSK256066 (GlaxoSmithKline PLC), and the like.

Examples of montelukast (cystinyl leukotriene inhibitor) include Singulair® (Merck & Co Inc), Loratadine, montelukast sodium SCHERING (Schering-Plough Corp), MK0476C (Merck & Co Inc), and the like. Examples of masilukast include MCC847 (AstraZeneca PLC), and the like. Examples of zafirlukast (leukotriene D4 and E4 receptor inhibitor) include Accolate® (AstraZeneca PLC), and the like. Examples of pranlukast include Azlaire (Schering-Plough Corp). Examples of zileuton (5-LO) include Zyflo® (Abbott Laboratories), Zyflo CR® (Abbott Laboratories, SkyePharma PLC), Zileuton ABBOTT LABS (Abbott Laboratories), and the like.

Suitable PDE4 inhibitors include cilomilast, roflumilast, oglemilast, tofimilast, arofylline (Almirall), and the like.

Examples of cilomilast formulations include Ariflo (GlaxoSmithKline PLC), and the like. Examples of roflumilast include Daxas® (Nycomed International Management GmbH, Pfizer Inc), APTA2217 (Mitsubishi Tanabe Pharma Corporation), and the like. Examples of oglemilast include GRC3886 (Forest Laboratories Inc), and the like. Examples oftofimilast include Tofimilast PFIZER INC (Pfizer Inc), and the like.

Suitable steroids include corticosteroids, combinations of corticosteroids and LABAs, combinations of corticosteroids and LAMAs, combinations of corticosteroids, LABAs and LAMAs, and the like.

Suitable corticosteroids include budesonide, fluticasone, flunisolide, triamcinolone, beclomethasone, mometasone, ciclesonide, dexamethasone, and the like.

Examples of budesonide formulations include Captisol-Enabled® Budesonide Solution for Nebulization (AstraZeneca PLC), Pulmicort® (AstraZeneca PLC), Pulmicort® Flexhaler (AstraZeneca Plc), Pulmicort® HFA-MDI (AstraZeneca PLC), Pulmicort Respulesx (AstraZeneca PLC), Inflammide (Boehringer Ingelheim GmbH), Pulmicort® HFA-MDI (SkyePharma PLC), Unit Dose Budesonide ASTRAZENECA (AstraZeneca PLC), Budesonide Modulite (Chiesi Farmaceutici S.p.A), CHF5188 (Chiesi Farmaceutici S.p.A), Budesonide ABBOTT LABS (Abbott Laboratories), Budesonide clickhaler (Vestura Group PLC), Miflonide (Novartis AG), Xavin (Teva Pharmaceutical Industries Ltd.), Budesonide TEVA (Teva Pharmaceutical Industries Ltd.), Symbicort® (AstraZeneca K.K., AstraZeneca PLC), VR632 (Novartis AG, Sandoz International GmbH), and the like.

Examples of fluticasone propionate formulations include Flixotide Evohaler (GlaxoSmithKline PLC), Flixotide Nebules (GlaxoSmithKline Plc), Flovent® (GlaxoSmithKline Plc), Flovent® Diskus (GlaxoSmithKline PLC), Flovent® HFA (GlaxoSmithKline PLC), Flovent® Rotadisk (GlaxoSmithKline PLC), Advair® HFA (GlaxoSmithKline PLC, Theravance Inc), Advair Diskus® (GlaxoSmithKline PLC, Theravance Inc.), VR315 (Novartis AG, Vectura Group PLC, Sandoz International GmbH), and the like. Other formulations of fluticasone include fluticasone as Flusonal (Laboratorios Almirall, S.A.), fluticasone furoate as GW685698 (GlaxoSmithKline PLC, Thervance Inc.), Plusvent (Laboratorios Almirall, S.A.), Flutiform® (Abbott Laboratories, SkyePharma PLC), and the like.

Examples of flunisolide formulations include Aerobid® (Forest Laboratories Inc), Aerospan® (Forest Laboratories Inc), and the like. Examples of triamcinolone include Triamcinolone ABBOTT LABS (Abbott Laboratories), Azmacort® (Abbott Laboratories, Sanofi-Aventis), and the like. Examples of beclomethasone dipropionate include Beclovent (GlaxoSmithKline PLC), QVAR® (Johnson & Johnson, Schering-Plough Corp, Teva Pharmacetucial Industries Ltd), Asmabec clickhaler (Vectura Group PLC), Beclomethasone TEVA (Teva Pharmaceutical Industries Ltd), Vanceril (Schering-Plough Corp), BDP Modulite (Chiesi Farmaceutici S.p.A.), Clenil (Chiesi Farmaceutici S.p.A), Beclomethasone dipropionate TEVA (Teva Pharmaceutical Industries Ltd), and the like. Examples of mometasone include QAB149 Mometasone furoate (Schering-Plough Corp), QMF149 (Novartis AG), Fomoterol fumarate, mometoasone furoate (Schering-Plough Corp), MFF258 (Novartis AG, Merck & Co Inc), Asmanex® Twisthaler (Schering-Plough Corp), and the like. Examples of cirlesonide include Alvesco® (Nycomed International Management GmbH, Sepracor, Sanofi-Aventis, Tejin Pharma Limited), Alvesco® Combo (Nycomed International Management GmbH, Sanofi-Aventis), Alvesco® HFA (Nycomed Intenational Management GmbH, Sepracor Inc), and the like. Examples of dexamethasone include DexPak (Merck), Decadron® (Merck), Adrenocot, CPC-Cort-D, Decaject-10, Solurex and the like. Other corticosteroids include Etiprednol dicloacetate TEVA (Teva Pharmaceutical Industries Ltd), and the like.

Other corticosteroids include TPI 1020 (Topigen Pharmaceuticals), GSK685698 also known as fluticasone furoate (GlaxoSmithKline PLC), and GSK870086 (glucocorticoid agonist; GlaxoSmithKline PLC).

Combinations of corticosteroids and LABAs include salmeterol with fluticasone, formoterol with budesonide, formoterol with fluticasone, formoterol with mometasone, indacaterol with mometasone, vilanterol with fluticasone furoate, formoterol and ciclesonide, and the like.

Examples of salmeterol with fluticasone include Plusvent (Laboratorios Almirall, S.A.), Advair® HFA (GlaxoSmithKline PLC), Advair® Diskus (GlaxoSmithKline PLC, Theravance Inc), VR315 (Novartis AG, Vectura Group PLC, Sandoz International GmbH) and the like. Examples of formoterol with budesonide include Symbicort® (AstraZeneca PLC), VR632 (Novartis AG, Vectura Group PLC), and the like. Examples of vilanterol with fluticasone include GSK642444 with fluticasone and the like. Examples of formoterol with fluticasone include Flutiform® (Abbott Laboratories, SkyePharma PLC), and the like. Examples of formoterol with mometasone include Dulera®/MFF258 (Novartis AG, Merck & Co Inc), and the like. Examples of indacaterol with mometasone include QAB149 Mometasone furoate (Schering-Plough Corp), QMF149 (Novartis AG), and the like. Combinations of corticosteroids with LAMAs include fluticasone with tiotropium, budesonide with tiotropium, mometasone with tiotropium, salmeterol with tiotropium, formoterol with tiotropium, indacaterol with tiotropium, vilanterol with tiotropium, and the like. Examples of vilanterol with fluticasone furoate include Revolair®(GSK642444 and GSK685698; GlaxoSmithKline PLC), and the like. Examples of formoterol and ciclesonide are formoterol and ciclesonide (Forest/Nycomed), and the like. Combinations of corticosteroids with LAMAs and LABAs include, for example, fluticasone with salmeterol and tiotropium.

Other anti-asthma molecules include: ARD111421 (VIP agonist, AstraZeneca PLC), AVE0547 (anti-inflammatory, Sanofi-Aventis), AVE0675 (TLR agonist, Pfizer, Sanofi-Aventis), AVE0950 (Syk inhibitor, Sanofi-Aventis), AVE5883 (NK1/NK2 antagonist, Sanofi-Aventis), AVE8923 (tryptase beta inhibitor, Sanofi-Aventis), CGS21680 (adenosine A2A receptor agonist. Novartis AG), ATL844 (A2B receptor antagonist, Novartis AG), BAY443428 (tryptase inhibitor, Bayer AG), CHF5407 (M3 receptor inhibitor, Chiesi Farmaceutici S.p.A.), CPLA2 Inhibitor WYETH (CPLA2 inhibitor, Wyeth), IMA-638 (IL-13 antagonist, Wyeth), LAS 100977 (LABA, Laboratorios Almirall, S.A.), MABA (M3 and beta₂ receptor antagonist, Chiesi Farmaceutici S.p.A), R1671 (mAb, Roche Holding Ltd), CS003 (Neurokinin receptor antagonist, Daiichi Sankyo Company, Limited), DPC168 (CCR antagonist, Bristol-Myers Squibb), E26 (anti-IgE, Genentech Inc), HAE (Genentech), IgE inhibitor AMGEN (Amgen Inc), AMG853 (CRTH2 and D2 receptor antagonist, Amgen), IPL576092 (LSAID, Sanofi-Aventis), EPI2010 (antisense adenosine 1, Chiesi Farmaceutici S.p.A.), CHF5480 (PDE-4 inhibitor. Chiesi Farmaceutici S.p.A.), KI04204 (corticosteroid, Abbott Laboratories), SVT47060 (Laboratorios Salvat, S.A.), VML530 (leukotriene synthesis inhibitor, Abbott Laboratories). LAS35201 (M3 receptor antagonist, Laboratorios Almirall, S.A.), MCC847 (D4 receptor antagonist, Mitsubishi Tanabe Pharma Corporation), MEM 1414 (PDE-4 inhibitor, Roche), TA270 (5-LO inhibitor, Chugai Pharmaceutical Co Ltd), TAK661 (eosinophil chemotaxis inhibitor, Takeda Pharmaceutical Company Limited), TBC4746 (VLA-4 antagonist, Schering-Plough Corp), VR694 (Vectura Group PLC), PLD177 (steroid, Vectura Group PLC), KI03219 (corticosteroid+LABA, Abbott Laboratories), AMG009 (Amgen Inc), AMG853 (D2 receptor antagonist, Amgen Inc);

AstraZeneca PLC:AZD1744 (CCR3/histamine-1 receptor antagonist, AZD1419 (TLR9 agonist), Mast Cell inhibitor ASTRAZENECA, AZD3778 (CCR antagonist), DSP3025 (TLR7 agonist), AZD1981 (CRTh2 receptor antagonist), AZD5985 (CRTh2 antagonist), AZD8075 (CRTh2 antagonist), AZD1678, AZD2098, AZD2392, AZD3825 AZD8848, AZD9215, ZD2138 (5-LO inhibitor), AZD3199 (LABA); AZD2423 (CCR2b antagonist); AZD5069 (CXCR2 antagonist); AZD5423 (Selective glucocorticoid receptor agonist (SEGRA));

GlaxoSmithKline PLC: GW328267 (adenosine A2 receptor agonist), GW559090 (alpha4 integrin antagonist), GSK679586 (mAb), GSK597901 (adrenergic beta₂-agonist), AM103 (5-LO inhibitor), GSK256006 (PDE4 inhibitor), GSK256066, GW842470 (PDE-4 inhibitor), GSK870086 (glucocorticoid agonist), GSK159802 (LABA), GSK256066 (PDE-4 inhibitor), GSK642444 (vilanterol, LABA, adrenergic beta₂-agonist), GSK685698 (ICS, fluticasone furoate), Revolair® (GSK64244/vilanterol and GSK685698/fluticasone furoate), GSK799943 (corticosteroid), GSK573719 (mAchR antagonist), GSK2245840 (SIRT1 Activator); Mepolizumab (anti-IL-5 mAb); and GSK573719 (LAMA), and GSK573719 (LAMA) and vilanterol (LABA);

Pfizer Inc: PF3526299, PF3893787, PF4191834 (FLAP antagonist), PF610355 (adrenergic beta₂-agonist), CP664511 (alpha 4 beta 1/VCAM-1 interaction inhibitor), CP609643 (inhibitor of alpha 4 beta 1/VCAM-1 interactions), CP690550 (JAK3 inhibitor), SAR21609 (TLR9 agonist), AVE7279 (Th1 switching), TBC4746 (VLA-4 antagonist); R343 (IgE receptor signaling inhibitor), SEP42960 (adenosine A3 antagonist):

Sanofi-Aventis: MLN6095 (CrTH2 inhibitor), SAR137272 (A3 antagonist). SAR21609 (TLR9 agonist), SAR389644 (DP1 receptor antagonist), SAR398171 (CRTH2 antagonist), SSR161421 (adenosine A3 receptor antagonist);

Merck & Co Inc: MK0633, MK0633, MK0591 (5-LO inhibitor), MK886 (leukotriene inhibitor), BIO1211 (VLA-4 antagonist); Novartis AG: QAE397 (long-acting corticosteroid), QAK423, QAN747, QAP642 (CCR3 antagonist), QAX935 (TLR9 agonist), NVA237 (LAMA).

The therapeutic agent can also be selected from the group consisting of transient receptor potential (TRP) channel agonists. In certain embodiments, the TRP agonist is a TRPC, TRPV, TRPM and/or TRPA1 subfamily agonist. In some embodiments, the TRP channel agonist is selected from the group consisting of TRPV2, TRPV3, TRPV4, TRPC6, TRPM6, and/or TRPA1 agonist. Suitable TRP channel agonists may be selected from the group consisting of allyl isothiocyanate (AITC), benyzl isothiocyanate (BITC), phenyl isothiocyanate, isopropyl isothiocyanate, methyl isothiocyanate, diallyl disulfide, acrolein (2-propenal), disulfiram (Antabuse®), farnesyl thiosalicylic acid (FTS), farnesyl thioacetic acid (FTA), chlodantoin (Sporostacin®, topical fungicidal), (15-d-PGJ2), 5,8,11,14 eicosatetraynoic acid (ETYA), dibenzoazepine, mefenamic acid, fluribiprofen, keoprofen, diclofenac, indomethacin, SC alkyne (SCA), pentenal, mustard oil alkyne (MOA), iodoacetamine, iodoacetamide alkyne, (2-aminoethyl) methanethiosulphonate (MTSEA), 4-hydroxy-2-noneal (HNE), 4-hydroxy xexenal (HHE), 2-chlorobenzalmalononitrile, N-chloro tosylamide (chloramine-T), formaldehyde, isoflurane, isovelleral, hydrogen peroxide. URB597, thiosulfinate, Allicin (a specific thiosulfinate), flufenamic acid, niflumic acid, carvacrol, eugenol, menthol, gingerol, icilin, methyl salicylate, arachidonic acid, cinnemaldehyde, super sinnemaldehyde, tetrahydrocannabinol (THC or (delta-9)Δ⁹-THC), cannabidiol (CBD), cannabichromene (CBC), cannabigerol (CBG), THC acid (THC-A), CBD acid (CBD-A), Compound 1 (AMG5445), 4-methyl-N-[2,2,2-trichloro-1-(4-chlorophenylsulfanyl)ethyl]benzamide, N-[2,2,2-trichloro-1-(4-chlorophenylsulfanyl)ethyl]acetamid, AMG9090, AMG5445, 1-oleoyl-2-acetyl-sn-glycerol (OAG), carbachol, diacylglycerol (DAG), 1,2-Didecanoylglycerol, flufenamate/flufenamic acid, niflumate/niflumic acid, hyperforin, 2-aminoethoxydiphenyl borate (2-APB), diphenylborinic anhydride (DPBA), delta-9-tetrahydrocannabinol ((delta-9)Δ⁹-THC or THC), cannabiniol (CBN), 2-APB, O-1821, 11-hydroxy-(delta-9)Δ⁹-tetrahydrocannabinol, nabilone, CP55940, HU-210, HU-211/dexanabinol, HU-331, HU-308, JWH-015, WIN55,212-2,2-Arachidonoylglycerol (2-AG), Arvil, PEA, AM404, O-1918, JWH-133, incensole, incensole acetate, menthol, eugenol, dihydrocarveol, carveol, thymol, vanillin, ethyl vanillin, cinnemaldehyde, 2 aminoethoxydiphenyl borate (2-APB), diphenylamine (DPA), diphenylborinic anhydride (DPBA), camphor, (+)-borneol, (−)-isopinocampheol, (−)-fenchone, (−)-trans-pinocarveol, isoborneol, (+)-camphorquinone, (−)-alpha-thujone, alpha-pinene oxide, 1,8-cineole/eucalyptol, 6-butyl-m-cresol, carvacrol, p-sylenol, kreosol, propofol, p-cymene, (−)-isoppulegol, (−)-carvone, (+)-dihydrocarvone, (−)-menthone, (+)-linalool, geraniol, 1-isopropyl-4-methylbicyclo[3.1.0]hexan-4-ol, 4 alpha PDD, GSK1016790A, 5′6′Epoxyeicosatrienoic (5′6′-EET), 8′9′Epoxyeicosatrienoic (8′9′-EET), APP44-1, RN 1747, Formulation Ib WO 2006/02909, Formulation IIb WO 2006/02909, Formulation IIc WO 2006/02929, Formulation IId WO 2006/02929, Formulation IIIb WO 2006/02929, Formulation IIIc WO 2006/02929, arachidonic acid (AA), 12-O-Tetradecanoylphorbol-13-acetate (TPA)/phorbol 12-myristate 13-acetate (PMA), bisandrographalide (BAA), incensole, incensole acetate, Compound IX WO 2010/015965, Compound X WO 2010/015965, Compound XI WO 2010/015965, Compound XII WO 2010/015965, WO 2009/004071, WO 2006/038070, WO 2008/065666, Formula VII WO 2010/015965, Formula IV WO 2010/015965, dibenzoazepine, dibenzooxazepine, Formula I WO 2009/071631, N-{(1S)-1-[({(4R)-1-[(4-chlorophenyl)sulfonyl]-3-oxohexahydro-1Hazepin-4-yl}amino)carbonyl]-3-methylbutyl}-1-benzothiophen-2-carboxamide, N-{(1S)-1-[({(4R)-1-[(4-fluorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(2-cyanophenyl)sulfonyl]-3-oxohexahydro-1H-azepin-4-yl}amino)carbonyl]-3-methylbutyl}-1-methyl-1H-indole-2-carboxamide, and N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide.

Suitable expectorants include guaifenesin, guaiacolculfonate, ammonium chloride, potassium iodide, tyloxapol, antimony pentasulfide and the like.

Suitable vaccines include nasally inhaled influenza vaccines and the like.

Suitable macromolecules include proteins and large peptides, polysaccharides and oligosaccharides, DNA and RNA nucleic acid molecules and their analogs having therapeutic, prophylactic or diagnostic activities. Proteins can include growth factors, hormones, cytokines (e.g., chemokines), and antibodies. As used herein, antibodies can include: all types of immunoglobulins, e.g. IgG, IgM, IgA, IgE, IgD, etc., from any source, e.g. human, rodent, rabbit, cow, sheep, pig, dog, other mammals, chicken, other avian, aquatic animal species etc., monoclonal and polyclonal antibodies, single chain antibodies (including IgNAR (single-chain antibodies derived from sharks)), chimeric antibodies, bifunctional/bispecific antibodies, humanized antibodies, human antibodies, and complementary determining region (CDR)-grafted antibodies, that are specific for the target protein or fragments thereof, and also include antibody fragments, including Fab, Fab′, F(ab′)2, scFv, Fv, camelbodies, microantibodies, nanobodies, and small-modular immunopharmaceuticals (SMIPs). Nucleic acid molecules include DNA, e.g. encoding genes or gene fragments, or RNA, including mRNA, antisense molecules, such as antisense RNA, RNA molecules involved in RNA interference (RNAi), such as microRNA (miRNA), small interfering RNA (siRNA) and small hairpin RNA (shRNA), ribozymes or other molecules capable of inhibiting transcription and/or translation.

Suitable antihistamines include clemastine, asalastine, loratadine, fexofenadine and the like.

Suitable cough suppressants include benzonatate, benproperine, clobutinal, diphenhydramine, dextromethorphan, dibunate, fedrilate, glaucine, oxalamine, piperidione, opiods such as codeine and the like.

Suitable brochodilators include short-acting beta₂-agonists (SABAs), long-acting beta₂-agonists (LABA), long-acting muscarinic antagonist (LAMA), combinations of LABAs and LAMAs, methylxanthines, short-acting anticholinergic agents (may also be referred to as short-acting anti-muscarinic agents), long-acting bronchodilators, and the like.

Another suitable bronchodilator class is Muscarinic Antagonist-beta₂-agonist (MABA).

Suitable short-acting beta₂-agonists include albuterol, epinephrine, pirbuterol, levalbuterol, metaproteronol, maxair, and the like. A combination of a short activing beta₂-agonist and an anticholinergic is albuterol and ipatropium bromide (Combivent; Boehringer Ingelheim).

Examples of albuterol sulfate formulations (also called salbutamol) include Inspiryl (AstraZeneca Plc), Salbutamol SANDOZ (Sanofi-Aventis), Asmasal clickhaler (Vectura Group Plc.), Ventolin (GlaxoSmithKline Plc), Salbutamol GLAND (GlaxoSmithKline Plc), Airomir® (Teva Pharmaceutical Industries Ltd.), ProAir HFA (Teva Pharmaceutical Industries Ltd.), Salamol (Teva Pharmaceutical Industries Ltd.), Ipramol (Teva Pharmaceutical Industries Ltd), Albuterol sulfate TEVA (Teva Pharmaceutical Industries Ltd), and the like. Examples of epinephrine include Epinephine Mist KING (King Pharmaceuticals, Inc.), and the like. Examples of pirbuterol as pirbuterol acetate include Maxair® (Teva Pharmaceutical Industries Ltd.), and the like. Examples of levalbuterol include Xopenex® (Sepracor or Dainippon Sumitomo), and the like. Examples of metaproteronol formulations as metaproteronol sulfate include Alupent® (Boehringer Ingelheim GmbH), and the like.

Suitable LABAs include salmeterol, formoterol and isomers (e.g., arformoterol), clenbuterol, tulobuterol, vilanterol (GSK642444, also referred to Revolair™), indacaterol, carmoterol, isoproterenol, procaterol, bambuterol, milveterol, olodaterol, AZD3199 (AstraZeneca), and the like.

Examples of salmeterol formulations include salmeterol xinafoate as Serevent® (GlaxoSmithKline Plc), salmeterol as Inaspir (Laboratorios Almirall, S.A.), Advair® HFA (GlaxoSmithKline PLC), Advair Diskus® (GlaxoSmithKline PLC, Theravance Inc), Plusvent (Laboratorios Almirall, S.A.), VR315 (Novartis, Vectura Group PLC) and the like. Examples of formoterol and isomers (e.g., arformoterol) include Foster (Chiesi Farmaceutici S.p.A), Atimos (Chiesi Farmaceutici S.p.A, Nycomed Internaional Management), Flutiform® (Abbott Laboratories, SkyePharma PLC), MFF258 (Novartis AG), Formoterol clickhaler (Vectura Group PLC), Formoterol HFA (SkyePharma PLC), Oxis® (Astrazeneca PLC), Oxis pMDI (Astrazeneca), Foradil® Aerolizer (Novartis, Schering-Plough Corp, Merck), Foradil® Certihaler (Novartis, SkyePharma PLC), Symbicort® (AstraZeneca), VR632 (Novartis AG, Sandoz International GmbH), MFF258 (Merck & Co Inc, Novartis AG), Alvesco® Combo (Nycomed International Management GmbH, Sanofi-Aventis, Sepracor Inc), Mometasone furoate (Schering-Plough Corp), and the like. Examples of clenbuterol include Ventipulmin (Boehringer Ingelheim), and the like. Examples of tulobuterol include Hokunalin Tape (Abbott Japan Co., Ltd., Maruho Co., Ltd.), and the like. Examples of vilanterol include Revolair™ (GlaxoSmithKline PLC), GSK64244 (GlaxoSmithKline PLC), and the like. Examples of indacaterol include QAB149 (Novartis AG, SkyePharma PLC), QMF149 (Merck & Co Inc) and the like. Examples of carmoterol include CHF4226 (Chiese Farmaceutici S.p.A., Mitsubishi Tanabe Pharma Corporation), CHF5188 (Chiesi Farmaceutici S.p.A), and the like. Examples of isoproterenol sulfate include Aludrin (Boehringer Ingelheim GmbH) and the like. Examples of procaterol include Meptin clickhaler (Vectura Group PLC), and the like. Examples of bambuterol include Bambec (AstraZeneca PLC), and the like. Examples of milveterol include GSK159797C (GlaxoSmithKline PLC), TD3327 (Theravance Inc), and the like. Examples of olodaterol include BI1744CL (Boehringer Ingelheim GmbH) and the like. Other LABAs include Almirall-LAS100977 (Laboratorios Almirall, S.A.), and UK-503590 (Pfizer).

Examples of LAMAs include tiotroprium (Spiriva), trospium chloride, glycopyrrolate, aclidinium, ipratropium, darotropium, and the like.

Examples of tiotroprium formulations include Spiriva (Boehringer-Ingleheim, Pfizer), and the like. Examples of glycopyrrolate include Robinul® (Wyeth-Ayerst), Robinul® Forte (Wyeth-Ayerst), NVA237 (Novartis), and the like. Examples of aclidinium include Eklira® (Forest Labaoratories, Almirall), and the like. Examples of darotropium include GSK233705 (GlaxoSmithKline PLC). Examples of other LAMAs include BEA2180BR (Boehringer-Ingleheim), Ba 679 BR (Boehringer-Ingleheim), GSK573719 (GlaxoSmithKline PLC), GSK 160724 (GlaxoSmithKline PLC and Theravance), GSK704838 (GlaxoSmithKline PLC), QAT370 (Novartis). QAX028 (Novartis), AZD8683 (AstraZeneca), and TD-4208 (Theravance).

Examples of combinations of LABAs and LAMAs include indacaterol with glycopyrrolate, formoterol with glycopyrrolate, indacaterol with tiotropium, olodaterol and tiotropium, formoterol and tiotropium, vilanterol with a LAMA, and the like. Examples of combinations of formoterol with glycopyrrolate include PT003 (Pearl Therapeutics) and the like. Examples of combinations of olodaterol with tiotropium include BI11744 with Spirva (Boehringer Ingelheim) and the like. Examples of combinations of vilanterol with a LAMA include GSK573719 with GSK642444 (GlaxoSmithKline PLC), and the like.

Examples of combinations of indacaterol with glycopyrrolate include QVA149A (Novartis), and the like.

Examples of methylxanthine include aminophylline, ephedrine, theophylline, oxtriphylline, and the like.

Examples of aminophylline formulations include Aminophylline BOEHRINGER (Boehringer Ingelheim GmbH) and the like. Examples of ephedrine include Bronkaid® (Bayer AG), Broncholate (Sanofi-Aventis), Primatene (Wyeth), Tedral SA®, Marax (Pfizer Inc) and the like. Examples of theophylline include Euphyllin (Nycomed International Management GmbH), Theo-dur (Pfizer Inc, Teva Pharmacetuical Industries Ltd) and the like. Examples of oxtriphylline include Choledyl SA (Pfizer Inc) and the like.

Examples of short-acting anticholinergic agents include ipratropium bromide, and oxitropium bromide.

Examples of ipratropium bromide formulations include Atrovent®/Apovent/Inpratropio (Boehringer Ingelheim GmbH), Ipramol (Teva Pharmaceutical Industries Ltd) and the like. Examples of oxitropium bromide include Oxivent (Boehringer Ingelheim GmbH), and the like.

Selected therapeutics helpful for chronic maintenance of CF include antibiotics/macrolide antibiotics, bronchodilators, inhaled LABAs, and agents to promote MCC. Suitable examples of antibiotics/macrolide antibiotics include tobramycin, azithromycin, ciprofloxacin, colistin, aztreonam and the like. Another exemplary antibiotic/macrolide is levofloxacin. Suitable examples of bronchodilators include inhaled short-acting beta₂-agonists such as albuterol, and the like. Suitable examples of inhaled LABAs include salmeterol, formoterol, and the like. Suitable examples of agents to promote airway secretion clearance include Pulmozyme, DNase (dornase alfa, Genentech) hypertonic saline (HS), heparin, and the like. Selected therapeutics helpful for the treatment of CF include VX-770 (Vertex Pharmaceuticals) and amiloride.

Selected therapeutics helpful for the treatment of idiopathic pulmonary fibrosis (IPF) include Metelimumab (CAT-192) (TGF-beta 1 mAb inhibitor, Genzyme), Aerovant™ (AER001, pitrakinra) (Dual IL-13, IL-4 protein antagonist, Aerovance), Aeroderm™ (PEGylated Aerovant, Aerovance), microRNA, RNAi, and the like.

Antifibrotic agents are particularly useful for the treatment of idiopathic pulmonary fibrosis (IPF), such as pirfenidone (5-Methyl-1-phenyl-2-(1H)-pyridone) and Tacrolimus (FK506). Other antifibrotic agents include cyclosporin A, baicalein, ACE inhibitors, angiotensin receptor blockers, HMG-CoA reductase inhibitors, azathioprine, methotrexate, cyclophosphamide, TNF alpha blocking agents, TGF beta modulators (e.g. metelimumab (CAT-192), GC1008, alpha v beta 6 inhibitors, ALK5 inhibitors, hepatic growth factor (HGF), recombinant bone-morphogenic protein-7 (BMP-7), decorin, tyrosine-kinase inhibitors (e.g. Imatinib, Dasatinib, Nolitinib)), matrix-metalloproteases, inhibitors of tissue inhibitor of matrix metalloproteases (TIMP), vascular endothelial growth factor (VEGF) blockade (BIBF 1120), and the like.

Other therapeutic agents include, Meropenem (an anti infective therapeutic, for example, a bacterial), long acting corticosteroids (LAICS), the class of therapeutics known as MABAs (bifunctional muscarinic beta₂-agonist agonists), beclomethasone dipropionate (BDP)/formoterol (combination formulation), caffeine citrate (a citrate salt of caffeine) for short-term treatment of apnea of prematurity (lack of breathing in premature infants), surfactants for treatment of neonatal respiratory distress syndrome (RDS) (difficulty to breathe), the class of therapeutics know as caspase inhibitors (for example, for the treatment of Neonatal Brain injury), and the class of therapeutics known as Gamma secretase Modulators (for example, for the treatment of Alzheimer disease, etc.).

Examples of MABAs are AZD 2115 (AstraZeneca), GSK961081 (GlaxoSmithKline), and LAS190792 (Almirall).

The therapeutic agents mentioned herein are listed for illustrative purposes only, and one of ordinary skill will appreciate that any given therapeutic agent identified by a structural or functional class may be replaced with another therapeutic agent of the same structural or functional class.

If desired or indicated, the one or more other therapeutic agents described herein can be administered with a calcium salt formulation described herein. Calcium salt formulations (e.g., Formulations I, II, III, IV, and V) exhibit anti-pathogenic and/or anti-inflammatory effects at certain doses. If desired, they can be formulated to contain one or more additional therapeutic agent(s) as a co-formulation (e.g., two or more therapeutic agents in the same formulation or blended together). Alternatively, an additional therapeutic agent may be administered substantially concurrently with, prior to or subsequent to administration of the calcium salt formulation.

In a second aspect, the invention relates to methods of diagnosing an inflammation, infection and/or irritation of the respiratory tract in a subject. The subject may then be selected for therapy comprising a suitable calcium ion regimen described herein.

Subjects reporting with respiratory tract conditions may often receive unnecessary or misdirected medical treatment if the underlying condition is either misdiagnosed or undiagnosed by the treating physician. For example, viral respiratory tract infections or non-pathogenic irritations, e.g. caused by environmental agents (e.g. allergens, irritants) would not be suitable candidates for treatment with antibiotics, while bacterial infections would be. For viral respiratory tract infections treatment with anti-virals may be indicated, for irritations, anti-inflammatory treatment may be indicated. The diagnostic methods described herein include detection of certain biomarker profiles that allow a determination of the underlying condition, e.g. inflammation, irritation and/or infection. A physician, based on the biomarker profile obtained from the subject may be able to determine a suitable treatment regimen, potentially avoiding the administration of unnecessary and/or ineffective therapeutic agents, thereby making the treatment potentially more efficacious by targeting it to the specific underlying condition. Based on the biomarker profile, the physician may elect to treat the subject with a calcium ion dose regimen described herein. The physician may elect a suitable dose or amount of calcium ions, e.g. a high-, mid- or low calcium ion dose, as well as optionally co-administering one or more additional therapeutic agents (e.g. anti-inflammatory or anti-infectious agents) based on the biomarker profile obtained from the patient. The patient may present with or may be known to have a respiratory disease (e.g. a chronic airway disease or a pulmonary disease), such as asthma, airway hyper-responsiveness, seasonal allergic allergy, bronchiectasis, chronic bronchitis, emphysema, chronic obstructive pulmonary disease (COPD), cystic fibrosis (CF), and the like. Alternatively, the subject may not have any such respiratory disease and may experience an acute inflammation, irritation and/or infection independent of the aforementioned chronic respiratory diseases or conditions.

In some aspects, the invention relates to a method for diagnosing, selecting a patient for therapy, or monitoring efficacy of therapy of respiratory diseases (e.g. a chronic airway diseases and a pulmonary diseases), such as asthma, airway hyperresponsiveness, seasonal allergic allergy, bronchiectasis, chronic bronchitis, emphysema, chronic obstructive pulmonary disease, cystic fibrosis, and the like.

In other aspects, the invention relates to a method for diagnosing, selecting a patient for therapy, or monitoring efficacy of therapy of acute exacerbations of a respiratory disease (e.g. a chronic airway disease or a pulmonary disease), such as asthma, airway hyperresponsiveness, seasonal allergic allergy, bronchiectasis, chronic bronchitis, emphysema, chronic obstructive pulmonary disease, cystic fibrosis and the like.

In yet other aspects, the invention relates to a method for diagnosing, selecting a patient for therapy, or monitoring efficacy of therapy of a respiratory disease or respiratory condition, e.g. pulmonary parenchyal inflammatory/fibrotic conditions, such as idiopathic pulmonary fibrosis (IPF), pulmonary interstitial inflammatory conditions (e.g., sarcoidosis, allergic interstitial pneumonitis (e.g., Farmer's Lung)), fibrogenic dust interstitial diseases (e.g., asbestosis, silicosis, beryliosis), eosinophilic granulomatosis/histiocytosis X, collagen vascular diseases (e.g., rheumatoid arthritis, scleroderma, lupus), Wegner's granulomatosis, and the like.

In other aspects, the invention relates to a method for diagnosing, selecting a patient for therapy, or monitoring efficacy of therapy of a respiratory disease or respiratory condition associated with a pathogenic infectious (e.g. viral or bacterial) of the respiratory tract.

The methods of diagnosing comprise determining the absence, presence, relative amount, over- or underrepresentation, or fold-change of one or more biomarkers described herein relative to a control profile, wherein the absence, presence, relative amount, over- or underrepresentation of one or more biomarkers correlates with the absence or presence of an inflammation, irritation, and/or infection of the respiratory tract of a subject, and wherein the physician, based on the biomarker profile may determine i) if a patient is suitable for a calcium ion therapy described herein, ii) the suitable dose or dose range of calcium ions, iii) if so desired, the need for—or suitability of— one or more additional therapeutic agents, and/or iv) whether a therapy that is ongoing is effective or ineffective. A subject may be suitable for a calcium ion therapy described herein if the biomarker profile provided indicates the presence of an inflammation, irritation and/or infection. The therapy can comprise administering to the respiratory tract of a subject in need thereof an effective dose or amount of calcium ions and optionally co-administering one or more additional therapeutic agents.

The diagnostic methods described herein can be used to diagnose, select a patient for therapy, or monitor efficacy of therapy of acute or chronic inflammation and, in particular, inflammation that characterizes a number of respiratory diseases (e.g. chronic airway diseases and pulmonary diseases) and respiratory conditions including, asthma, airway hyperresponsiveness, seasonal allergic allergy, bronchiectasis, chronic bronchitis, emphysema, chronic obstructive pulmonary disease (COPD), cystic fibrosis (CF), pulmonary parenchyal inflammatory diseases/conditions, and the like.

Following the detection and/or determination of one or more biomarkers, one may compare the set of biomarkers providing a first biomarker profile from a subject suspected of having an inflammation, irritation, and/or infection with a statistically significant reference group of normal (healthy) subjects, who provide a suitable control profile. When compared to a control profile, the presence, absence, the under- or overrepresentation of certain biomarkers may indicate the presence of an inflammation, irritation and/or infection. Optionally, the first profile may also be compared to one or more suitable profiles derived from control subjects having a known inflammation, irritation, and/or infection. As will be appreciated, the suitable control profile(s) will not need to be generated each time a comparison is made to a first profile. Once suitable control biomarker profiles are determined, they can be stored, e.g. electronically, and may be provided for future use as reference profiles against one or more first profiles derived from a subject suspected of having an inflammation, irritation, and/or infection. Alternatively, when monitoring efficacy of treatment, one may obtain a first sample from the subject suspected of having an inflammation, irritation, and/or infection at the commencement of treatment, which represents the suitable control sample, and a second, third, fourth, etc. sample at certain time intervals during treatment, which are then compared to the first sample. Optionally, the second, third, fourth, etc. sample may also be compared to a suitable control sample obtained from normal (healthy) subjects. A change in biomarker profile toward closer resemblance of that at baseline would indicate that a given treatment is efficacious. If the biomarker profile does not significantly change in the second, third, fourth, etc. sample when compared to the first sample, e.g. if the biomarker profile indicates the continuous presence of an inflammation, irritation, and/or infection, such finding would indicate that the treatment regimen is not efficacious.

For example, a subject may be diagnosed with having an inflammation, irritation, and/or viral infection based on results obtained from a biomarker array contacted with a sample derived from the subject, wherein the biomarkers' absence, presence and/or concentration (e.g. under- or overrepresentation relative to a suitable control sample) in the sample are determined as described herein, and wherein the biomarker array comprises one or more, two or more, three or more, four or more, five or more, etc. biomarkers independently selected from the group consisting of (i) inflammation signature: Areg, Ccl2/MCP-1, Ccl7/MCP-3, Ccl17, Ccl20/MIP-3a, Cxcl1/KC, Cxcl2/MIP-2, Cxcl5/ENA78, Cxcl9, Cxcl10, Gpr81, IL-6, Ptgs2, and TNF; (ii) irritation signature: Adrb1, Aplnr, Bdnf, Birc5, Bmp6, Brca1, C8a, Ccl5, Ccl6, Ccr1, Ccr6, Ccr9, Ccrl1, Ccrl2, Clec7a, Cmtm5, Creb1, Cxcl13, Cxcr1, Cxcr4, Cxcr5, Fas1, Hspb1, Igfbp3, Il16, Il1r2, Il1rn, Kcna5, Lef1, Lep, Lif, Nos2, Per1, Pin, Proc, Pou2af1, Ppbp, Prl2c2, Rgs3, Tlr, Tir8, Tlr9, and Xcl1; and/or (iii) infection signature: Calb1, C, Ccl4. Ccl12. Csf2/GM-CSF, Egr1, Gem, Gusb, Hif1a. Ifngr2, Il1a, Il1b, Junb, Pmaip1, Serpina1a, Sod2, and Thbs1, and wherein the expression of one or more biomarkers may be increased or decreased by a factor of least 1.5, at least 2, at least 2.5, at least 3, at least 3.5, or at least 4 when compared to a suitable control profile.

For example, the subject may exhibit an inflammation if one or more biomarkers of the group consisting of Areg, Ccl2/MCP-1, Ccl7/MCP-3, Ccl17, Ccl20/MIP-3a, Cxcl1/KC, Cxcl2/MIP-2, Cxcl5/ENA78, Cxcl9, Cxcl10, IL-6, Ptgs2, and TNF are increased, and/or ifGpr81 is decreased when compared to a suitable control profile.

For example, the subject may exhibit an irritation if one or more biomarkers of the group consisting of Birc5, Brca1, Ccl6, Ccr1, Clec7a, Cxcl13, Cxcr1, Il1 r2, Il1rn, and Lif are increased, and/or if one or more biomarkers of the group consisting of Adrb1, Api1nr, Bdnf, Bmp6, C8a, Ccl5, Ccr6, Ccr9, Ccrl1, Ccrl2, Cmtm5, Creb1, Cxcr4, Cxcr5, Fas1, Hspb1, Igfbp3, Il16, Kcna5, Lef1, Lep, Nos2, Per1, Pin, Proc, Pou2af1, Ppbp, Prl2c2. Rgs3, Tlr1, Tlr8, Tlr9, and Xcl1 are decreased when compared to a suitable control profile.

For example, the subject may exhibit a viral infection if one or more biomarkers of the group consisting of Calb1, Ccl4, Ccl12, Csf2/GM-CSF, Egr1, Gem, Ifngr2, Il1a, Il1b, Junb and Thbs1 are increased, and/or if one or more biomarkers of the group consisting of Gusb, Hif1a, Pmaip1, Serpina1a, and Sod2, are decreased when compared to a suitable control profile.

It will be appreciated that a suitable biomarker array may consist of a number of the aforementioned biomarkers that indicate the underlying condition with reasonably high confidence. The degree of confidence required may be chosen according to standard practices, or may exceed standard practices. Generally, the higher the number of biomarkers on an array for a given indication, the higher the degree of confidence that a given indication is present. However, as will be appreciated, certain biomarkers when combined in low numbers may be fully sufficient to indicate an underlying condition, while others may need to be combined in larger numbers to confer the same degree of confidence. High confidence biomarkers might be those that change more significantly then others (e.g. by a factor of 3, 4, 5, or more), are more abundantly or more selectively expressed, or may be expressed more consistently among different subjects and/or different conditions. Other factors that may determine a high confidence biomarker may include relative high affinity interactions between the capturing agent or the visualizing agent and the biomarker, relative ease of isolation of the biomarker from the sample, relative stability of the biomarker, and the like, when compared to other biomarkers. One of skill in the art can determine the necessary and sufficient number of biomarkers on an array using only routine optimization.

A particularly preferred biomarker is IL-8. In a preferred embodiment, the one or more biomarker is selected from the group consisting of IL-8, IL-6, GM-CSF, and IL1-beta. Particularly preferred is a biomarker array that consist of i) IL-8, ii) IL-8 and IL-6, iii) IL-8 and GM-CSF, iv) IL-8 and IL1-beta, v) IL-8, IL-6, and GM-CSF or IL1-beta, or vi) IL-8, IL-6, GM-CSF and IL1-beta. Other preferred biomarker arrays may consist of or may consist essentially of i) one or more of IL-8, IL-6, GM-CSF and IL1-beta, and ii) one or more of Areg, Ccl2/MCP-1, Ccl7/MCP-3, Ccl17, Ccl20/MIP-3a, Cxcl2/MIP-2, Cxcl5/ENA78, Cxcl9, Cxcl10, Gpr81, Ptgs2, and TNF; and/or iii) one or more of Adrb1, Aplnr, Bdnf, Birc5, Bmp6, Brca1, C8a, Ccl5, Ccl6, Ccr1, Ccr6, Ccr9, Ccrl1, Ccrl2, Clec7a, Cmtm5, Creb1, Cxcl13, Cxcr1, Cxcr4, Cxcr5, Fas1, Hspb1, Igfbp3, Il16, Il1r2, Il1rn, Kcna5, Lef1, Lep, Lif, Nos2, Per1, Pln, Proc, Pou2af1, Ppbp, Prl2c2, Rgs3, Tlr1, Tlr8, Tlr9, and Xcl1; and/or vi) one or more of Calb1, Ccl4, Ccl12, Egr1, Gem, Gusbh. Hif1a, Ifngr2, Il1a, Junb, Pmaip1, Serpina1a, Sod2, and Thbs1. Additionally preferred biomarkers suitable for an array are one or more biomarkers, two or more biomarkers, three or more biomarkers, four or more biomarkers, five or more biomarkers, or six or more biomarkers selected from the group consisting of: TNF-alpha, IL-8, IL-6, IL-2, IL1-beta, INF-gamma, GM-CSF, MMP-1 and MMP-9.

Respiratory conditions include temporary inflammatory conditions (e.g. caused by an environmental insult, such as exposure to an irritant) and temporary infectious conditions (e.g. caused by exposure to a pathogen). Respiratory diseases (e.g. chronic airway diseases and pulmonary diseases) include long-term or chronic diseases with underlying inflammation/irritation, such as asthma, airway hyperresponsiveness, seasonal allergic allergy, brochiectasis, chronic bronchitis, emphysema, chronic obstructive pulmonary disease, cystic fibrosis, pulmonary parenchyl inflammatory conditions, and the like.

Irritants that can cause environmental insults include environmental allergens, irritants, e.g., aeroallergens and airborne particulates, and the like. Irritation may be caused by an irritant that is independently tobacco smoke, ozone, fine particulate dust, dust mite, pet dander, cockroach allergen, mold, pollen, or a volatile organic compound. The fine particulate dust may be e.g. Diesel exhaust, silica (SiO₂), asbestos, and the like. The volatile organic compound can be e.g. benzene, chlorobenzene, styrene, and the like. The irritation can be caused by an agent for bronchial-provocation testing (BPT), such as, e.g., methacholine, histamine, dry mannitol, dextrose, hypertonic saline, and the like.

Irritation may be caused by an irritant that is an aeroallergen. Aeroallergens can be soluble or particulate. Aeroallergens (either soluble or particulate) include, but are not limited to, pollen (such as, from trees, e.g. birch (Betula), alder (Alnus), cedar (Cedrus), hazel (Corylus), hornbeam (Carpinus), horse chestnut (Aesculus), willow (Salix), poplar (Populus), plane (Platanus), linden/lime (Tilia), juniper, maple, elm, oak, pine, mulberry, ash, walnut, sweet gum, sycamore and olive (Olea); grasses, e.g. Poaceae family, ryegrass (Lolium sp.), timothy grass (Phleum pratense), Kentucky bluegrass, fescues, orchard grass, redtop grass, Johnson grass, and vernal grass; weeds, e.g. ragweed (Ambrosia), plantain (Plantago), nettle/parietaria (Urticaceae), mugwort (Artemisia), Fat hen (Chenopodium), sage, lambs quarter, English plantain, yellow dock, sheep sorrel, pigweed and sorrel/dock (Rumex)), spores (e.g. released by fungi or plants, including mold spores (indoor: Aspergillus, Penicillium, Rhizopus and Stachybotrys) and other spores (including outdoor mold), such as e.g. Altemaria, Cladosporium, Ascospores, Basidiospores, Epicoccum, Pithomyces, Sporangiospores, Zygospores, Aeciospores, Urediospores, Teliospores, Oospores, Carpospores, Tetraspores, Meiospores. Microspores. Megaspores, Macrospores, Mitospores, Conidiospores, spores from Rusts, Botrytis, Cercospora, Curvularia, Drechslera, Oidium, Polythrincium, Stemphylium, and Torula), other indoor aeroallergens, including dust mite ((Dermatophagoides, pternonyssinus and D. farinae), cockroaches, animal dander (e.g. cat, dog, horse, cow, sheep, goat, rabbit, gerbil, hamster, guinea pig, rat and mice dander), aerosolized occupational allergens (e.g. grain mite, grain dust, fungal amylase, pancreatin, papain, pepsin, diisocyanates, pthalic/acid anhydride, ethylene diamine, azodicarbonamide, methyl methacrylate, halogenated platinum salts, cobalt, chromium, nickel). Clinically significant aeroallergens include proteins or glycoproteins with a molecular weight of 10,000 to 60,000 Daltons. Ragweed is about 20 microns in diameter; tree pollen is 20-60 microns; and grass pollen is 30-40 microns. Aeroallergens may act in conjunction with other irritants and pollutants, such as, e.g. carbon monoxide, lead, nitrogen dioxide, ozone, sulfur dioxide, particulate matter.

Viruses causing a respiratory tract infection include influenza virus, parainfluenza virus, respiratory syncytial virus, rhinovirus, adenovirus, metapneumovirus, coxsackie virus, echo virus, corona virus, herpes virus, cytomegalovirus, and the like.

Bacteria causing a respiratory tract infection include Streptococcus pneumoniae, which is commonly referred to as pneumococcus, Staphylococcus aureus, Burkholderis ssp., Streptococcus agalactiae, Haemophilus influenzae. Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Mycobacterium tuberculosis, Bordetella pertussis, and the like.

Fungi causing a respiratory tract infection include Histoplasma capsulatum, Cryptococcus neoformans, Pneumocvstis jiroveci, Coccidioides immitis, and the like.

Parasites causing a respiratory tract infection include Toxoplasma gondii, Strongyloides stercoralis, and the like.

Suitable biomarkers for diagnosing an inflammation, irritation, or infection, include (i) for inflammation: Areg, Ccl2/MCP-1, Ccl7/MCP-3, Ccl17, Ccl20/MIP-3a, Cxcl1/KC, Cxcl2/MIP-2, Cxcl5/ENA78, Cxcl9, Cxcl10, Gpr81, IL-6, Ptgs2, and TNF (inflammation signature); (ii) for irritation: Adrb1, Aplnr, Bdnf, Birc5, Bmp6, Brca1, C8a, Ccl5, Ccl6, Ccr1, Ccr6, Ccr9, Ccrl1, Ccrl2, Clec7a, Cmtm5, Creb1, Cxcl13, Cxcr1, Cxcr4, Cxcr5, Fas1, Hspb1, Igfbp3, Il16, Il1r2, Il1rn, Kcna5, Lef1, Lep, Lif, Nos2, Per1, Pin, Proc, Pou2af1, Ppbp, Prl2c2, Rgs3, Tlr1, Tlr8, Tlr9, and Xcl1 (irritation signature); and (iii) for viral infection: Calb1, Ccl14, Ccl12, Csf2/GM-CSF, Egr1, Gem, Gusb, Hif1a, Ifngr2, Il1a, Il1b, Junb, Pmaip1, Serpina1a, Sod2, Thbs1 (infection signature).

The expression of one or more biomarkers may be increased or decreased by a factor of least 1.5, at least 2, at least 2.5, at least 3, at least 3.5, or at least 4 when compared to a suitable control profile.

For example, the inflammation signature group consists of genes that are upregulated (increased): Areg, Ccl2/MCP-1, Ccl7/MCP-3, Ccl17, Ccl20/MIP-3a, Cxcl1/KC, Cxcl2/MIP-2, Cxcl5/ENA78, Cxcl9, Cxcl10, IL-6, Ptgs2, and TNF, as well as one gene that is downregulated (decreased): Gpr81, and one or more of these biomarkers may independently be selected and compared to a suitable control profile.

The irritation signature group consists of genes that are upregulated (increased): Birc5, Brca1, Ccl6, Ccr1, Clec7a, Cxcl13, Cxcr1, Il1r2, Il1rn, and Lif, as well as genes that are downregulated (decreased): Adrb1. Aplnr, Bdnf, Bmp6, C8a. Ccl5. Ccr6, Ccr9, Ccrl1, Ccrl2, Cmtm5, Creb1, Cxcr4, Cxcr5, Fas1, Hspb1, Igfbp3, Il16, Kcna5, Lef1, Lep, Nos2, Per1, Pin, Proc, Pou2af1, Ppbp, Prl2c2, Rgs3, Tlr1, Tlr8, Tlr9, and Xcl1, and one or more of these biomarkers may independently be selected and compared to a suitable control profile.

The infection signature group consists of genes that are upregulated (increased): Calb1, Ccl4, Ccl12, Csf2/GM-CSF, Egr1, Gem, Ifngr2, Il1a, Il1b, Junb, and Thbs1, as well as genes that are downregulated (decreased): Gusb, Hif1a, Pmaip1, Serpina1a, and Sod2, and one or more of these biomarkers may independently be selected and compared to a suitable control profile.

The profile of expression may include two or more, three or more, four or more, five or more, six or more, seven or more, eight or more, nine or more or ten or more biomarkers. The biomarkers may be a protein, a polypeptide, a peptide fragment, a nucleic acid, an mRNA, microRNA or a combination thereof.

The sample comprising the one or more biomarkers can be, for example, exhaled breath condensate, sputum, bronchoalveolar lavage (BAL) fluid, nasal lavage, bronchial or nasal biopsy, epithelial brushings, whole blood, serum, plasma, lymph fluid, cerebrospinal fluid, saliva, urine, mucus, and the like.

Relative levels of the one or more biomarkers in the sample can be determined using detection methods well known in the art, for example, as described in US Publication No. 2006-0094056 (PCT Publication No. WO 2005/029091 “METHOD OF USING CYTOKINE ASSAYS TO DIAGNOSE, TREAT, AND EVALUATE INFLAMMATORY AND AUTOIMMUNE DISEASES”) and US 2011-0117107 (WO 2012/074577 “COMPOSITIONS AND METHODS FOR DETECTION AND MANAGEMENT OF MALARIA”). Useful assays include, but are not limited to, immunoassays, mass spectroscopy, PCR, DNA arrays, and restriction fragment length polymorphism (RFLP) analysis.

A protein array can include probes suitable for detection of protein biomarkers, for example, antibodies, specific ligands, hetero- or homodimerization protein partners, fusion proteins or fragments thereof. Exemplary methods for determining the expression of protein biomarkers include, for example, immunoassays.

Examples of immunoassays are enzyme immune assay (EIA), enzyme-linked inmmunosorbent assays (ELISAs), enzyme multiplied immunoassay (EMIT), radio-immunoassays (RIA), radioimmune precipitation assays (RIPA), Farr assay, immunobead capture assays, Western blotting, dot blotting, gel-shift assays, flow cytometry (fluorescent activated cell sorting (FACS)), immunofluorescent microscopy, protein arrays, multiplexed bead arrays, magnetic capture, in vivo imaging, fluorescence resonance energy transfer (FRET), fluorescence polarization immunoassay (FPIA), fluorescence recovery/localization after photobleaching (FRAP/FLAP), and combinations thereof.

Generally, immunoassays involve contacting a sample with a capturing agent (e.g. an antibody or antibody fragment) capable of interacting with a recognition site (e.g. an antigen) present on a biomarker under conditions effective to allow the formation of immunocomplexes.

Immunoassays may further comprise a step wherein the capturing agent is bound to or is capable of binding to a solid support (e.g., tube, well, bead, or cell) to capture the biomarker protein of interest from a sample, optionally combined with a method of detecting the biomarker protein or capturing agent specific for the biomarker protein on the support. Examples of such immunoassays include radioimmunoassay (RIA), enzyme-linked immunosorbent assay (ELISA), flow cytometry, protein array, multiplexed bead assay, and magnetic capture.

Protein arrays are solid-phase ligand binding assay systems using immobilized proteins on surfaces which include glass, membranes, microtiter wells, mass spectrometer plates, and beads or other particles. The assays can be highly parallel (multiplexed) and often miniaturized (microarrays, protein chips).

Capture arrays may also form the basis of diagnostic chips and arrays for expression profiling. They employ high affinity capture reagents, such as antibodies, Fab and scFv fragments, single domains, engineered scaffolds, peptides or nucleic acid aptamers, to bind and detect specific target ligands in high throughput manner.

Single-stranded nucleic acid aptamers that bind protein ligands with high specificity and affinity are also used in arrays. Aptamers can be selected from libraries of oligonucleotides e.g. by the Selex™ procedure and their interaction with protein can be enhanced by covalent attachment, e.g. through incorporation of brominated deoxyuridine and UV-activated crosslinking (photoaptamers) on photoaptamer arrays. Universal fluorescent protein stains can be used to detect binding.

Molecular imprinting technology involves the use of peptides as templates to generate structurally complementary, sequence-specific cavities in a polymerizable matrix; the cavities can then specifically capture (denatured) proteins that have the appropriate primary amino acid sequence.

ProteinChip® arrays (Ciphergen, Fremont, Calif.) employ solid phase chromatographic surfaces that bind proteins with similar characteristics of charge or hydrophobicity and SELDI-TOF mass spectrometry to detect the captured proteins.

A gene array can include probes (e.g. oligonucleotides or primers) suitable for detection of nucleic acid biomarkers. Exemplary methods for determining the expression of nucleic acid biomarkers include, for example, quantitative polymerase chain reaction (qPCR), real-time PCR (rtPCR), DNA microarray, RNA array, Northern blot and combinations thereof.

A DNA or oligonucleotide microarray consists of an arrayed series of a plurality of microscopic spots of oligonucleotides, each containing a specific oligonucleotide sequence. The specific oligonucleotide sequence can be a short section of a gene or other oligonucleotide element that are used as probes to hybridize a cDNA or cRNA sample under high-stringency conditions. Probe-target hybridization is usually detected and quantified by fluorescence-based detection of fluorophore-labeled targets to determine relative abundance of nucleic acid sequences in the target. The probes are typically attached to a solid surface by a covalent bond to a chemical matrix. The solid surface can be e.g. glass or a silicon chip or microscopic beads. The oligonucleotide can be RNA for expression profiling, DNA for comparative hybridization, or DNA/RNA bound to a particular protein which is immunoprecipitated (ChIP-on-chip).

For example, total RNA can be isolated by guanidinium thiocyanate-phenol-chloroform extraction. The purified RNA may be analyzed for quality (e.g., by capillary electrophoresis) and quantity (e.g., by using a NANODROP spectrometer, ThermoFisher Scientific, Waltham, Mass.). The RNA is reverse transcribed into DNA with either polyT primers or random primers. The DNA products may be optionally amplified by PCR. A label is added to the amplification product either in the RT step or in an additional step after amplification. The label can be a fluorescent label or a radioactive label. The labeled DNA products are then hybridized to the microarray. The microarray is then washed and scanned. The expression level of the biomarker gene of interest is determined based on the hybridization result using methods well known in the art.

Presence/absence or concentration of the one or more biomarkers may be determined using a kit provided herein. The kit may include an array of one or more biomarker capturing agents, e.g. provided as probes, primers, antibodies and the like, that are optionally immobilized on a substrate, e.g. a slide, a well, a tube, and the like. Suitable biomarkers for diagnosing an inflammation, irritation, or infection using the kit provided herein, include (i) for inflammation: Areg, Ccl2/MCP-1, Ccl7/MCP-3, Ccl17, Ccl20/MIP-3a, Cxcl1/KC, Cxcl2/MIP-2, Cxcl5/ENA78, Cxcl9, Cxcl10, Gpr81, IL-6, Ptgs2, and TNF (inflammation signature); (ii) for irritation: Adrb1, Aplnr, Bdnf, Birc5, Bmp6, Brca1, C8a, Ccl15, Ccl6, Ccr1, Ccr6, Ccr9, Ccr11, Ccrl2, Clec7a, Cmtm5, Creb1, Cxcl13, Cxcr1, Cxcr4, Cxcr5, Fas1, Hspb1, Igfbp3, Il16, Il1r2, Il1rn, Kcna5, Lef1, Lep, Lif, Nos2, Per1, Pin, Proc, Pou2af1, Ppbp, Prl2c2, Rgs3, Tlr1, Tlr8, Tlr9, and Xcl1 (irritation signature); and (iii) for viral infection: Calb1, Ccl4, Ccl12, Csf2/GM-CSF, Egr1, Gem, Gusb, Hif1a, Ifngr2, Il1a, Il1b, Junb, Pmaip1, Serpina1a, Sod2, Thbs1 (infection signature). A particularly preferred biomarker for use in the kit is IL-8. In a preferred embodiment, the one or more biomarker for use in the kit is selected from the group consisting of IL-8, IL-6, GM-CSF, and IL1-beta. Particularly preferred for use in the kit is a biomarker array that consist of i) IL-8, ii) IL-8 and IL-6, iii) IL-8 and GM-CSF, iv) IL-8 and IL1-beta, v) IL-8, IL-6, and GM-CSF or IL1-beta, or vi) IL-8, IL-6, GM-CSF and IL1-beta. Other preferred biomarker arrays for use in the kit may consist of or may consist essentially of i) one or more of IL-8, IL-6, GM-CSF and IL1-beta, and ii) one or more of Areg, Ccl2/MCP-1, Ccl7/MCP-3, Ccl17, Ccl20/MIP-3a, Cxcl2/MIP-2, Cxcl5/ENA78, Cxcl9, Cxcl10, Gpr81, Ptgs2, and TNF; and/or iii) one or more of Adrb1, Aplnr, Bdnf, Birc5, Bmp6, Brca1, C8a, Ccl5, Ccl6, Ccr1, Ccr6, Ccr9, Ccr11, Ccrl2, Clec7a, Cmtm5. Creb1, Cxcl13, Cxcr1, Cxcr4, Cxcr5. Fas1, Hspb1, Igfbp3, Il16, Il1r2, Il1rn, Kcna5, Lef1, Lep, Lif, Nos2, Per1, Pin, Proc, Pou2af1, Ppbp, Prl2c2, Rgs3, Tlr1, Tlr8, Tlr9, and Xcl1; and/or vi) one or more of Calb1, Ccl4, Ccl12, Egr1, Gem, Gusb, Hif1a, Ifngr2, Il1a, Junb, Pmaip1, Serpina1a, Sod2, and Thbs1. Additionally preferred biomarkers suitable for an array for use in the kit are one or more biomarkers, two or more biomarkers, three or more biomarkers, four or more biomarkers, five or more biomarkers, or six or more biomarkers selected from the group consisting of: TNF-alpha, IL-8, IL-6, IL-2, IL1-beta, INF-gamma, GM-CSF, MMP-1 and MMP-9. The kit may contain solutions, preservatives, sterilizing agents and/or tools (e.g. syringes, scalpels, swabs, tubes, containers and the like) suitable for obtaining a sample from the subject and/or for obtaining isolated, or partially isolated biomarkers (e.g. biomarker proteins, biomarker RNA, and the like) that may be contacted with the biomarker capturing agents. Alternatively, the isolated samples or biomarkers may be analyzed using specialized equipment not provided with the kit. The kit may comprise agents suitable for amplification of the biomarker signal, e.g. PCR reagents, enzymes, buffers, colorimetric agents, radio- or fluorescence labels, and the like. The kit may comprise reference protocols, instructions how to use the kit, reference biomarker profiles (e.g. suitable control profiles) and other tools for biomarker analysis, which may be provided electronically (e.g. via website access) or as hardcopies supplied with the kit.

In another aspect, the invention relates to methods for screening a test agent for efficacy in controlling inflammation of the respiratory tract associated with infection or irritation. The method may include the steps of selecting a suitable model of inflammation of the respiratory tract, administering the test agent to the model, obtaining a sample from the model after the test agent has been administered, analyzing the sample for the expression of one or more biomarkers of inflammation, wherein when the expression of one or more biomarkers selected from the group consisting of Areg, Ccl2/MCP-1, Cc7/MCP-3, Ccl7, Ccl20/MIP-3a, Cxcl1/KC, Cxcl2/MIP-2, Cxcl5/ENA78, Cxcl9, Cxcl10, IL-6, Ptgs2, and TNF, is decreased relative to a suitable control sample, or when the expression of Gpr81 is increased relative to a suitable control sample, the test agent exhibits efficacy in controlling inflammation of the respiratory tract. The model may be an in vitro cell/tissue culture model or an in vivo animal model (e.g., a whole animal model).

Optionally, the method may further include a step of modulating inflammation with one or more agents. Inflammation may be modulated by such agents as, for example, 2-[(aminocarbonyl)amino]-5-[4-fluorophenyl]-3-thiophenecarboxamide (TPCA-1); doxycycline; NR58-3.14.3; spiropiperidine; N-(6-chloro-9H-beta-carbolin-8-yl) nicotinamide (PS-1145); N-(6-chloro-7-methoxy-9H-beta-carbolin-8-yl)-2-methyl-nicotinamide (ML120B); N-acetylcysteine (NAC); antagonist anti-CCR2 (CCR2-05) monoclonal antibody; gamma-tocopherol; 2-cyano-3,12-dioxoolean-1,9-dien-28-oic acid (CDDO); 15-deoxy-delta(12,14)-prostaglandin J(2) (15d-PGJ(2)); GRP blocking agent 77427; GRP blocking antibody 2A11; IKK2 inhibitor (IMD-0354); GSK-3 inhibitor 3-(2,4-dichlorophenyl)-4-(1-methyl-1H-indol-3-yl)-1H-pyrrole-2,5-dione (SB216763); dehydroevodiamine; evodiamine; rutaecarpine; 5alpha-reductase inhibitor finasteride; cordycepin; Nox2 inhibitors, fluoxetine; chymase inhibitor 2-[4-(5-fluoro-3-methylbenzo[b]thiophen-2-yl)sulfonamido-3-methanesulfonylphenyl]thiazole-4-carboxylic acid (TY-51469); TNF-alpha converting enzyme (TACE) and matrix metalloproteinases (MMPs) dual inhibitors: PKF242-484, PKF241-466; CXCR4 antagonist AMD3100; inhibitor of p44/42 MAPK U0126; IKK-selective inhibitors: PS-1145 [N-(6-chloro-9H-beta-carbolin-8-ly) nicotinamide], ML120B [N-(6-chloro-7-methoxy-9H-beta-carbolin-8-yl)-2-methyl-nicotinamide]; artemisinin; proteasome inhibitors: pyrrolidine dithiocarbamate [PDTC], MG132, PS-341 (bortezomib); bindarit, thromboxane A(2) synthase inhibitor ozagrel; aminopeptidase N inhibitor actinonin; NF-kappa B inhibitor IKK-NBD; p38 MAP kinase inhibitors: SB 203580, SB 202190; neutrophil elastase inhibitor Sivelestat; quercetin (3,3′,4′,5,7-pentahydroxyflavone); N,N-dimethylsphingosine; phosphodiesterase inhibitor pentoxifylline; PKA inhibitor H-89; anti-CCR2-blocking monoclonal antibody MC21; IkappaB-alpha phosphorylation inhibitor BAY 11-7082; alpha-1-antitrypsin; and synthetic metalloprotease inhibitor (RS113456).

In a third aspect, the invention relates to methods for modulating Toll-like receptors (TLR) signaling. The methods comprise contacting a TLR-expressing cell with mono- or divalent metal cation or salts thereof in an amount sufficient to modulate TLR signaling, e.g. signaling through one or more of TLR1, TLR2. TLR3, TLR4, TLR5, TLR6, TLR7, TLR8, TLR9, TLR10, and combinations thereof.

Toll-like receptors (TLRs) are present on many cells of the immune system and have been shown to be involved in the innate immune response (Hornung, V. et al, (2002) J. Immunol. 168:4531-4537). In vertebrates, this family consists of ten proteins called TLR1 to TLR10, which are known to recognize pathogen associated molecular patterns (PAMPs) from bacteria, fungi, parasites, and viruses (Poltorak, a. et al. (1998) Science 282:2085-2088; Underhill, D. M., et al. (1999) Nature 401:811-815; Hayashi, F. et. al (2001) Nature 410:1099-1103; Zhang, D. et al. (2004) Science 303:1522-1526; Meier, A. et al. (2003) Cell. Microbiol. 5:561-570; Campos, M. A. et al. (2001) J. Immunol. 167: 416-423; Hoebe, K. et al. (2003) Nature 424: 743-748; Lund, J. (2003) J. Exp. Med. 198:513-520; Heil, F. et al. (2004) Science 303:1526-1529; Diebold, S. S., et al. (2004) Science 303:1529-1531; Hornung, V. et al. (2004) J. Immunol. 173:5935-5943). TLRs are a key means by which mammals recognize and mount an immune response to foreign molecules and also provide a means by which the innate and adaptive immune responses are linked (Akira, S. et al. (2001) Nature Immunol. 2:675-680; Medzhitov, R. (2001) Nature Rev. Immunol. 1:135-145). Some TLRs are located on the cell surface to detect and initiate a response to extracellular pathogens and other TLRs are located inside the cell to detect and initiate a response to intracellular pathogens. Of the ten mammalian TLRs, TLR3, 7, 8, and 9 are known to localize in endosomes inside the cell and recognize nucleic acids (DNA and RNA) and small molecules such as nucleosides and nucleic acid metabolites. TLR3 and TLR9 are known to recognize nucleic acid such as dsRNA and unmethylated CpG dinucleotide present in viral and bacterial and synthetic DNA, respectively.

Provided herein are methods to modulate TLR signaling. The methods comprise contacting a TLR-expressing cell with mono- or divalent metal cation or salts thereof in an amount sufficient to modulate TLR signaling, e.g. signaling through one or more of TLR1, TLR2, TLR3, TLR4, TLR5, TLR6, TLR7, TLR8, TLR9, TLR10, and combinations thereof. For example, modulation may affect one or more heterodimers, such as TLR1/2 or TLR2/6 and/or one or more homodimers. Modulation may, for example, be achieved in TLR1/2 and TLR2/6 as well as TLR2, TLR3, TLR4, TLR5, TLR7, TLR8, and/or TLR9. Modulation includes agonistic and antagonistic modulation. The mono- or divalent metal cations or salts thereof may further be combined with one or more additional TLR modulators, such as those set forth in Table 1. By modulating TLR signaling immune responses can be generated that are for example substantially muted, primarily Th1 driven, primarily Th2 driven, or Th1/Th2 balanced. Modulating the immune response through TLR signaling offers the opportunity to use the immune system to treat and prevent a variety of diseases without triggering an uncontrolled stimulation of the immune system through TLRs, which may exacerbate certain diseases.

T helper (Th) cells involved in cell-mediated functions such as delayed-type hypersensitivity and activation of cytotoxic T lymphocytes (CTLs) are Th1 cells, whereas the Th cells involved as helper cells for B-cell activation are Th2 cells. The type of immune response is influenced by the cytokines and chemokines produced in response to antigen exposure. Cytokines provide a means for controlling the immune response by affecting the balance of T helper 1 (Th1) and T helper 2 (Th2) cells, which directly affects the type of immune response that occurs. If the balance is toward higher numbers of Th1 cells, then a cell-mediated immune response occurs, which includes activation of cytotoxic T cells (e.g. CTLs). When the balance is toward higher numbers of Th2 cells, then a humoral or antibody immune response occurs. Each of these immune responses results in a different set of cytokines being secreted from Th1 and Th2 cells.

Th1 cells are involved in the body's innate response to antigen (e.g. viral infections, intracellular pathogens, and tumor cells). The initial response to an antigen can be the secretion of IL-12 from antigen presenting cells (e.g. activated macrophages and dendritic cells) and the concomitant activation of Th1 cells. The result of activating Th1 cells is a secretion of certain cytokines (e.g. IL-2, IFN-gamma and other cytokines) and a concomitant activation of antigen-specific CTLs. Th2 cells are known to be activated in response to bacteria, parasites, antigens, and allergens and may mediate the body's adaptive immune response (e.g. IgM and IgG production and eosinophil activation) through the secretion of certain cytokines (e.g. IL-3, IL-4, IL-5, IL-6, IL-9, IL-10, IL-13 and other cytokines) and chemokines. Secretion of certain of these cytokines may result in B-cell proliferation and an increase in antibody production.

Certain cytokines can stimulate or inhibit the release of other cytokines, e.g. IL-10 inhibits IFN-gamma secretion from Th1 cells and IL-12 from dendritic cells. The balance between Th1 and Th2 cells and the cytokines and chemokines released in response to selected stimulus can have an important role in how the body's immune system responds to disease. For example, IFN-alpha may inhibit hepatitis C, and MIP-1 alpha and MIP-1 beta (also known as CCL3 and CCL4 respectively) may inhibit HIV-1 infection. Optimal balancing of the Th1/Th2 immune response presents the opportunity to use the immune system to treat and prevent a variety of diseases.

TLRs have been shown to play a role in the pathogenesis of many diseases, including autoimmunity, infectious disease and inflammation (Papadimitraki et al. (2007) J. Autoimmun. 29: 310-318; Sun et al. (2007) Inflam Allergy Drug Targets 6:223-235; Diebold (2008) Adv Drug Deliv Rev 60:813-823; Cook, D. N. et al. (2004) Nature Immunol 5:975-979; Tse and Homer (2008) Semin Immunopathol 30:53-62; Tobias & Curtiss (2008) Semin Immunopathol 30:23-27; Ropert et al. (2008) Semin Immunopathol 30:41-51; Lee et al. (2008) Semin Immunopathol 30:3-9; Gao et al. (2008) Semin Immunopathol 30:29-40; Vijay-Kumar et al. (2008) Semin Immunopathol 30:11-21). TLR agonists and antagonists have been investigated extensively for their utility in balancing of the Th1/Th2 immune response, as immune modulatory agents and for their use alone or as adjuvants in immunotherapy to treat diseases or conditions such as allergy, asthma, autoimmunity, inflammatory diseases, cancer, and infectious disease (Marshak-Rothstein A, Nat Rev Immunol (2006) 6:823-35). While activation of TLRs is involved in mounting an immune response, an uncontrolled stimulation of the immune system through TLRs may exacerbate certain diseases, e.g. in immune compromised subjects. Therefore, a careful calibration of the immune response is important to achieve effective treatment or disease management.

Provided herein are methods for modulating TLR signaling comprising contacting a TLR-expressing cell with mono- or divalent metal cation or salts thereof in an amount sufficient to modulate TLR signaling through one or more of TLR1, TLR2, TLR3, TLR4, TLR5, TLR6, TLR7, TLR8, TLR9, TLR10, and combinations thereof. Preferably, the modulation achieved when using mono- or divalent metal cations or salts thereof is complemented with one or more additional TLR agonists or antagonists, e.g. those as set forth in Table 1 and others known in the art or being developed in the future.

TABLE 1
Exemplary TLR agonists and antagonists.

| Company | Agent | Target | Indication |
|---|---|---|---|
| Anadys/Novartis | ANA975 | TLR7 agonist | HCV |
| | (Isotorabine) | | |
| | ANA773 | TLR7 agonist | cancer |
| Cbio | Cpn10 (heat-shock | inhibits TLR | rheumatoid arthritis, psoriasis |
| | protein 10) | responses | and multiple sclerosis |
| Clinquest | CQ-07001 | protein TLR3 | |
| | | agonist | |
| Coley/Pfizer | PF-3512676 | TLR9 agonist | non-small cell carcinoma, |
| | (CpG7909)/ | | renal cell carcinoma, |
| | | | melanoma and cutaneous T- |
| | | | cell lymphoma |
| Coley/GSK, | Vaximmune | TLR9 agonist | vaccine adjuvant |
| Novartis | (CpG7909) | | |
| Coley/ | AVE0675 | TLR9 agonist | asthma/allergic rhinitis |
| SanofiAventis | | | |
| Dynavax | Heplislav | hepatitis B surface | |
| Technologies | | antigen vaccine | |
| | | containing TLR9 | |
| | | agonist | |
| | Tolamba | ragweed allergen | seasonal allergic rhinitis |
| | | therapeutic vaccine | |
| | | containing TLR9 | |
| | | agonist | |
| | SD-101 | TLR9 agonist | HepC virus infection |
| | IRS-954 | TLR7 and 9 | Systemic lupus |
| | | antagonist, | erythematosus (SEL) |
| | | interferon alpha | |
| | | antagonist | |
| Eisai | Eritoran (E5564) | LPS lipid A | Sepsis |
| | | analogue TLR4 | |
| | | antagonist | |
| GSK | AS04 | MPL, TLR4 agonist | adjuvant in multiple vaccines |
| | CRX-675 and CRX-527 | intranasal TLR4 | allergic rhinitis |
| | | agonists | |
| Hemispherx | Ampligen | TLR3 agonist | chronic fatigue syndrome, |
| Biopharma | poly(I): poly(C12U) | | HIV |
| Idera | IMO-2055 | TLR9 agonist | renal cell carcinoma |
| | Amplivax | TLR9 agonist | adjuvant in HIV-1 vaccine |
| | IMO-2125 | TLR9 agonist | HepC vims infection |
| | IMO-3100 | TLR7, 8, 9 | Systemic lupus |
| | | antagonist | erythematosus (SEL) |
| Innate Pharma | IPH-31XX | TLR3 agonist | cancer |
| | IPH-32XX | TLR7 agonist | |
| Inimex | LL-37 (Cathelicidin) | TLR modulator | |
| | | peptide | |
| Juvaris | Cationic lipid - | TLR9 agonist | vaccine |
| BioTherapeutics | DNA complexe | | |
| MultiCell | MCT-465 dsRNA | TLR3 agonist | adjuvant to viral or oncology |
| Technologies | | | vaccines |
| Takeda | TAK-242 | TLR4 antagonist | sepsis |
| | (Resatorvid) | | |
| Vaxinate | Flagellin HuHa | TLR5 agonist linked | vaccine |
| | | to influenza | |
| | | haemagglutinin | |
| | Flagellin HuM2e | linked to M2 | vaccine |
| | | ectodomain of | |
| | | influenza A | |
| 3M | Aldara (Imiquimod) | topical TLR7 | genital warts, HPV, actinic |
| Pharmaceuticals | | agonist | keratoses, superficial basal |
| | | | cell carcinoma |
| | Resiquimod | TLR7 and TLR8 | HepC virus infection |
| | | agonist | |
| Oncovir | Poly-ICLC | TLR3 agonist | Brain cancer |
| Pfizer | 852A | TLR7 agonist | melanoma |
| | CPG-52364 | TLR7, 8, 9 | Systemic lupus |
| | | antagonist | erythematosus (SEL) |
| Dainippon/Sumitomo | DSP-3025 | TLR7 agonist | asthma |
| Pharma | | | |
| Array BioPharma | VTX-463 | TLR7 and TLR8 | allergy |
| | | agonist | |
| ViroGenomics | NPI-503 | TLR4, 7, 8, 9 | Cerebral ischemia |
| | | agonist | |
| Pulmotect | PUL-42 | TLR2/6 and TLR9 | Infection |
| | | agonist | |

Additional TLR agonists or antagonists include lipopeptides, glycerophosphatidylinositol (TLR 1, 2, 6), LPS (TLR 4), microbial nucleic acid dsRNA (TLR 3), microbial nucleic acid ssRNA (TLR 7, 8), microbial protein, e.g. Flagellin (TLR 5), Profilin (TLR 11), Hepatitis B Virus antigen (rHBsAg, Engerix B), Human Papilloma Virus antigen, Fendrix, Supervax, cervarix, Stimuvax/BLP25, Monophosphoryl Lipid A (MPLA), RC-529, 1018 ISS:Class B CpG Oligodeoxynucleotide (ODN), CYT004-MelQbG10, Remune, Pollinex Quattro, AZD1419, Imiquimod. Imidazoquinoline, Imidazoquinoline (Aldara), CpG ODN (DV1079), CpG 52364, as described for example in Amani Makkouk and Alexander M. Abdelnoor, Immunopharmacology and Immunotoxicology, 2009; 31(3): 331-338.

For example, activation of TLR7 and TLR9 present in certain dendritic cells and lymphocytes may be useful for the treatment of various types of cancer by stimulating immunity. In contrast, inhibition of specific TLRs may be useful in treating autoimmune disorders, such as psoriasis and lupus, by blocking the production of multiple pro-inflammatory mediators. In autoimmune diseases, antagonists of TLRs 7, 8, and 9 offer potential treatment for psoriasis and systemic lupus erythematosus (SLE). SLE is an autoimmune disorder in which it is thought that an immune complex of autoantibodies and protein-bound DNA interacts with dendritic cells and subsequently leads to the activation of intracellular TLR9. For cancer treatment, e.g. non-small-cell lung cancer, CpG oligodeoxynucleotides (ODN), which mimics the natural ligand of TLR9-unmethylated bacterial CpG DNA, are administered in combination with anti-cancer agents, such as carboplatin and paclitaxel. CpG oligonucleotides are also being tested for breast and renal cancers, asthma, allergies, hepatitis-B virus and hepatitis-C virus infection. Further, TLR9 agonists are used as (cancer) vaccine adjuvants. Activation of the TLR3 pathway by double-stranded RNA leads to the activation of NFkappaB and the production of type I interferons, which is employed to destroy cancerous cells present in melanoma and breast cancer. A mismatched, double-stranded RNA which activates TLR3 has been developed for the treatment of chronic fatigue syndrome. TLR7/8 agonists are under development for the treatment of allergy. It is thought that shifting the immune response balance in favor of the Th1 response is likely to alleviate the symptoms of allergic hypersensitivity. TLR4 antagonists are being developed for the treatment of sepsis and septic shock. TLR2, 6 and TLR9 agonists are being developed for the treatment of pneumonia and influenza infection.

Methods for modulating an immune response are provided, the methods comprise administering to a subject in need of such treatment i) a formulation comprising a monovalent metal cation or salt thereof, a divalent metal cation or salt thereof or a combination thereof, in an amount sufficient to modulate TLR signaling, and ii) one or more additional TLR agonists or antagonists in an amount sufficient to modulate TLR signaling. Optionally, one or more additional therapeutic agents, such as anti-allergy agents, anti-cancer agents, anti-pathogenic agents, etc. may be administered to the subject.

Further provided herein are methods for improving selectivity of TLR agonists, the method comprising administering i) a broad-specific TLR agonist and ii) a cationic formulation comprising a monovalent metal cation or salt thereof, a divalent metal cation or salt thereof or a combination thereof, in an amount sufficient to modulate TLR signaling, wherein the cationic formulation modifies TLR signaling of the broad-specific TLR agonist so that the resulting TLR signaling is enhanced through TLR1/2, TLR2/6, TLR7, and/or TLR9, and TLR signaling through TLR2, TLR3, TLR4 and/or TLR5 is reduced. Thus, if e.g. TLR2/6 and 9 or TLR7, TLR8 and/or TLR9 activation is desired, but the TLR modifying agents also activates additional TLRs, co-administration of monovalent metal cation or salt thereof, a divalent metal cation or salt thereof or a combination thereof may enhance the desired signaling pathways while reducing the undesired signaling pathways, e.g. through TLR2, TLR3, TLR4 and/or TLR5. If it is desired to reduce signaling through TLR4, e.g. using a TLR4 antagonist, co-administration of a monovalent metal cation or salt thereof, a divalent metal cation or salt thereof or a combination thereof may enhance the desired reduction in TLR4 signaling. If a broad downregulation of TLR signaling is desired, co-administration of monovalent metal cation or salt thereof, a divalent metal cation or salt thereof or a combination thereof, with a TLR7, TLR8, and TLR9 antagonist may be suitable. Various additional useful combinations will be apparent to one of ordinary skill, allowing one to trigger a desired immune response, e.g. Th1-biased, Th2-biased, or unbiased.

It should be appreciated that the formulation comprising a monovalent metal cation or salt thereof, a divalent metal cation or salt thereof or a combination thereof, in an amount sufficient to modulate TLR signaling, and the one or more additional TLR agonists or antagonists may be administered simultaneously, either as separate formulations or as a co-formulation, or may be administered at different times.

TLR signaling may be modified in any cell, although phagocytes are preferred. Phagocytes include, but are not limited to, macrophages, monocytes, granulocytes, neutrophils, basophils, eosinophils and dendritic cells. It should be appreciated that non-phagocytes, e.g. B lymphocytes and epithelial cells also express Toll-like receptors and can be modulated by the methods described herein. For example, TLR1 is expressed by monocytes/macrophages, a subset of dendritic cells, and B lymphocytes; TLR2 is expressed by monocytes/macrophages, myeloid dendritic cells and mast cells; TLR3 is expressed by dendritic cells, and B lymphocytes; TLR4 is expressed by monocytes/macrophages, myeloid dendritic cells, mast cells, B lymphocytes, and intestinal epithelium; TLR5 is expressed by monocytes/macrophages, a subset of dendritic cells, and intestinal epithelium; TLR6 is expressed by monocytes/macrophages, mast cells, and B lymphocytes; TLR7 and 9 are expressed by monocytes/macrophages, plasmacytoid dendritic cells, and B lymphocytes; TLR8 is expressed by monocytes/macrophages, a subset of dendritic cells, and mast cells.

Compositions for modulating an immune response are provided, comprising mono- or divalent metal cation or salts thereof and one or more additional TLR agonists or antagonists, optionally further comprising one or more additional therapeutic agents, such as anti-allergy agents, anti-cancer agents, anti-pathogenic agents, etc. If desired, the compositions described herein can include a physiologically or pharmaceutically acceptable carrier, surfactants or excipient. Examples of pharmaceutically acceptable excipients include, but are not limited to, carbohydrates, amino acids, polyamino acids, metal ions, lipids, surfactants, buffers, salts, polymers, and the like, and combinations thereof, an acidic component, antioxidant, and/or tonicity modifier.

The compositions can be administered by any suitable route, such as orally, parenterally (e.g., intravenous, intra-arterial, intramuscular, or subcutaneous injection), topically, by inhalation (e.g., intra-bronchial, intranasal or oral inhalation, intranasal drops), rectally, vaginally, and the like.

The composition can be a liquid formulation or a gel, foam, etc. comprising one or more solubilized mono- and/or divalent metal ion salts (e.g. NaCl, KCl, CaCl₂, MgCl₂). Alternatively, the composition can be a dry powder comprising one or more soluble mono- and/or divalent metal ion salts (e.g. NaCl, KCl, CaCl₂, MgCl₂). Dry powders may be particularly suitable for reaching the airways, e.g. if it is desired to modulate the immune response in respiratory diseases, such as e.g. asthma, COPD and cystic fibrosis, or in infections, such as infectious pneumonia.

Suitable metal cations preferably are selected from the group consisting of Na⁺, Li⁺, K⁺, Ca²⁺, and Mg²⁺. A salt suitable for the formulations, e.g. liquid or dry powder, can be a monovalent metal cation salt, such as, for example, a sodium salt, potassium salt or a lithium salt.

Suitable sodium salts that can be present in the dry particles include, for example, sodium chloride, sodium citrate, sodium sulfate, sodium lactate, sodium acetate, sodium bicarbonate, sodium carbonate, sodium stearate, sodium ascorbate, sodium benzoate, sodium biphosphate, dibasic sodium phosphate, sodium phosphate, sodium bisulfite, sodium borate, sodium gluconate, sodium metasilicate, sodium propionate and the like.

Suitable potassium salts include, for example, potassium chloride, potassium citrate, potassium bromide, potassium iodide, potassium bicarbonate, potassium nitrite, potassium persulfate, potassium sulfite, potassium sulfate, potassium bisulfite, potassium phosphate, potassium acetate, potassium citrate, potassium glutamate, dipotassium guanylate, potassium gluconate, potassium malate, potassium ascorbate, potassium sorbate, potassium succinate, potassium sodium tartrate and any combination thereof.

Suitable lithium salts include, for example, lithium chloride, lithium bromide, lithium carbonate, lithium nitrate, lithium sulfate, lithium acetate, lithium lactate, lithium citrate, lithium aspartate, lithium gluconate, lithium malate, lithium ascorbate, lithium orotate, lithium succinate or any combination thereof.

A salt suitable for the formulations, e.g. liquid or dry powder, can be a divalent metal cation salt, such as, for example, a calcium salt or a magnesium salt.

Suitable calcium salts that can be present in the dry particles described herein include, for example, calcium chloride, calcium sulfate, calcium lactate, calcium citrate, calcium carbonate, calcium acetate, calcium phosphate, calcium alginate, calcium stearate, calcium sorbate, calcium gluconate and the like.

Suitable magnesium salts that can be present in the dry particles described herein include, for example, magnesium fluoride, magnesium chloride, magnesium bromide, magnesium iodide, magnesium lactate, magnesium phosphate, magnesium sulfate, magnesium sulfite, magnesium carbonate, magnesium oxide, magnesium nitrate, magnesium borate, magnesium acetate, magnesium citrate, magnesium gluconate, magnesium maleate, magnesium succinate, magnesium malate, magnesium taurate, magnesium orotate, magnesium glycinate, magnesium naphthenate, magnesium acetylacetonate, magnesium formate, magnesium hydroxide, magnesium stearate, magnesium hexafluorsilicate, magnesium salicylate or any combination thereof.

Preferred sodium salts are sodium citrate, sodium chloride, sodium lactate, and sodium sulfate. Preferred potassium salts are potassium citrate and potassium sulfate. Preferred calcium salts are calcium lactate, calcium sulfate, calcium citrate, and calcium carbonate. Preferred magnesium salts are magnesium sulfate, magnesium lactate, magnesium chloride, magnesium citrate, and magnesium carbonate.

If desired, the formulations, e.g. liquid or dry powder may further comprise a salt other than a monovalent or divalent metal cation salt. For example, the formulation may comprise a trivalent or other multivalent salt, such as one or more non-toxic salts of the elements aluminum, silicon, scandium, titanium, vanadium, chromium, cobalt, nickel, copper, manganese, zinc, tin, silver and the like.

If desired, to further modulate TLR signaling, the compositions described herein may further comprise one or more agonists or antagonists of TRP channel signaling, for example, Allyl isothiocyanate (AITC), Benyzl isothiocyanate (BITC), Phenyl isothiocyanate, Isopropyl isothiocyanate, methyl isothiocyanate, diallyl disulfide, acrolein (2-propenal), disulfiram (Antabuse®), farnesyl thiosalicylic acid (FTS), farnesyl thioacetic acid (FTA), chlodantoin (Sporostacin®, topical fungicidal), (15-d-PGJ2), 5,8,11,14 eicosatetraynoic acid (ETYA), dibenzoazepine, mefenamic acid, fluribiprofen, keoprofen, diclofenac, indomethacin. SC alkyne (SCA), pentenal, mustard oil alkyne (MOA), iodoacetamine, iodoacetamide alkyne, (2-aminoethyl) methanethiosulphonate (MTSEA), 4-hydroxy-2-noneal (HNE), 4-hydroxy xexenal (HHE), 2-chlorobenzalmalononitrile, N-chloro tosylamide (chloramine-T), formaldehyde, isoflurane, isovelleral, hydrogen peroxide, URB597, thiosulfinate, Allicin (a specific thiosulfinate), flufenamic acid, niflumic acid, carvacrol, eugenol, menthol, gingerol, icilin, methyl salicylate, arachidonic acid, cinnemaldehyde, super sinnemaldehyde, tetrahydrocannabinol (THC or (delta-9) A9-THC), cannabidiol (CBD), cannabichromene (CBC), cannabigerol (CBG), THC acid (THC-A), CBD acid (CBD-A), Compound 1 (AMG5445), 4-methyl-N-[2,2,2-trichloro-1-(4-chlorophenylsulfanyl)ethyl]benzamide, N-[2,2,2-trichloro-1-(4-chlorophenylsulfanyl)ethyl]acetamid, AMG9090, AMG5445, 1-oleoyl-2-acetyl-sn-glycerol (OAG), carbachol, diacylglycerol (DAG), 1,2-Didecanoylglycerol, flufenamate/flufenamic acid, niflumate/niflumic acid, hyperforin, 2-aminoethoxydiphenyl borate (2-APB), diphenylborinic anhydride (DPBA), delta-9-tetrahydrocannabinol ((delta-9) A9-THC or THC), cannabiniol (CBN), 2-APB, 0-1821, 11-hydroxy-(delta 9) A9-tetrahydrocannabinol, nabilone, CP55940, HU-210, HU-211/dexanabinol, HU-331, HU-308, JWH-015, WIN55,212-2,2-Arachidonoylglycerol (2-AG), Arvil, PEA, AM404, O-1918, JWH-133, incensole, incensole acetate, menthol, eugenol, dihydrocarveol, carveol, thymol, vanillin, ethyl vanillin, cinnemaldehyde, 2 aminoethoxydiphenyl borate (2-APB), diphenylamine (DPA), diphenylborinic anhydride (DPBA), camphor, (+)-bomrneol, (−)-isopinocampheol, (−)-fenchone, (−)-trans-pinocarveol, isobomeol, (+)-camphorquinone, (−)-alpha-thujone, alpha-pinene oxide, 1,8-cincole/eucalyptol, 6-tert-butyl-m-cresol, carvacrol, p-sylenol, kreosol, propofol, p-cymene, (−)-isoppulegol, (−)-carvone, (+)-dihydrocarvone, (−)-menthone, (+)-linalool, geraniol, 1-isopropyl-4-methyl-bicyclo[3.1.0]hexan-4-ol, 4 alpha PDD, GSK1016790A, 5′6′Epoxyeicosatrienoic (5′6′-EET), 8′9′Epoxyeicosatrienoic (8′9′-EET), APP44-1, RN1747, Formulation Ib WO 2006/02909, Formulation Ib WO 2006/02909, Formulation Ic WO 2006/02929, Formulation Id WO 2006/02929, Formulation IIb WO 2006/02929, Formulation IIc WO 2006/02929, arachidonic acid (AA), 12-O-Tetradecanoylphorbol-13-acetate (TPA)/phorbol 12-myristate 13-acetate (PMA), bisandrographalide (BAA), incensole, incensole acetate, Compound IX WO 2010/015965, Compound X WO 2010/015965, Compound XI WO 2010/015965, Compound XI WO 2010/015965, WO 2009/004071, WO 2006/038070, WO 2008/065666, Formula VI WO 2010/015965, Formula IV WO 2010/015965, dibenzoazepine, dibenzooxazepine, Formula I WO 2009/071631, N-{(1S)-1-[({(4R)-1-[(4-chlorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(4-fluorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(2-cyanophenyl)sulfonyl]-3-oxohexahydro-1H-azepin-4-yl}amino)carbonyl]-3-methylbutyl}-1-methyl-1H-indole-2-carboxamide, and N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide.

Alternative or in addition, the immune response can further be modulated by one or more agents selected from the group consisting of 2-[(aminocarbonyl)amino]-5-[4-fluorophenyl]-3-thiophenecarboxamide (TPCA-1); doxycycline; NR58-3.14.3; spiropiperidine; N-(6-chloro-9H-beta-carbolin-8-yl) nicotinamide (PS-1145); N-(6-chloro-7-methoxy-9H-beta-carbolin-8-yl)-2-methyl-nicotinamide (ML 120B); N-acetylcysteine (NAC); antagonist anti-CCR2 (CCR2-05) monoclonal antibody; gamma-tocopherol; 2-cyano-3,12-dioxoolean-1,9-dien-28-oic acid (CDDO); 15-deoxy-(delta)Δ(12,14)-prostaglandin J(2) (15d-PGJ(2)); GRP blocking agent 77427; GRP blocking antibody 2A11; IKK2 inhibitor (IMD-0354); GSK-3 inhibitor 3-(2,4-dichlorophenyl)-4-(1-methyl-1H-indol-3-yl)-1H-pyrrole-2,5-dione (SB216763); dehydroevodiamine; evodiamine; rutaecarpine; 5alpha-reductase inhibitor finasteride; cordycepin; Nox2 inhibitors; fluoxetine; chymase inhibitor 2-[4-(5-fluoro-3-methylbenzo[b]thiophen-2-yl)sulfonamido-3-methanesulfonylphenyl]thiazole-4-carboxylic acid (TY-51469); TNF-alpha converting enzyme (TACE) and matrix metalloproteinases (MMPs) dual inhibitors: PKF242-484, PKF241-466; CXCR4 antagonist AMD3100; inhibitor of p44/42 MAPK U0126; IKK-selective inhibitors: PS-1145 [N-(6-chloro-9H-beta-carbolin-8-ly) nicotinamide], ML120B [N-(6-chloro-7-methoxy-9H-beta-carbolin-8-yl)-2-methyl-nicotinamide]; artemisinin; proteasome inhibitors: pyrrolidine dithiocarbamate [PDTC], MG132, PS-341 (bortezomib); bindarit, thromboxane A(2) synthase inhibitor ozagrel; aminopeptidase N inhibitor actinonin; NF-kappa B inhibitor IKK-NBD; p38 MAP kinase inhibitors: SB 203580, SB 202190; neutrophil elastase inhibitor Sivelestat; quercetin (3,3′,4′,5,7-pentahydroxyflavone); N,N-dimethylsphingosine; phosphodiesterase inhibitor pentoxifylline; PKA inhibitor H-89; anti-CCR2-blocking monoclonal antibody MC21; IkappaB-alpha phosphorylation inhibitor BAY 11-7082; alpha-1-antitrypsin; and synthetic metalloprotease inhibitor (RS 113456).

### DEFINITIONS

The term “dry powder” as used herein refers to a composition that contains respirable dry particles that are capable of being dispersed in an inhalation device and subsequently inhaled by a subject. Such a dry powder may contain up to about 25%, up to about 20%, or up to about 15% water or other solvent, or be substantially free of water or other solvent, or be anhydrous.

The term “dry particles” as used herein refers to respirable particles that may contain up to about 25%, up to about 20%, or up to about 15% water or other solvent, or be substantially free of water or other solvent, or be anhydrous.

The term “respirable” as used herein refers to dry particles or dry powders that are suitable for delivery to the respiratory tract (e.g., pulmonary delivery) in a subject by inhalation. Respirable dry powders or dry particles have a mass median aerodynamic diameter (MMAD) of less than about 10 microns, preferably about 5 microns or less.

The term “small” as used herein to describe respirable dry particles refers to particles that have a volume median geometric diameter (VMGD) of about 10 microns or less, preferably about 5 microns or less. VMGD may also be called the volume median diameter (VMD), ×50, or Dv50.

As used herein, the term “respiratory tract” includes the upper respiratory tract (e.g., nasal passages, nasal cavity, throat, and pharynx), respiratory airways (e.g., larynx, trachea, bronchi, and bronchioles) and lungs (e.g., respiratory bronchioles, alveolar ducts, alveolar sacs, and alveoli).

The term “dispersible” is a term of art that describes the characteristic of a dry powder or dry particles to be dispelled into a respirable aerosol. Dispersibility of a dry powder or dry particles is expressed herein as the quotient of the volume median geometric diameter (VMGD) measured at a dispersion (i.e., regulator) pressure of 1 bar divided by the VMGD measured at a dispersion (i.e., regulator) pressure of 4 bar, VMGD at 0.5 bar divided by the VMGD at 4 bar as measured by HELOS/RODOS, VMGD at 0.2 bar divided by the VMGD at 2 bar as measured by HELOS/RODOS, or VMGD at 0.2 bar divided by the VMGD at 4 bar as measured by HELOS/RODOS. These quotients are referred to herein as “1 bar/4 bar,” “0.5 bar/4 bar,” “0.2 bar/2 bar,” and “0.2 bar/4 bar,” respectively, and dispersibility correlates with a low quotient. For example, 1 bar/4 bar refers to the VMGD of respirable dry particles or powders emitted from the orifice of a RODOS dry powder disperser (or equivalent technique) at about 1 bar, as measured by a HELOS or other laser diffraction system, divided by the VMGD of the same respirable dry particles or powders measured at 4 bar by HELOS/RODOS. Thus, a highly dispersible dry powder or dry particles will have a 1 bar/4 bar or 0.5 bar/4 bar ratio that is close to 1.0. Highly dispersible powders have a low tendency to agglomerate, aggregate or clump together and/or, if agglomerated, aggregated or clumped together, are easily dispersed or de-agglomerated as they emit from an inhaler and are breathed in by a subject. Dispersibility can also be assessed by measuring the size emitted from an inhaler as a function of flow rate. VMGD may also be called the volume median diameter (VMD), ×50, or Dv50.

As used herein, the term “emitted dose” or “ED” refers to an indication of the delivery of a drug formulation from a suitable inhaler device after a firing or dispersion event. More specifically, for dry powder formulations, the ED is a measure of the percentage of powder that is drawn out of a unit dose package and that exits the mouthpiece of an inhaler device. The ED is defined as the ratio of the dose delivered by an inhaler device to the nominal dose (the mass of powder per unit dose placed into a suitable inhaler device prior to firing). The ED is an experimentally-measured parameter, and can be determined using the method of USP Section 601 Aerosols, Metered-Dose Inhalers and Dry Powder Inhalers, Delivered-Dose Uniformity, Sampling the Delivered Dose from Dry Powder Inhalers, United States Pharmacopeia convention, Rockville, Md., 13^{th} Revision, 222-225, 2007. This method utilizes an in vitro device set up to mimic patient dosing.

The term “effective amount,” as used herein, refers to the amount of a therapeutic agent needed to achieve the desired therapeutic or prophylactic effect, such as an amount that is sufficient to reduce pathogen (e.g., bacteria, virus) burden, reduce symptoms (e.g., fever, coughing, sneezing, nasal discharge, diarrhea and the like), reduce occurrence of infection, reduce viral replication, or improve or prevent deterioration of respiratory function (e.g., improve forced expiratory volume in 1 second FEV₁ and/or forced expiratory volume in 1 second FEV₁ as a proportion of forced vital capacity FEV₁/FVC, reduce bronchoconstriction), produce an effective serum concentration of a therapeutic agent, increase mucociliary clearance, reduce total inflammatory cell count, or modulate the profile of inflammatory cell counts. The actual effective amount of a therapeutic agent for a particular use can vary according to the particular therapeutic agent(s), the mode of administration, and the age, weight, general health of the subject, the condition or disease treated, and the severity of the symptoms or condition being treated.

The term “pharmaceutically acceptable excipient” as used herein means that the excipient can be taken into the lungs with no significant adverse toxicological effects on the lungs. Such excipients are generally regarded as safe (GRAS) by the U.S. Food and Drug Administration.

A “biomarker” as used herein, refers to a polypeptide (e.g. a protein) or an oligonucleotide (e.g. a nucleic acid) that can be detected and measured in body fluids, or in samples obtained therefrom, whose presence/absence or concentration may be correlated to the presence or absence of an inflammation, irritation, and/or infection in a subject. Biomarkers might be detected in a subject using e.g. genomics, proteomics or imaging technologies. A biomarker may include any of, but is not limited to, a cytokine, chemokine, growth factor, enzyme or other protein associated with an inflammation, irritation, and/or infection in the subject. A biomarker can also include a nucleic acid that encodes any of the above proteins or an mRNA or microRNA that is differentially expressed in a subject having an inflammation, irritation, and/or infection.

All references to salts (e.g., calcium salts) herein include anhydrous forms and all hydrated forms of the salt.

All weight percentages for dry powders are given on a dry basis.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLES

### Example 1

### Effective Doses of Calcium Ions Determined in Pre-Clinical Models

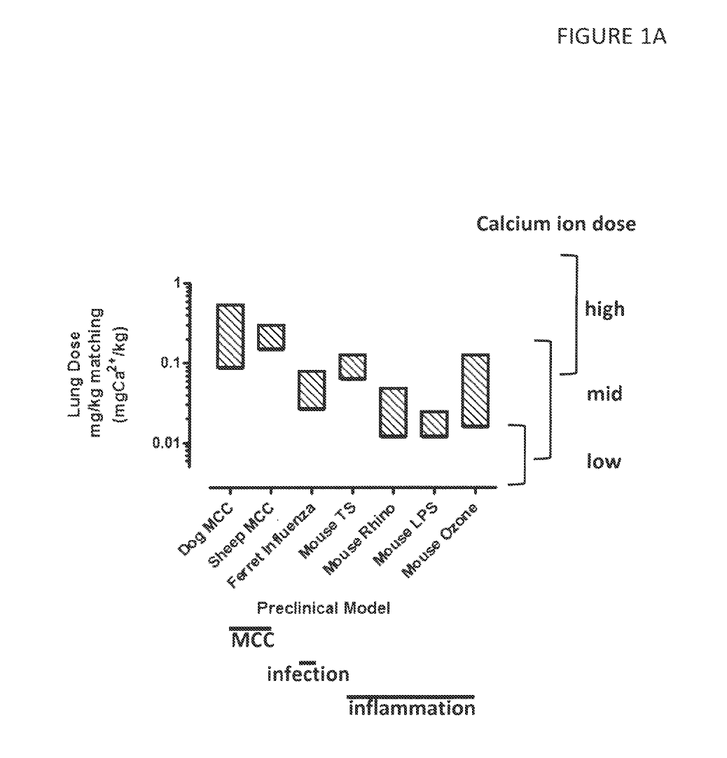

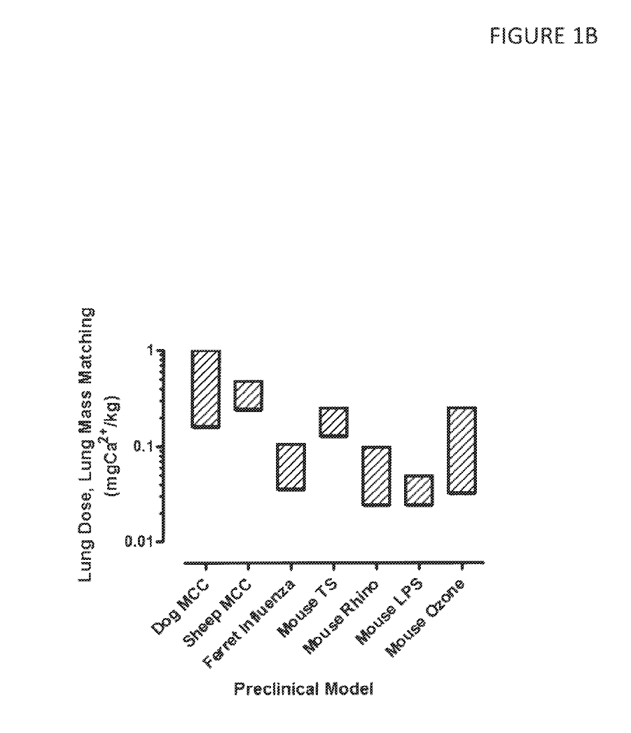

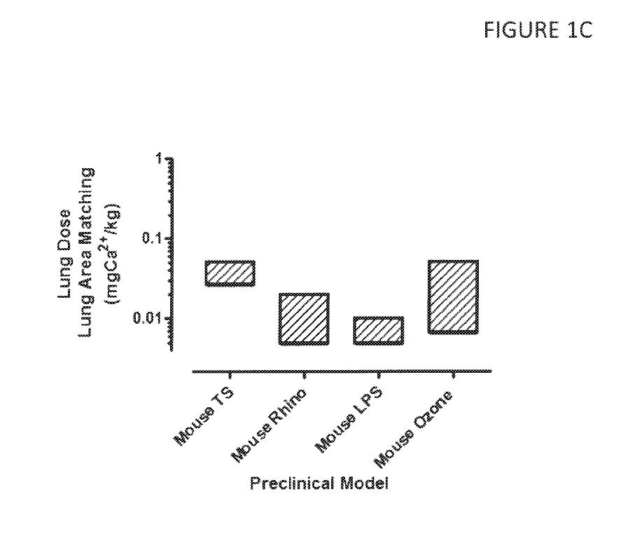

Calcium lung doses were calculated from various animal models of infection, inflammation and mucociliary clearance (MCC), shown in FIG. 1A-C. The animal models used are described in detail in PCT Publication Nos. WO 2012/030664 “DRY POWDER FORMULATIONS AND METHODS FOR TREATING PULMONARY DISEASES” and WO 2010/111680 “DRY POWDER FORMULATIONS AND METHODS FOR TREATING PULMONARY DISEASES”.

Briefly, in all preclinical models, aerosol concentrations and size distributions were measured at the point of aerosol exposure, which was typically by nasal inhalation. Exposure durations were controlled and minute volumes calculated for the animal based on empirical correlations such as e.g. that of Bide et al. J. App. Toxicol. 20:273-90 (2000) to determine the aerosol exposed dose. Empirically validated lung deposition parameters were incorporated for each species to predict lung deposition based on aerosol exposed dose.

In the ferret model, dry powder aerosols were delivered by nose-only inhalation as described in PCT Publication No. WO 2012/030664 “DRY POWDER FORMULATIONS AND METHODS FOR TREATING PULMONARY DISEASES”. To determine the lung deposition fraction, a small amount of DTPA was spray dried into the efficacious calcium formulation (10.0% leucine, 58.3% calcium lactate, 31.2% sodium chloride, 0.5% diethylene triamine pentaacetic acid (DTPA)), to allow the powder to be radio-labeled with ^{99m}Tc. The radiolabel was validated by comparing the size distribution by radioactivity to the size distributions by mass before and after radio-labeling. Immediately following dose administration, the regional deposition of the radio-labeled aerosol was measured by combined 3D SPECT/CT imaging. SPECT/CT combines high resolution anatomical 3D computerized tomography (CT) and single photon emission computerized tomography (SPECT) as functional imaging. From these measurements, a predicted lung dose of 8.8% of the total aerosol exposed dose was determined and subsequently used to calculate the lung dose in ferret efficacy studies in conjunction with measured aerosol exposed doses in the studies.

In sheep and dog models of mucociliary clearance, aerosol exposures were performed via a tracheal tube with a Harvard pump controlling the breathing pattern of the animals during the dosing period as described in PCT Publication No. WO 2012/030664 “DRY POWDER FORMULATIONS AND METHODS FOR TREATING PULMONARY DISEASES”. Gamma scintigraphy studies with a model aerosol found that the lung deposition fraction of the aerosol dose exiting the tracheal tube was 30%.

In multiple mouse models, mice were exposed by whole body exposure to dry powder aerosols delivered by capsule based DPIs as described in PCT Publication No. WO 2012/030664 “DRY POWDER FORMULATIONS AND METHODS FOR TREATING PULMONARY DISEASES”. Bovine IgG was spray dried into a dry powder calcium formulation at 1.3% (w/w) as a tracer agent. After aerosol exposures at multiple dose levels, bovine IgG was recovered from the mice lungs. The lung deposition fraction of the total aerosol exposed dose was 4.8% and was used in subsequent efficacy studies in conjunction with measured aerosol exposed doses in the studies.

The efficacious lung doses in the preclinical models were translated to human equivalent lung doses by one of three methods illustrated in FIG. 1A-C. In FIG. 1A, the lung dose in mg Ca²⁺ ion/kg bodyweight in the model animal was calculated and the equivalent human lung dose has the same value in mg Ca²⁺ ion/kg bodyweight of the person. In FIG. 1B, for each preclinical model, the mass of Ca²⁺ ion deposited in the lung of the model animal was scaled by the ratio of the lung masses of the two species and then reported as a human equivalent dose in mg Ca²⁺ ion/kg bodyweight of the person. In FIG. 1C, for each preclinical model, the mass of Ca²⁺ ion deposited in the lung of the model animal was scaled by the ratio of the lung surface areas of the two species and then reported as a human equivalent dose in mg Ca²⁺ ion/kg bodyweight of the person. Exemplary dose calculations for one calcium salt formulation, Formulation II, are summarized in Table 2.

TABLE 2
Doses of Formulation II in human subjects.

| Nominal | Nominal | | Predicted Human |
|---|---|---|---|
| Human | Powder | Predicted Human | Lung Dose |
| Dose | Load (mg | Lung Dose | (mg Ca²⁺ ion/kg |
| (mg Ca²⁺ ion) | Formulation II) | (mg Ca²⁺ ion) | bodyweight)* |
| 2.8 | 20 | 1.0 | 0.020 |
| 5.5 | 40 | 2.1 | 0.041 |
| 11 | 80 | 4.1 | 0.082 |
| 22 | 160 | 8.2 | 0.16 |

*calculated for a person with 50 kg bodyweight.

### Example 2

### Calcium-Containing Formulations Increased Airway Surface Lining (ASL) Height

Human Airway Cell Cultures:

The experiments detailed in this Example utilized cultured primary human bronchial epithelial cells (NHBE). These cells were obtained from excess tissue from donor lungs and excised recipient lungs that were obtained at the time of lung transplantation. Cells from the excised bronchial specimens were isolated utilizing protease digestion. Primary isolated cells were seeded (1×10⁶ cells/cm²) on 12-mm permeable support (Transwell-Clear; Costar) pre-coated with human placental collagen. Cells were maintained under air-liquid conditions, washed every 48-72 hours to remove accumulated mucus, and studied as fully differentiated cultures (about 6 weeks, cultures with transepithelial resistances of greater than 200 ohms per square centimeter (Ω/cm²)). All incubations were performed in a well-humidified (about 95%) tissue culture incubator (5% CO₂) at 37° C.

Measurement of ASL Hydration Dynamics:

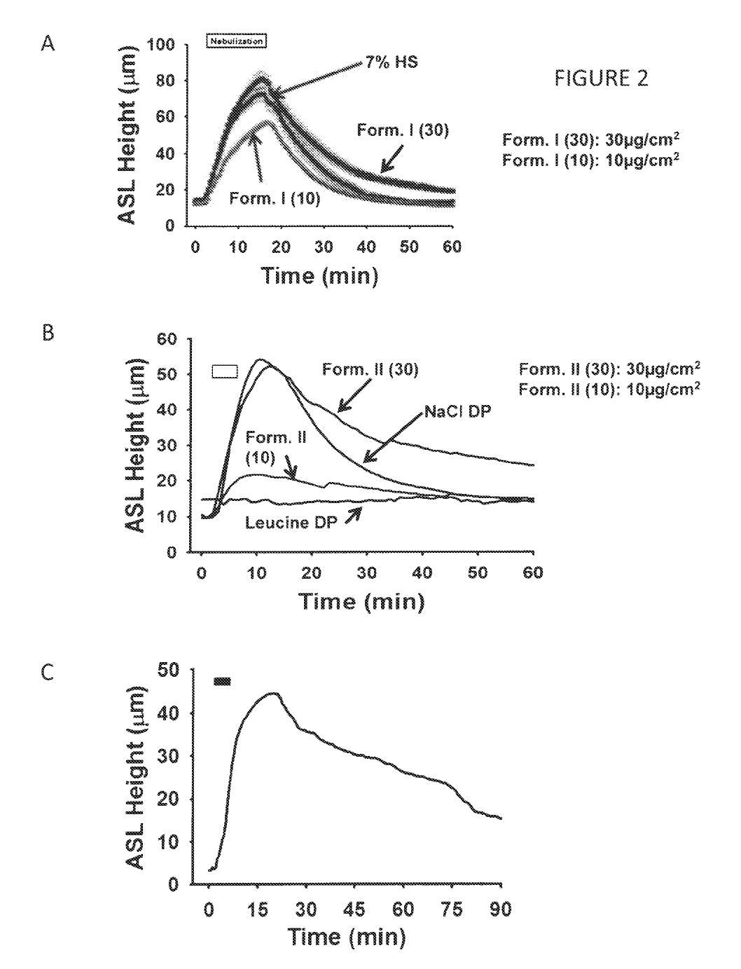

A calcium liquid formulation (which is 9.4% CaCl₂ (w/v), 0.62% NaCl (w/v) in water (Formulation 1), at a concentration resulting in a tonicity factor of 8 times isotonic, FIG. 2A), and a calcium dry powder formulation, Formulation 11 (20% (w/w) leucine, 75% (w/w) calcium lactate, 5% (w/w) sodium chloride, FIG. 2B) were tested for their effect on the level of airway surface layer (ASL) hydration. The effective clearance of particles deposited on airway surfaces during normal breathing requires the coordinated activities of a two-phase system on the airway surface: (i) the periciliary layer (PCL) that extends from the cell surface to the height of the extended cilium; and (ii) the mucus layer that is positioned atop the cilia. The hydration of these (ASL) are normally determined by the net activities of active ion transport systems, where normal airway epithelia have the capacity both to absorb and to secrete salt, with water moving osmotically in response to the generated salt gradients. Agents that increase the level of ASL hydration are predicted to make the mucus more clearable. To understand the magnitude and duration of such an effect, ASL height was measured in real-time following administration of calcium salt formulations.

Freshly washed NHBE cells were pre-stained by a 15 minutes exposure to 10 micromolar calcein-AM to visualize the airway epithelial cells. To visualize the ASL, isotonic saline containing 0.2% vol/vol Texas Red-dextran (70 kDa, Invitrogen) was briefly nebulized onto the lumen of freshly washed airway cultures. This volume of PBS only resulted in minor increase in the ASL height of about 3 microns, for a total pre-study thickness of about 10 microns. Sequential images of the cells and ASL layer were acquired every 30 seconds by laser-scanning confocal microscopy (Model SP5; Leica) using the appropriate filters (540 nm excitation/630 emission and 488 excitation/530 nm emission for Texas Red and calcein, respectively). Images were continuously obtained for up to 45 minutes following formulation delivery. The height of the cell and the ASL layers were calculated from the individual images in an automated process using software based on MatLab.

Normal human bronchial epithelial (NHBE) cells were i) treated over a period of 15 minutes with nebulized calcium salt formulation (FIG. 2A) or ii) treated by instant deposition of a DP calcium salt formulation (FIG. 2B), with NaCl formulated either as a liquid formulation (hypertonic saline (HS)) or as a DP, and leucine formulated either as a liquid formulation or as a DP, respectively, as controls. Changes in ASL height were measured in real-time. Treatment with HS steadily increased ASL height during the dosing period and ASL height gradually returned to baseline after dosing was stopped. A calcium ion formulation (Formulation I) possessed similar tonicity to HS and had a similar effect when dosed for the same time period (deposited about 30 microgram calcium ion per cm²) or about one half the effect when delivered at one third of the dose (deposited about 10 microgram calcium ion per cm²) (FIG. 2A). The effect of dry powder calcium ion formulation (Formulation II) on airway hydration was significantly prolonged when compared to HS or to a dry powder NaCl formulation (FIG. 2B). Data were representative of 3 different donors. Dry powder Formulation II was deposited on the apical surface of CF HBE cells (bronchial epithelial cells derived from a patient with cystic fibrosis) and changes in ASL height were measured in real-time. Formulation II rapidly increased ASL height following deposition and ASL height gradually returned to baseline (FIG. 2C). These data show that calcium-containing formulations are effective in increasing ASL height in CF patient lung cells and may be used to treat patients with cystic fibrosis.

### Example 3

### Calcium-Containing Formulations Enhanced Mucociliary Clearance (MCC) In Vivo

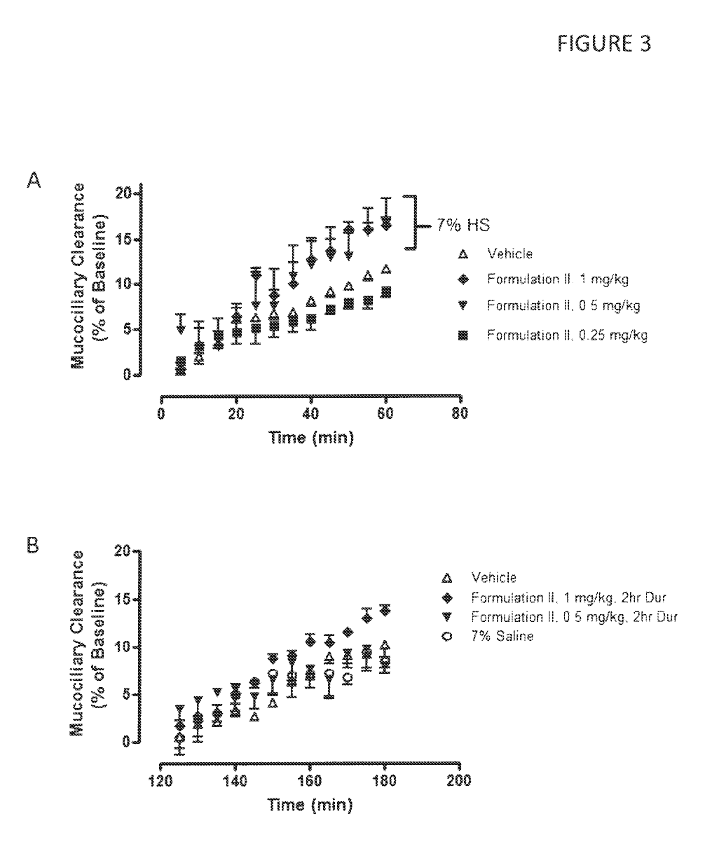

A calcium salt dry powder formulation, Formulation II, of 20% (w/w) leucine, 75% (w/w) calcium lactate, 5% (w/w) sodium chloride was evaluated in an established sheep mucociliary clearance (MCC) model. MCC was evaluated in groups of two to four healthy sheep by measurement of the clearance of pulmonary Tc^{99m}-labeled sulfur colloid aerosols that were delivered by inhalation. The radio-labeled sulfur colloid aerosol was delivered to each of the sheep either immediately following (FIG. 3A) or two hours after (FIG. 3B) the completion of the treatment aerosol exposures and MCC determined via the collection of serial images for an additional 60 minutes. Animals were conscious, supported in a mobile restraint, intubated with a cuffed endotracheal tube and maintained consciousness for the duration of the study.

A rotating brush generator (RBG1000, Palas) was used to generate the dry powder aerosol. The single sheep exposure system was connected to a dosimeter system consisting of a solenoid valve and a source of compressed air (20 psi). The output of the nebulizer is connected to a T-piece, with one end attached to a respirator (Harvard Apparatus Inc., Holliston, Mass.). The system was activated for 1 second at the onset of the inspiratory cycle of the respirator, which was set at an inspiratory/expiratory ratio of 1:1 and a frequency of 20 breaths per minute. A tidal volume of 300 ml was used to deliver the nebulized formulations. Doses of the dry powder were delivered for 15 minutes with the aerosol continuously generated by the RBG at various aerosol concentrations.

After ^{99m}TC-SC nebulization, the animals were immediately extubated and positioned in their natural upright position underneath a gamma camera (Dyna Cam, Picker Corp., Northford, Conn.) so that the field of image was perpendicular to the animals' spinal cord. After acquisition of a baseline image, serial images were obtained at 5 minute intervals for one hour. All images were obtained and stored in the computer for analysis. An area of interest was traced over the image corresponding to the right lung of the animals, and counts were recorded. The left lung was excluded from analysis because its corresponding image was superimposed over the stomach and counts could be affected by swallowed radiolabeled mucus. The counts were corrected for decay and clearance expressed as the percentage reduction of radioactivity present from the baseline image.

The dose delivered for the formulations was measured in vitro with a breathing simulator system drawing the inspiratory flow through filter samples collected at the distal end of a tracheal tube. For the calcium salt dry powder, 1.5 minute filter samples were assayed for deposited calcium by HPLC and the average rate of calcium deposition was determined. From this, the doses delivered in 15 minutes to a 50 kg sheep (exposed doses) were calculated to be 0.25, 0.5 and 1 mg Ca²⁺/kg. These measured doses correspond to the dose delivered from the distal end of the tracheal tube to the sheep during treatment.

The sheep mucociliary clearance model is a well established model with vehicle clearance typically measuring approximately 5-10% at 60 minutes after delivery of the radioactive aerosol (see for example Coote et al, 2009, JEPT 329:769-774). It is known in the art that average clearance measurements greater than about 10% at 60 minutes post baseline indicate enhanced clearance in the model. The time course of clearance when measuring MCC from 2 to 3 hours post dosing is shown in FIG. 3B. The calcium salt formulation at an exposed dose of 1 mg Ca²⁺/kg (n=4) showed enhanced mucociliary clearance with clearance at 60 minutes post baseline (180 minutes post dosing) of 13.8%±1.1% (mean±SE) and surprisingly, exhibited a longer duration of action compared to hypertonic saline. A lower exposed dose of the calcium salt formulation at 0.5 mg Ca²⁺/kg (n=2) and 7% hypertonic saline (n=2) showed mucociliary clearance at 60 minutes post baseline (180 minutes post dosing) of less than 10% and no different than expected baseline clearance (between 5-10%) and measured vehicle control for baseline clearance (open triangles). The data show that calcium salt based dry powder and hypertonic liquid formulations can be used to increase mucociliary clearance.

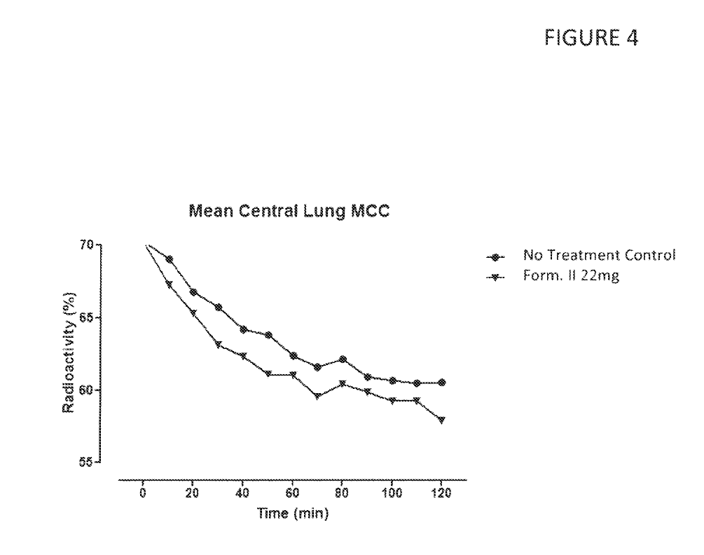

The impact a 22 mg nominal calcium dose (predicted human lung dose of 0.16 mg calcium ion per kg bodyweight) of Formulation II on mucociliary clearance (MCC) velocity in human patients was measured using gamma scintigraphy. The human patients were ex-smoking subjects, aged 45-75, with mild (GOLD stage 0-2), stable COPD. Gold (Global Initiative for Chronic Obstructive Lung Disease) Stage 1 and 2 COPD are defined as reduced FEV/FVC ratio less than 70% predicted and a reduced post-bronchodilator FEV1% predicted of greater than 80% for stage 1, 50-80% for stage 2. ^{99m}Tc-sulphur colloid was administered to the lungs via nebulizer using a standard protocol and serial scintigraphic images acquired for 2 hours. The initial lung deposition pattern and subsequent retention was determined. The data were analyzed to determine clearance parameters for the whole, central and peripheral lung. Initial deposition data confirmed comparable deposition patterns across subjects. The average baseline whole lung clearance rate of 0.3±0.13% per minute over 0-30 minutes was consistent with published data. The data revealed a trend for 22 mg nominal dose (predicted lung dose of 0.16 mg Ca²⁺ ion/kg bodyweight) of Formulation II to increase MCC velocity for both the whole and central lung over the 0 to 2 hour assessment period (FIG. 4). The mean central lung clearance in percent per minute for no treatment (vehicle control) for the 0-120 minute measurement period was 0.117 (±0.041) %/min with n=18 compared to 0.147 (±0.066)%/min with n=17 for Formulation II treatment. The mean central lung clearance in percent per minute for no treatment (control) for the 60-120 minute measurement period was 0.043 (±0.035)%/min compared to 0.073 (±0.066)%/min for Formulation II treatment. An overall 25% increase in MCC velocity for Formulation II versus baseline (vehicle control) over the 120 minutes assessment period was seen. For the time period between 60-120 minutes the increase in MCC velocity for Formulation II versus baseline (vehicle control) was more than 65%. These data confirmed the results obtained from the preclinical models and show that calcium salt formulations may be administered to human patients to increase or augment MCC and may have a longer clearance duration than other osmotic agents such as HS.

### Example 4

### Calcium-Containing Dry Powder Formulations Reduce Expression of Pro-Inflammatory Protein Mediators in COPD Patients

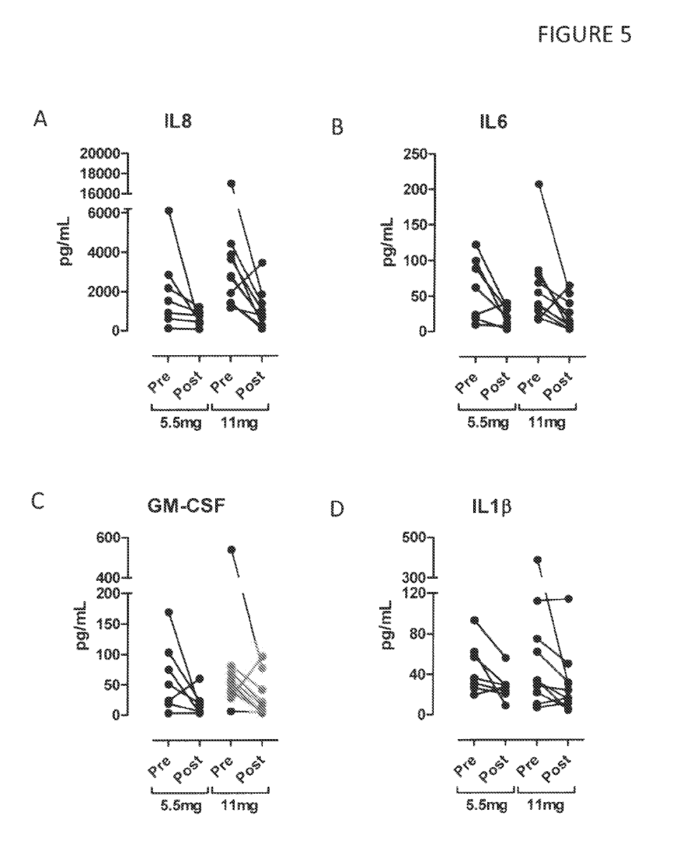

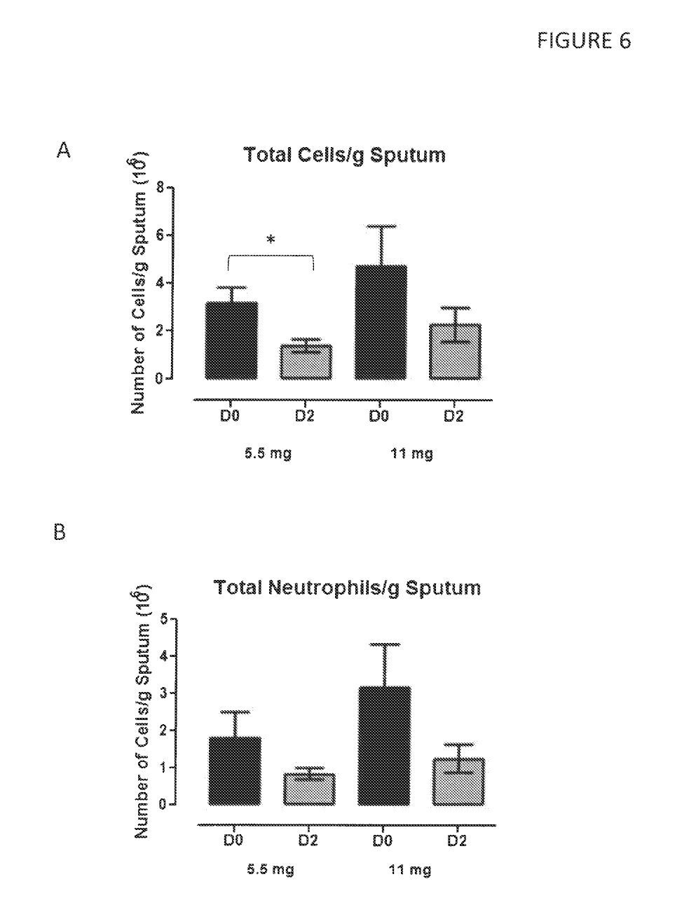

The effect of calcium salt containing dry powders (DP) on inflammatory mediators and inflammatory cell ingress into the airways of mild COPD (GOLD stage 0-2) patients was investigated. 25 subjects were treated with a) a nominal dose of 5.5 mg of calcium ion, equivalent to an emitted dose of 0.087 mg Ca²⁺ ion/kg and equivalent to a fine particle dose of 0.041 mg Ca²⁺ ion/kg (n=12), or a nominal dose of 11 mg of calcium ion, equivalent an emitted dose of 0.17 mg Ca²⁺ ion/kg and equivalent to a fine particle dose of 0.081 mg Ca⁺ ion/kg (n=13) of Formulation II (20% (w/w) leucine, 75% (w/w) calcium lactate, 5% (w/w) sodium chloride) for 3 doses. A short-acting bronchodilator, Salbutamol, was administered prior to each dose. Subjects were stable, with no respiratory infections within 30 days of dosing. Sputa were induced before dosing and 4 hours after the last dose. Sputum levels of the inflammatory mediators, including IL-8, IL-6, GM-CSF, and IL-1 beta, were assessed by immunoassay (FIG. 5A-D), and inflammatory cell counts pre-(D0) and post-(D2) treatment were quantified (FIG. 6A, 6B).

Treatment with Formulation II at both dose levels was well tolerated. FIG. 5 shows mean sputum levels of the four inflammatory mediators IL-8 (FIG. 5A), IL-6 (FIG. 5B) GM-CSF (FIG. 5C), and IL-1 beta (FIG. 5D) declined with treatment for both dose groups. Total inflammatory cells (FIG. 6A) and neutrophils (FIG. 6B) were also reduced upon treatment with Formulation II. These data support the conclusion that inhaled calcium salt formulation treatment reduces airway inflammatory cells and the mediators responsible for their recruitment into the airways of human subjects with COPD.

Prior to the first dose and 2 hours post-third dose sputum and blood (serum/plasma) samples were collected and analyzed for biomarkers of inflammation. Tables 3 and 4 summarize the results for biomarkers before and after Formulation II administration with prior administration of an inhaled short-acting bronchodilator for the 5.5 mg nominal dose (predicted human lung dose of 0.041 mg calcium ion per kg bodyweight) and the 11 mg nominal dose (predicted human lung dose of 0.082 mg calcium ion per kg bodyweight), respectively.

TABLE 3
Sputum and serum/plasma inflammatory biomarkers in COPD
patients for Formulation II treatment with 5.5 mg nominal dose.
5.5 mg nominal calcium ion dose of Formulation II

| | | | |
|---|---|---|---|
| | | Mean Pre- | Mean Post- |
| Analyte | n | dose (±SD) | dose (±SD) |

Blood Analytes

| | | | |
|---|---|---|---|
| Serum CRP (g/L) | 11 | 7 (±6) | 5 (±5) |
| Plasma Fibrinogen (g/L) | 9 | 4 (±1) | 4 (±1) |

Sputum Analytes

| | | | |
|---|---|---|---|
| TNF-α (pg/mL) | 7 | 6 (±5) | 4 (±2) |
| IL-8 (pg/mL) | 7 | 2041 (±2019) | 608 (±383) |
| IL-6 (pg/mL) | 7 | 60 (±44) | 21 (±14) |
| IL-2 (pg/mL) | 7 | 22 (±23) | 15 (±15) |
| IL-1β (pg/mL) | 7 | 47 (±26) | 28 (±14) |
| INF-γ (pg/mL) | 7 | 13 (±11) | 3 (±0) |
| GM-CSF (pg/mL) | 7 | 63 (±58) | 18 (±20) |
| MMP-1 (pg/mL) | 7 | 172 (±67) | 117 (±48) |
| MMP-9 (pg/mL) | 7 | 70830 (±52676) | 55587 (±39662) |
| MPO (ng/mL) | 7 | 15 (±6) | 11 (±4) |
| Neutrophil Elastase (pg/mL) | 6 | 126 (±69) | 100 (±68) |

TABLE 4
Sputum and serum/plasma inflammatory biomarkers in COPD
patients for Formulation II treatment with 11 mg nominal dose.
11 mg nominal calcium ion close of Formulation II

| | | | |
|---|---|---|---|
| | | Mean Pre- | Mean Post- |
| Analyte | n | dose (±SD) | dose (±SD) |

Blood Analytes

| | | | |
|---|---|---|---|
| Serum CRP (g/L) | 11 | 3 (±3) | 3 (±2) |
| Plasma Fibrinogen (g/L) | 10 | 3 (±1) | 3 (±1) |

Serum Analytes

| | | | |
|---|---|---|---|
| TNF-α (pg/mL) | 10 | 36 (±89) | 6 (±6) |
| IL-8 (pg/mL) | 10 | 4036 (±4693) | 1040 (±1010) |
| IL-6 (pg/mL) | 10 | 63 (±56) | 23 (±23) |
| IL-2 (pg/mL) | 10 | 95 (±157) | 11 (±13) |
| IL-1β (pg/mL) | 10 | 77 (±114) | 30 (±32) |
| INF-γ (pg/mL) | 10 | 6 (±6) | 4 (±2) |
| GM-CSF (pg/mL) | 10 | 96 (±157) | 28 (±34) |
| MMP-1 (pg/mL) | 10 | 269(±254) | 191 (±375) |
| MMP-9 (pg/mL) | 10 | 102990 (±82978) | 57127 (±73811) |
| MPO (ng/mL) | 6 | 19 (±11) | 19 (±17) |

The data support the conclusion that administration of Formulation II reduces the levels of pro-inflammatory cytokines, such as, TNF-alpha, IL-8, IL-6, IL-2, IL1-beta, INF-gamma, GM-CSF, MMP-1 and MMP-9, associated with neutrophil infiltration into the airways in subjects with COPD.

### Example 5

### Calcium Salt Formulations Attenuate Allergen-Induced Eosinophilic Bronchitis

Asthma, an allergic airway inflammatory condition, is associated with increased eosinophils in the airways, and in many instances in lung tissue and peripheral blood, which can correlate with asthma severity. Airway eosinophilia has been observed in chronic stable asthma, after allergen inhalation and during exacerbations. When activated by various stimuli, eosinophils release toxic products including oxygen radicals, basic proteins, cytokines and cysteinyl leukotrienes that cause epithelial damage and desquamation in the airway and increased airway hypersensitivity. Bronchial inflammation is considered to be a cause of symptoms and airflow limitation in asthma. Induced sputum is a reliable, noninvasive method to safely obtain airway secretions (Pizzichini Am J Respir Crit Care Med 1996; 154:308-317). Eosinophilic-predominant airway inflammation is typically observed in asthmatic subjects compared to healthy non-asthmatic control subjects. In susceptible individuals, allergic sensitization results after allergens have been taken up and processed by antigen-presenting cells residing in airway epithelium. Both the adaptive and innate immune systems contribute to the recognition and host response to allergens within the respiratory tract. For example, pollen grains (birch and grass) attract and activate neutrophils and eosinophils.

A liquid calcium salt formulation of 1.29% calcium chloride dissolved in 0.9% isotonic saline (Formulation V) was used to test the effect of calcium salt containing formulations on attenuation of eosinophilic bronchitis caused by inhaled aeroallergens in mild atopic asthmatic human subjects. Seven such mild atopic, steroid-naïve asthmatic subjects inhaled Formulation V or matching placebo (isotonic saline) for 3 doses before a whole lung allergen inhalation challenge to an antigen to which the subject was sensitized, as previously identified by skin prick test (e.g. dust mite, grass pollen, ragweed pollen, and cat dander). FEV₁ was monitored for 7 hours and sputum was induced with hypertonic saline at the end of 7 hours. The doses were administered by nebulization of a 5.5 mL ampoule of formulation measured to be equivalent to a dose of 0.32 mg Ca²⁺ ion/kg emitted from the nebulizer with 0.16 mg Ca⁺ ion/kg inhaled dose and 0.097 mg Ca⁺ ion/kg fine particle dose (FPD<5.0 micrometers) as measured in vitro with tidal breathing simulation. The percentage of eosinophils was identified using Wright's stain on a dithiothreitol (DTT)-dispersed sample separated from saliva.

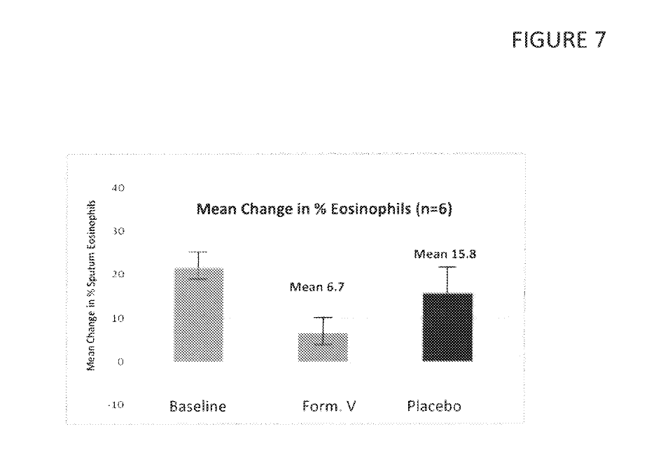

The calcium salt formulation was well tolerated by all subjects. Results were obtained for 6 out of the 7 subjects. The mean change in FEV₁ at 1 hour and 2 hours after calcium salt formulation inhalation were −0.6% and +1.3% respectively compared to −0.9% and +1.0% respectively after placebo inhalation. The percent sputum eosinophilia measured in the induced sputum samples post allergen challenge was significantly less after calcium salt formulation compared to placebo (See FIG. 7).

These data show that the liquid aerosolized calcium salt formulation does not cause bronchoconstriction and attenuates allergen-induced eosinophilic bronchitis. These data support the hypothesis that this may be an effective strategy to protect against bronchitis caused by inhaled particles.

### Example 6

### Barrier Effects of Calcium Formulations in a Mucus Mimetic Model

The apical surface of the airway epithelium is lined with airway lining fluid (ALF) consisting of a mucus gel layer and a periciliary layer. The ALF is rheologically active and serves as a barrier to environmental and infectious particulate to shield the epithelium from external insult. A pass through system was developed to model the interaction between particulate material and mucus and to understand how changes in the rheological properties of mucus affect the movement of bacterial pathogens through mucus. This pass through model is described for example in PCT Publication No. WO 2010/111641 “METHODS FOR TREATING AND PREVENTING PNEUMONIA AND VENTILATOR-ASSOCIATED TRACHEOBRONCHITIS”, see Example 1, pages 54-61. Using this system, it was demonstrated that the topical application of liquid containing calcium salts significantly reduced the movement of bacterial pathogens across mucus mimetic in a dose dependent manner.

Sodium alginate (Sigma Aldrich, St. Louis, Mo.) 4% mucus mimetic (200 microliter) was added to the apical surface of 12 mm Transwell culture inserts (3.0 micron pore size; Costar) and left up to 2 hours at room temperature. Liquid formulations were nebulized into the chamber and allowed to settle by gravity over a 5 minute period. Dry powder formulations were delivered with a DP-4m Penn-Century dry powder insufflator inserted into the sedimentation chamber. After the exposure of the mucus mimetic to the indicated formulations, 10 microliter of bacterial suspension (1:10 dilution of an OD₆₀₀ of about 0.3) was added to the apical surface of the mimetic and 0.5-1.0 mL of sterile isotonic saline was added to the basolateral chamber of the Transwell. At different time points after the addition of bacteria to the apical surface, a sample of the basolateral buffer was collected and the number of bacteria was determined by serial dilution and plating 100 microliter of each dilution on tryptic soy blood agar plates. Plates were incubated overnight at 37° C. and 5% CO₂ and the number of colony forming units on each plate were enumerated (CFU/mL of buffer).

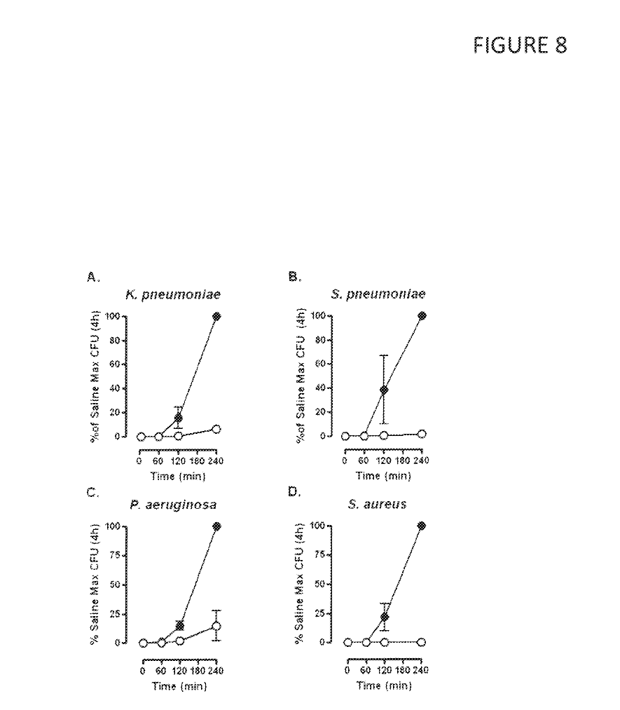

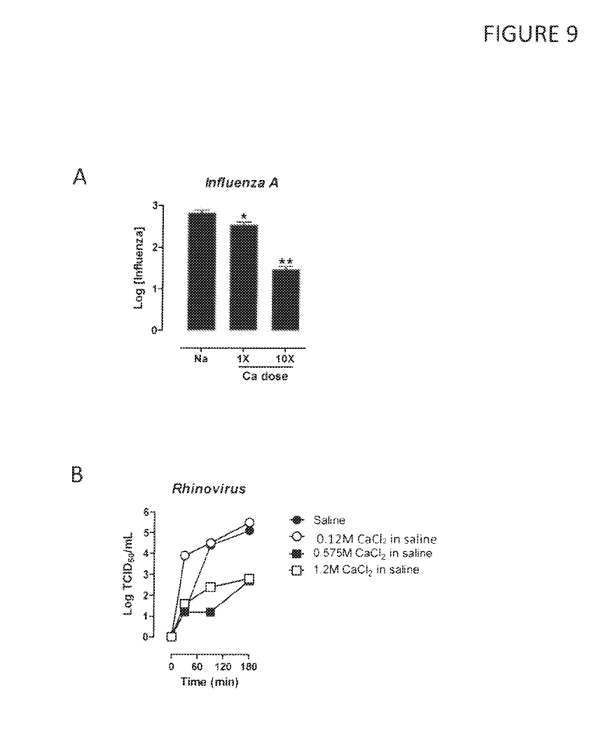

To determine the effect of calcium exposure on bacterial movement in each condition, mimetic was exposed to either 0.12 M CaC₂/0.15 M NaCl (Formulation V) or 1.2 M CaCl₂/0.15 M NaCl (calcium chloride in isotonic saline) using a sedimentation chamber. The approximate dose of calcium delivered to the mimetic using the low dose formulation was 3-5 microgram calcium per cm². This dose was delivered by 5 consecutive bursts of the nebulizers spaced 5 minutes apart. Mimetic was exposed to each formulation and bacteria were added to the apical surface immediately after the last nebulization. In the saline treated control wells, bacteria were first recovered from the basolateral chamber 120 minutes after the addition of bacteria to the apical surface and the titer increased significantly between 120 and 240 minutes (FIG. 8). In contrast, the movement of bacteria through the calcium treated mimetic was delayed and significantly reduced.

K. pneumoniae (Gram-negative; rod shaped, about 0.3-1 micron in diameter), S. pneumoniae (Gram-positive; chains of diplococci, about 0.5-1.25 micron in diameter), P. aeruginosa (Gram-negative; rod shaped, about 0.6-0.8 micron in diameter), and S. aureus (Gram-positive; clumps of cocci, about 0.5-1 micron in diameter) were tested. Exposure of the mimetic to calcium sharply reduced the movement of S. pneumoniae [2.0±2.0% of control at 4 hours (n=3)], P. aeruginosa [14.7±13.0% of control at 4 hours (n=2)], and S. aureus [0.06%±0.007% of control at 4 hours (n=3)], indicating that the inhibition of bacterial movement by calcium was applicable to several bacterial species. This effect was likely driven by changes in the biophysical properties of the sodium alginate mimetic caused by calcium. Thus, treatment of mucus mimetic (4% sodium alginate) with calcium formulations comprised of calcium and sodium salts reduced the movement of bacterial pathogens through the mimetic.

To extend these studies to non-bacterial pathogens, the effect of calcium was tested on influenza (enveloped virus, about 130 nm) and rhinovirus (non-enveloped virus; about 30 nm) movement using the same system. Treatment of the mimetic with 0.12 M CaCl₂/0.15 M NaCl; IX (Formulation V) or a more concentrated formulation with 10 times the amount of CaCl₂ (1.2 M CaCl₂/0.15 M NaCl; 10×) reduced the concentration of influenza virus compared to the control in a dose-responsive manner. Similar effects were observed when studies were performed with rhinovirus, which is approximately 30 nm in size. Treatment with 0.12 M CaCl₂/0.15 M NaCl; 1× (Formulation V) had no effect on rhinovirus titer in the basolateral buffer over a 180 minute time course, however, when higher concentrations of calcium were delivered using more concentrated solutions viral titers were reduced by 2.4 and 2.3 logio TCID₅₀/mL (FIG. 9).

The data showed that apical treatment of sodium alginate mucus mimetic with calcium containing formulations significantly reduce bacterial and viral movement across the mimetic in a dose responsive manner. The dose of calcium required to block pathogen ingress was directly related to the size of the pathogen: Larger pathogens (bacteria) were slowed by low concentrations of calcium and smaller pathogens (viruses) required higher doses of calcium.

The effect of calcium on the movement of Der p 1, an allergen from house dust mite (HDM), across sodium alginate mimetic was tested. The allergen is one of those used in the human aeroallergen study described in Example 5. Der p 1 possesses protease activity that is believed to act on airway epithelium in a manner that promotes allergic inflammatory response, particularly in asthmatics and populations sensitive to the allergen. The protein is about 25 kD in size and is orders of magnitude smaller than bacterial or viral pathogens.

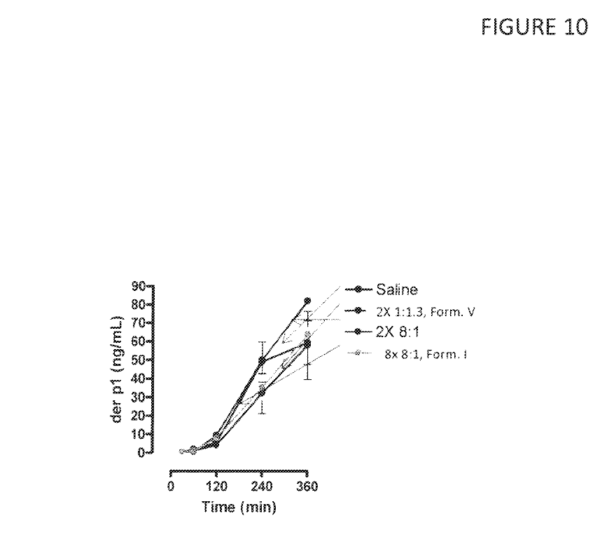

Liquid formulations [0.15 M NaCl (Saline), 0.12 M CaCl₂ in 0.15 M NaCl; 2× tonicity; 1:1.3 ratio of Ca:Na) (Formulation V), 0.21 M CaCl₂ in 0.03 M NaCl (2× tonicity; 8:1 ratio of Ca:Na), or 0.85 M CaCl₂ in 0.11 M NaCl (8× tonicity; 8:1 ratio of Ca:Na)](Formulation I) were topically delivered to the apical surface of mucus mimetic using the liquid sedimentation cell system. These formulations deliver approximately 7.0 microgram calcium ion per cm², 15 microgram calcium ion per cm², and 38 microgram calcium ion per cm² to the surface of the transwell. Immediately following the delivery of formulations, 10 microliter of HDM extract (Greer Laboratories) containing 58.4 microgram/ml Der p 1 was added to the apical surface. Samples of the basolateral buffer (saline) were collected over time and the concentration of Der p 1 was determined by ELISA (Indoor Biotechnologies). No difference in the rate of Der p 1 movement across the mimetic was observed in the calcium treated conditions compared to the saline control (FIG. 10). The inability of the calcium formulations to block or slow the passage of Der p 1 protein suggested there might be a lower size limit to the inhibitory (barrier) effects of particle movement through mucus mimetics by calcium formulations. The data suggest that as pathogen and/or antigen size decreased, pathogen and/or antigen migration through the mucus mimetic increased.

Based on the in vitro results, it was surprising that a calcium salt formulation, Formulation V (0.12 M CaCl₂ in 0.15 M NaCl), in the human patient asthma challenge study described in Example 5 reduced eosinophil recruitment to the asthmatic airways when such recruitment was triggered by soluble aeroallergen. The biophysical effects of calcium salt-based formulations on airway lining fluid were expected, based on the in vitro results, to limit or delay ingress of aeroallergens to effector signaling cells, such as allergen presenting cells (APC) at the airway surface, in a size-dependent manner. If calcium salt formulations influence physical barrier function as the principle means of preventing aeroallergen induced eosinophilic bronchitis, soluble aeroallergens should only have been minimally affected, as compared with naturally occurring particulate aeroallergens. As can be seen, however, calcium salt formulations reduced the number of eosinophils after allergen challenge in patients (FIG. 7), suggesting anti-inflammatory activities independent from and/or in addition to the barrier effect. The barrier function effects of calcium salt formulations are expected to play a role with respect to particulate aeroallergens and such effects may be cumulative with other calcium salt effects. Thus, calcium salt formulations may be particularly useful in the treatment or prevention of aeroallergen challenge by particulate aeroallergens, and may result in enhanced reduction of eosinophil recruitment.

### Example 7

### The Combination of Calcium and Zanamivir is More Effective at Reducing Influenza Infection than Either Compound Alone

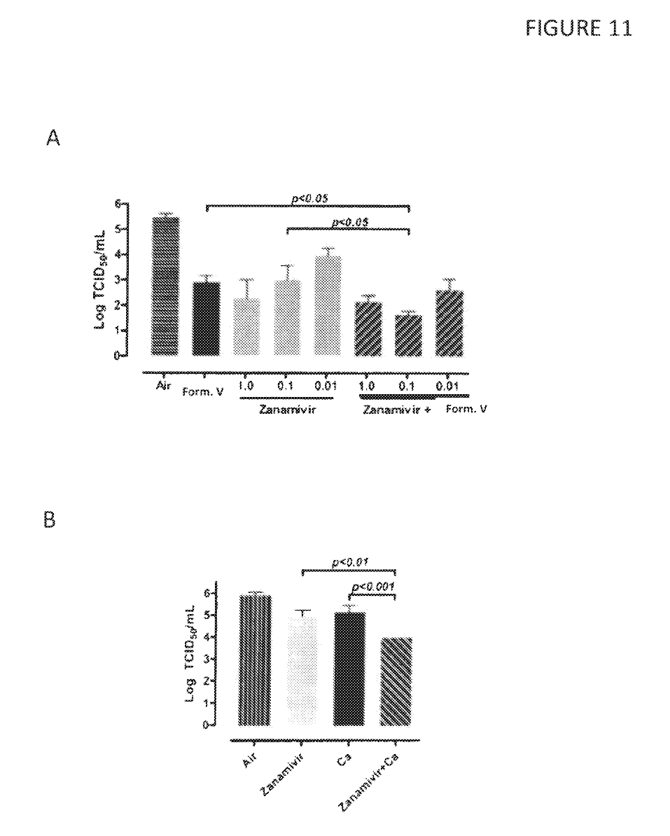

Calu-3 cells were exposed to liquid aerosols of either zanamivir (0.01 to 1.0 nM in PBS) or 1.29% CaCl₂ in 0.9% saline (Formulation V) and infected with Influenza A/WSN/33/1 one hour after exposure. The viral titer on the apical surface of cells was determined 24 hours after dosing. Zanamivir reduced viral infection in a dose responsive manner (p<0.01 compared to untreated (Air) control; one way ANOVA with Tukey's multiple comparison test). Similarly, 1.29% CaCl₂ in 0.9% NaCl (Formulation V) significantly reduced viral titers approximately 300-fold compared to untreated controls, a level that was comparable to the 0.1 nM concentration of zanamivir (FIG. 11A).

To test whether the combination of zanamivir and calcium would further reduce viral infection over zanamivir or 1.29% CaCl₂ in 0.9% NaCl (Formulation V) alone, Calu-3 cells were exposed to the same concentrations of zanamivir in 1.29% CaCl₂ in 0.9% NaCl (Formulation V). The combination formulations each significantly reduced Influenza titers compared to the untreated controls (p<0.001 compared to untreated (Air) control). The combination of Formulation V with 0.01 nM zanamivir resulted in a statistically significant reduction in viral infection compared to the single treatment of Formulation V or the matched zanamivir concentration. The combined treatment effect was ˜20-fold greater (FIG. 11A).

Zanamivir is typically delivered in dry powder form. To determine if the enhanced efficacy of calcium and zanamivir would be evident in dry powder formulations, dry powder formulations were prepared that consisted of zanamivir alone (with NaCl), calcium chloride alone (with NaCl), and the combination of the zanamivir and calcium chloride. The dry powder formulations consisting of either zanamivir alone or calcium chloride alone reduced Influenza titers to similar levels, 8.6- and 5.8-fold respectively. (FIG. 11B) When zanamivir and calcium chloride were co-delivered in the same dry powder formulation, viral titers were further reduced. This reduction was 86-fold compared to the air-control and at least 10-fold greater than either of the single treatments alone. Thus, the combined effects of zanamivir and calcium chloride resulted in enhanced effectiveness in reducing influenza infection in both liquid and dry powder form.

### Example 8

### Calcium-Containing Formulations Reduced Inflammation in an LPS Mouse Model of Acute Lung Injury

In this study, a mouse model of acute lung injury was used to study the effects of Formulation II on pulmonary inflammation. Mice were exposed to aerosolized lipopolysaccharide (LPS) isolated from Pseudomonas aeruginosa. This challenge generally results in lung inflammation and causes changes in pulmonary function. The change in inflammation is marked by an increase in the number of neutrophils in the lungs. Similar changes in lung inflammation and pulmonary function are observed in humans suffering from acute lung injury.

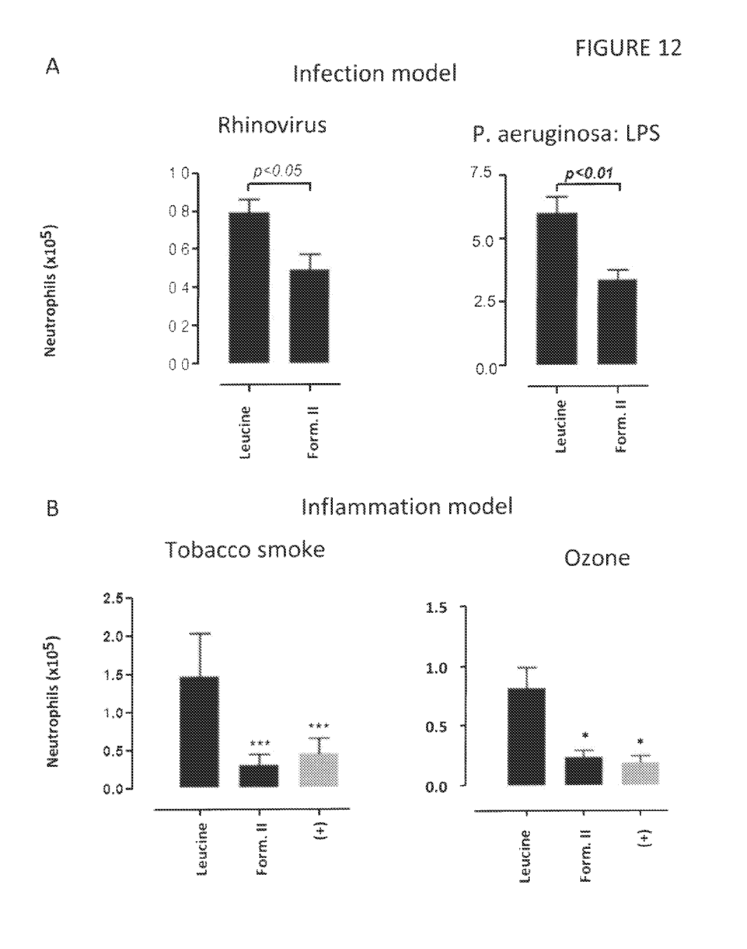

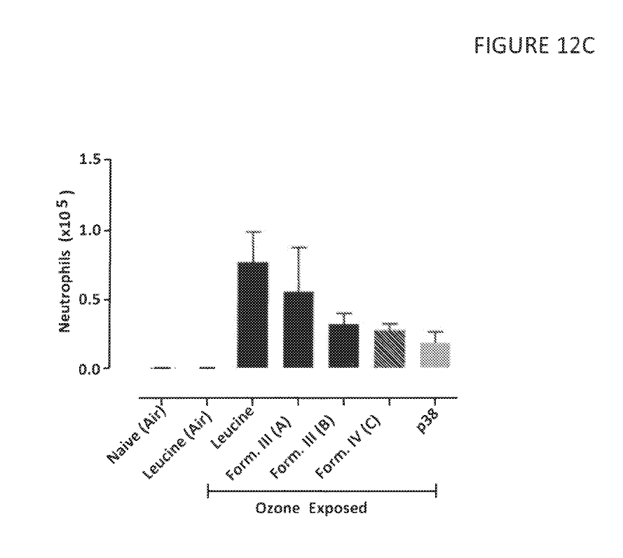

Balb/c mice were challenged with aerosolized LPS Pseudomonas aeruginosa (0.0175 mg/ml) for 20 minutes via a PariLC sprint nebulizer at t=0 hours. Animals were treated with Formulation II at t=−1 hour, +4 hours and +20 hours by aerosol using a whole body exposure system. Control animals were treated with a dry powder placebo (100% leucine). BALs were performed at 24 hours and total and differential cell counts were performed. Data were analyzed by one-way ANOVA and Tukey's multiple comparisons test. Treatment of mice with Formulation II significantly reduced neutrophils count in the BAL fluid when compared with animals exposed to placebo (FIG. 12A, right panel).

### Example 9

### Calcium-Containing Dry Powder Formulations Reduce Neutrophilic Inflammation Following Ozone Exposure

The inflammatory pathway in COPD is thought to be critical to the pathogenesis of the disease and can be studied in animal models of induced pulmonary inflammation, using e.g. endotoxin (lipopolysaccharide, LPS) or exposure to noxious particles (irritants), such as tobacco smoke (TS) and ozone. Ozone-induced pulmonary inflammation is thought to be a good animal model with predictive power for efficacy in chronic obstructive pulmonary disease (COPD). In healthy human subjects, exposure to ozone is known to cause a transient increase in neutrophils and inflammatory mediators recovered in sputum samples and this model has been used to evaluate the anti-inflammatory activity of therapeutic agents, such as CXCR1/2 antagonists, p38 MAP kinase inhibitors and PDE4 inhibitors. Many of the same agents that are tested in the human challenge model have first shown efficacy in preclinical ozone exposure animal models. As pulmonary neutrophilia has been associated with a decline in lung function in patients with COPD, attenuation of neutrophil recruitment and activation may have a positive effect on the disease progression.

Female wild-type (BALB/cJ) were obtained from The Jackson Laboratory (Bar Harbor. ME) at six weeks of age. Mice were treated with dry powder formulations Leucine (98% leucine, 2% sodium chloride) and Formulation II (75% calcium lactate, 20% leucine, 5% sodium chloride) in a temperature and humidity controlled room (30±5% RH and 20±2° C.) or administered liquid treatments of a p38 MAP kinase inhibitor (100 microgram/kg, Tocris SB203580), and USP-Grade Saline (0.9% NaCl), Cardinal Health via the intranasal route. Naïve mice were not exposed to dry powder treatment. One hour following the end of treatment, conscious mice were placed in a custom plastic exposure chamber (26 cm×16 cm×12 cm, Braintree Scientific) on a heated pad at 37° C. Ozone was generated via corona discharge using OZ-2AD Ozone Generator (Ozone Solutions, Hull, Iowa) and diluted with 2.0 LPM (liter per minute) of USP breathing air (MedTech, Medford, Mass.) to a concentration of 3 ppm. The concentration inside the exposure chamber was sampled continuously at 1.0 LPM using UV-106L Ozone Analyzer (Ozone Solutions, Hull, Iowa). Mice were exposed to ozone for 1 hour. Control mice were placed in static cages and exposed to ambient air for a duration matched to each ozone exposure. Mice were euthanized 4 hours following the end of the ozone or air exposure using a fatal dose of pentobarbital. BAL and cell counts were performed. All relevant data sets were combined and presented as Mean±SEM. A Tukey's multiple comparison test was used for statistical comparison of groups, where * denotes a p<0.05. p38 MAP kinase inhibitors (+) have been previously described to reduce the neutrophil influx resulting from ozone exposure. Ozone challenge induced a significant neutrophilic inflammatory response. Formulation II significantly inhibited the influx of neutrophils (FIG. 12B, right panel) with equivalent efficacy of the p38 MAP kinase inhibitor delivered by the intranasal route.

To establish whether other calcium dry powder formulations exhibited similar effects, mice were treated with leucine, Formulation III ((A) exposed dose: 0.8 mg calcium ion/kg animal, (B) exposed dose: 2.3 mg calcium ion/kg animal) and Formulation IV ((C): exposed dose 2.8 mg calcium ion/kg animal) one hour prior to air or ozone exposure. Naïve (untreated) mice were exposed to air. There was no difference in total cell numbers in the BAL between naïve and leucine-treated mice exposed to air; neutrophils were absent in both groups (FIG. 12C). Pulmonary inflammation, indicated by an influx in neutrophils, was evident in the leucine-treated mice exposed to ozone. Mice treated with Formulation III or Formulation IV had a reduction in neutrophils compared to the leucine-treated ozone-exposed animals. A p38 MAP kinase inhibitor significantly inhibited the pulmonary total cell response to ozone exposure. These data suggest that several calcium formulations reduce neutrophilic inflammation following challenge with products of combustion, irritants such as ozone and tobacco smoke.

### Example 10

### Biomarkers for Inflammation, Infection and Irritation

Chronic obstructive pulmonary disease (COPD) is a progressive disease associated with impaired pulmonary function and it primarily occurs as a result of cigarette smoking. COPD subjects are further susceptible to exacerbations that are often associated with an infectious agent and acute inflammation. These exacerbations lead to further declines in lung function, which in turn drives the increased frequency and severity of subsequent exacerbations. Acute exacerbations in asthmatics and COPD patients are a significant cause of lung function decline, morbidity and mortality.

To study both the disease and potential treatments, animal models of COPD have been developed. Animal models of tobacco smoke (TS) exposure have been established to facilitate the testing of novel therapeutics and to evaluate acute airway inflammation following TS exposure (Churg, A. et al. Am J Physiol Lung Cell Mol Physiol 294(4):L612-631, 2008; Churg, A. and J. L. Wright, Proc Am Thorac Soc 6(6):550-552, 2009; Fox, J. C. and Fitzgerald M. R., Curr Opin Pharmacol 9(3):231-242, 2009).

Rhinovirus infection is associated with a significant number of acute exacerbations in COPD and asthma patient populations. Preclinical models of rhinovirus in mice have been hampered by the fact that major strains of rhinovirus do not bind to mouse ICAM-1 and therefore do not infect mouse cells. Recently, a mouse model of rhinovirus infection using a minor strain (RV1B) has been described (Bartlett N W et al. Nat Med. 2008 Feburary: 14(2):199-204). Bartlett et al. describes both rhinovirus infection of naïve mice and rhinovirus infection of ovalbumin-challenged mice as a model of acute exacerbations.

In diseases like allergic asthma and COPD, the influx of inflammatory cells like eosinophils, macrophages and neutrophils into the lung in response to environmental insult is due to cellular release of cytokines and/or chemokines. These cytokines/chemokines signal to induce the chemotaxis of inflammatory cells to the lung. Inhaled irritants such as tobacco smoke activate the release of several growth factors, cytokines and chemokines from airway epithelial cells and macrophages (and other cell types) in the lung, which contribute to the inflammation and tissue damage observed in COPD. These include growth factors such as TFG-beta and FGF, which contribute to fibrosis, and GM-CSF produced by alveolar macrophages, which increases neutrophil and macrophage survival. Pro-inflammatory cytokines amplify inflammation in COPD partly through NFkappaB activation. Tobacco smoke also induces expression of several chemokines which attract circulating cells into the human lungs, e.g. T cells, cosinophils and macrophages.

(1) Using a tobacco smoke (TS) mouse model of COPD, Formulation II was tested for its effects on reducing inflammation and expression of inflammatory cytokines/chemokines. Mice (C57BL6/J) were exposed to TS for up to 45 minutes per day on four successive days by whole body exposure. On each day of TS exposure, mice were treated with Formulation II once daily 1 hour before TS exposure. Formulation II dosing was performed using a whole body exposure system and a capsule based delivery system. A schematic depiction of the study design is shown in Schematic 1.

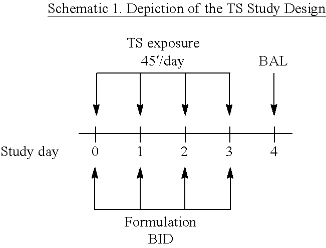

Control animals were exposed to a dry powder formulation of 100% leucine (placebo A) and TS, a second control group was treated with leucine, but not exposed to TS, and a third control group was treated with Formulation II, but not exposed to TS. As a positive control, mice were administered a p38 MAPK inhibitor (+ctrl; 100 microgram/kg) intranasally once a day (ADS 110836, see WO 2009/098612 “POLYMORPHIC FORM OF A [1, 2, 4] TRIAZOLO [4, 3-A] PYRIDINE DERIVATIVE FOR TREATING INFLAMMATORY DISEASES”, Example 11). Neutrophil chemotaxis to the lung was analyzed in these animals for leucine control, p38 control (+), and Formulation II. The data show that Formulation II significantly reduced infiltration of inflammation-associated neutrophils (FIG. 12B, left panel).

(2) Using the naïve mouse model, the efficacy of Formulation II against rhinovirus infection and inflammation was evaluated and cytokine/chemokine protein and gene expression was analyzed. On the day of intranasal infection with Rv1B (5.0×10⁶ TCID₅₀/50 microliter/mouse) BALB/c mice were treated with Formulation II 1 h before and 4 h after infection. One group of animals was exposed to a dry powder formulation of 98% leucine and 2% sodium chloride (placebo B) and not exposed to rhinovirus; one group was treated with Formulation II and not exposed to rhinovirus; one group was treated with placebo B and exposed to rhinovirus; one group was treated with Formulation II and exposed to rhinovirus. Neutrophil chemotaxis to the lung was analyzed in these animals for leucine control and Formulation II. The data show that Formulation II significantly reduced infiltration of inflammation-associated neutrophils (FIG. 12A, left panel).

Lung tissue samples from both tobacco smoke (1) and rhinovirus (2) models were collected and inflammatory biomarkers were determined by gene expression array. For tobacco smoke the samples were collected 4 hours after the final tobacco exposure, for rhinovirus the samples were collected 6 hours after infection. Quantitative polymerase chain reaction (qPCR) arrays (CFX384 Touch Real-Time PCR System, Biorad, Hercules, Calif.)) covering 336 genes of interest were analyzed (with some genes being present on more than one array).

Four focused arrays (SABiosciences RT² Profiler PCR arrays) were used: 1) mouse chemokine and receptor genes (SABiosciences PAMM-022E-4), 2) innate and adaptive immune response genes (SABiosciences PAMM-052E-4), 3) cAMP/Ca²⁺ signaling pathway related genes (SABiosciences PAMM-066E-4), and 4) signal transduction pathway related genes (SABiosciences PAMM-014E-4). Quantification is based on SybrGreen I technology (RT² SYBR Green qPCR Master Mix, Qiagen) using sets of validated primer pairs. mRNA samples were extracted from homogenized frozen mouse lung tissue and converted to complimentary DNA (cDNA) using reverse transcription (RT) reaction. qPCR reactions were performed on 32 samples total (16 mice per study, 4 mice per group), in a 384-well format (4 lung samples and 84 genes per plate), using the following cycles: 1 cycle (10 minutes, 95° C.), 40 cycles (15 seconds, 95° C. followed by 1 minute, 60° C.). The melt curve was automatically generated by CFX Manager 2.0 software): 65° C., 5 seconds (OPTICS OFF); 65° C. to 95° C. at 0.5° C. per minute (OPTICS ON).

The threshold cycle (Ct) for each well was calculated using the CFX Manager 2.0 software. The baseline value is set automatically by this software. The threshold value was manually defined by using the Log View of the amplification plots and placing it above the background signal but within the lower one-third to lower one half of the linear phase of the amplification plot. Web-based statistical software provided by SABiosciences (on the World Wide Web at sabiosciences.com/pcrarraydataanalysis.php) was used to analyze the data (delta/delta ct) ΔΔct obtained for the different treatment groups.

Five housekeeping genes that were unaffected by the experimental conditions were selected as normalization factors. The housekeeping genes were: GUSB. HPRT, HSP90AB1, GAPDH, and ACTB. Data were calculated and expressed as fold change in gene regulation from control group (placebo A or placebo B, respectively) not exposed to either tobacco smoke or rhinovirus.

The following calculations were automatically processed by the NanoDrop™ Software.

Absorbance=−log(Intensityₛₐₘₚₗₑ/Intensity_{blank}) (1)

Nucleic Acid concentrations: Beer-Lambert Equation: c=(A*e)/b, wherein c is the nucleic acid concentration in ng/microliter, A is the absorbance in AU, e is the wavelength-dependent extinction coefficient (40 ng-cm/microliter for RNA), b is the path length in cm (2)

The SABiosciences PCR Array Data Analysis Web Portal utility automatically:
converts all Ct values greater than 35 or as N/A (not detected) to 35. A Ct value of 35 was considered a negative call
calculates Ct values for the Genomic DNA Control. If the value is greater than 35, then the level of genomic DNA contamination does not affect gene expression profiling results
calculates Ct values for the Reverse Transcription Control (RTC)
calculates ΔCt=Avg Ct RTC−Avg Ct PPC, if the value is less than 5, no inhibition is apparent
calculates Ct values for the Positive PCR Control (PPC). The average Ct PPC value should be 20±2 on each PCR Array and should not vary by more than two cycles between PCR Arrays being compared
calculates the ΔCt=Ct GOI−Ct avg_{HKG} for each pathway focused gene on the plate, where GOI is Gene of Interest and HKG is Housekeeping Gene
when biological and/or technical replicates are performed, calculates the average ΔCt value of each gene (each well) across those replicate arrays for each treatment group
calculates the AAct (delta/delta ct) for each gene across two PCR Arrays (or groups). ΔΔct=Δct (group 1)−Δct (group 2), where group 1 is the control and group 2 is the experimental
calculates the fold-change for each gene from group 1 to group 2 as 2^{(ΔΔct)}.

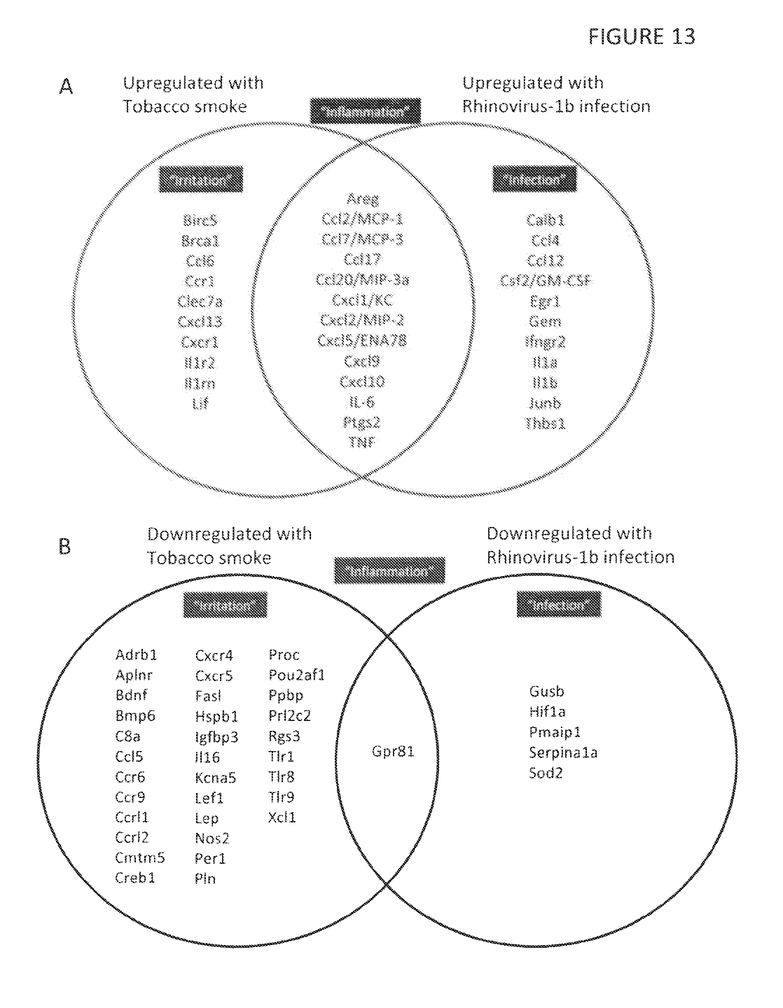

Gene array analysis revealed two distinct biomarker gene signatures, one each for the tobacco smoke and the rhinovirus model. The genes representing the signature were upregulated or downregulated at least two-fold over control. These signatures represent inflammation/irritation and inflammation/infection signatures and the biomarkers cluster into three groups, see FIGS. 13A and 13B. The “irritation” signature (tobacco smoke) and the “infection” signature (rhinovirus) show significant overlap in a group of inflammatory biomarkers (“inflammation” signature). The group common to both the “irritation” signature and the “infection” signature contains several known markers of neutrophil-associated inflammation. The inflammation signature group consists of genes that are upregulated (>2-fold): Areg, Ccl2/MCP-1, Ccl7/MCP-3, Ccl17, Ccl20/MIP-3a, Cxcl1/KC, Cxcl2/MIP-2, Cxcl5/ENA78, Cxcl9, Cxcl10, IL-6, Ptgs2, and TNF, as well as one gene that is downregulated (>2-fold): Gpr81.

Both signatures also contain biomarker genes that are unique to the respective signature. Biomarkers unique for the irritation signature that are upregulated (>2-fold) are: Birc5, Brca1, Ccl6, Ccr1, Clec7a, Cxcl13, Cxcr1, Il1r2, Il1rn, and Lif. Biomarkers unique for the irritation signature that are downregulated (>2-fold) are: Adrb1, Aplnr. Bdnf, Bmp6, C8a, Ccl5, Ccr6, Ccr9, Ccrl1, Ccrl2, Cmtm5, Creb1, Cxcr4, Cxcr5, Fas1, Hspb1, Igfbp3, Il16, Kcna5, Lef1, Lep, Nos2, Per1, Pin, Proc, Pou2af1, Ppbp, Prl2c2, Rgs3, Tlr1, Tlr8, Tlr9, and Xcl1.

Biomarkers unique for the infection signature that are upregulated (>2-fold) are: Calb1, Ccl4, Ccl12, Csf2/GM-CSF, Egr1, Gem, Ifngr2, Il1a, Il1b, Junb, and Thbs1.

Biomarkers unique for the infection signature that are downregulated (>2-fold) are: Gusb, Hif1a, Pmaip1, Serpina1a, and Sod2.

The upregulated and downregulated biomarker genes for the inflammation signature, irritation signature, and infection signature are summarized in Table 5.

TABLE 5
Biomarker Gene Signatures.

| | | |
|---|---|---|
| Inflammation Biomarker | Irritation Biomarker | Infection Biomarker |
| Gene Signature | Gene Signature | Gene Signature |

UPREGULATED GENES > 2-fold

| | | |
|---|---|---|
| Areg (Amphiregulin) | Birc5 (baculoviral IAP repeat- | Calb1 (calbindin 1) |
| | containing 5) | |
| Ccl2/MCP-1 (Chemokine (C- | Brca1 (breast cancer type 1 | Ccl4 (Chemokine (C-C |
| C motif) ligand 2/Monocyte | susceptibility protein) | motif) ligand 4) |
| chemotactic protein-1) | | |
| Ccl7/MCP-3 (Chemokine (C- | Ccl6 (Chemokine (C-C motif) | Ccl12 (Chemokine (C-C |
| C motif) ligand 3/Monocyte | ligand 6) | motif) ligand 12) |
| chemotactic protein-3 | | |
| Ccl17/TARC (Chemokine (C- | Ccr1 (C-C chemokine receptor | Csf2/GM-CSF (colony |
| C motif) ligand 17/thymus and | type 1) | stimulating factor |
| activation regulated | | 2/Granulocyte-macrophage |
| chemokine) | | colony-stimulating factor) |
| Ccl20/MIP-3 alpha | Clec7a/Dectin (C-type lectin | Egr1 (Early growth response |
| (Chemokine (C-C motif) | domain family 7 member A) | protein 1) |
| ligand 20/Macrophage | | |
| inflammatory protein 3-alpha) | | |
| Cxcl1/KC (Chemokine (C-X- | Cxcl13/BLC (Chemokine (C- | Gem (GTP-binding protein) |
| C motif) ligand | X-C motif) ligand 13/B | |
| 1/Keratinocyte-derived protein | lymphocyte chemoattractant) | |
| chemokine) | | |
| Cxcl2/MIP-2 (Chemokine (C- | Cxcr1/IL8RA (chemokine (C- | Ifngr2 (interferon gamma |
| X-C motif) ligand | X-C motif) receptor | receptor 2) |
| 2/Macrophage inflammatory | 1/Interleukin 8 receptor alpha) | |
| protein 2) | | |
| Cxcl5/ENA78 (Chemokine | Il1r2 (Interleukin 1 receptor, | Il1a (Interleukin-1 alpha) |
| (C-X-C motif) ligand 5/ | type II) | |
| epithelial-derived neutrophil- | | |
| activating peptide 78) | | |
| Cxcl9/MIG (Chemokine (C- | Il1rn (interleukin-1 receptor | Il1b (Interleukin 1 beta) |
| X-C motif) ligand 9/ | antagonist) | |
| Monokine induced by gamma | | |
| interferon) | | |
| Cxcl10/IP-10 (Chemokine (C- | Lif (Leukemia inhibitory | JunB (proto-oncogene) |
| X-C motif) ligand | factor) | |
| 10/Interferon gamma-induced | | |
| protein 10) | | |
| Il-6 (Interleukin 6) | | Thbs1 (thrombospondin 1) |
| Ptgs-2/COX-2 (Prostaglandin- | | |
| endoperoxide synthase 2/ | | |
| cyclooxygenase-2) | | |
| TNF-alpha (Tumor necrosis | | |
| factor alpha) | | |

DOWNREGULATED GENES > 2-fold

| | | |
|---|---|---|
| Gpr81(G protein-coupled | Adrb1 (adrenergic receptor | Gusb (Glucuronidase beta) |
| receptor 81) | beta-1) | |
| | Aplnr (apelin receptor) | Hif1a (Hypoxia-inducible |
| | | factor 1 alpha) |
| | Bdnf (Brain-derived | Pmaip1 (phorbol-12- |
| | neurotrophic factor) | myristate-13 -acetate-induced |
| | | protein 1) |
| | Bmp6 (Bone morphogenetic | Serpina1a |
| | protein 6) | |
| | C8a (complement component | Sod2 (Superoxide dismutase |
| | 8 alpha) | 2) |
| | Ccl5/RANTES (Chemokine | |
| | (C-C motif) ligand 5/ | |
| | Regulated upon Activation, | |
| | Normal T-cell Expressed, and | |
| | Secreted) | |
| | Ccr6 (C-C chemokine receptor | |
| | type 6) | |
| | Ccr9 (C-C chemokine receptor | |
| | type 9) | |
| | Ccrl1 (chemokine (C-C motif) | |
| | receptor-like 1) | |
| | Ccrl2 (chemokine (C-C motif) | |
| | receptor-like 2) | |
| | Cmtm5 (CKLF-like | |
| | MARVEL transmembrane | |
| | domain containing 5) | |
| | Creb1 (CAMP responsive | |
| | element binding protein 1) | |
| | Cxcr4/fusin (C-X-C | |
| | chemokine receptor type 4) | |
| | Cxcr5/BLR1 (C-X-C | |
| | chemokine receptor type 5/ | |
| | Burkitt lymphoma receptor 1) | |
| | FasL (Fas ligand) | |
| | Hspb1/Hsp27 (heat shock | |
| | protein beta-1/Heat shock | |
| | protein 27) | |
| | Igfbp3 (Insulin-like growth | |
| | factor-binding protein 3) | |
| | Il16 (interleukin 16) | |
| | Kcna5/Kv1.5 (potassium | |
| | voltage-gated channel, shaker- | |
| | related subfamily, member 5) | |
| | Lef1 (lymphoid enhancer- | |
| | binding factor 1) | |
| | Lep (leptin) | |
| | Nos2 (nitric oxide synthase 2) | |
| | Perl (Period circadian protein | |
| | homolog 1) | |
| | Pln (Phospholamban) | |
| | Proc (pyrroline-5-carboxylate | |
| | reductase) | |
| | Pou2af1 (POU class 2 | |
| | associating factor 1) | |
| | Ppbp/Cxcl7 (pro-platelet basic | |
| | protein/chemokine (C-X-C | |
| | motif) ligand 7) | |
| | Prl2c2 (prolactin family 2, | |
| | subfamily c, member 2) | |
| | Rgs3 (regulator of G-protein | |
| | signaling 3) | |
| | Tlr1 (Toll-like receptor family | |
| | 1) | |
| | Tlr8 (Toll-like receptor family | |
| | 8) | |
| | Tlr9 (Toll-like receptor family | |
| | 9) | |
| | Xcl1 (Chemokine (C motif) | |
| | ligand 1) | |

### Example 11

### Calcium-Containing Formulations Reduce Expression of Biomarkers of Inflammation, Infection and Irritation in Animal Models of Irritation and Viral Infection

Many inflammatory biomarkers that are listed in Table 5 are associated with the NFkappaB/MAPK pathways. However, therapeutic approaches targeting individual mediators or receptors of the NFkappaB/MAPK pathways have not been successful. Thorley A J, Tetley T D, Intern. J COPD 2007:2(4) 409-428. For example, corticosteroids offer a broader anti-inflammatory approach but have proven less effective in COPD. Barnes P J J Clin. Invest. Rev. 2008:118(11).

A calcium-containing formulation, Formulation II, was administered to the tobacco smoke and rhinovirus mouse models, as described above. It was found that Formulation II significantly reduced expression of nearly all pro-inflammatory biomarkers common to both irritation (tobacco smoke) and infection (rhinovirus) biomarker signatures, as shown in Table 6. Values are expressed as fold change from non-irritation or non-infection control, respectively (placebo-exposed).

TABLE 6
A calcium-containing formulation reduced gene
expression of inflammatory biomarkers induced
in an irritation and infection model.

| | Irritation | Irritation | Infection | Infection |
|---|---|---|---|---|
| Inflammation | Model: | Model: | Model: | Model: |
| Gene Signature | Control | Form. II | Control | Form. II |
| Areg | 2.6 | 1.8 | 2.0 | 1.6 |
| Ccl2/MCP-1** | 10.7 | 4.9 ^{Δ2} | 4.5 | 3.4 ^{Δ1} |
| Ccl7/MCP-3 | 6.1 | 4.0 ^{Δ2} | 4.9 | 3.8 |
| Ccl17 | 2.3 | 2.1 | 2.9 | 2.4 |
| Ccl20/MIP-3 a* | 4.9 | 2.9 ^{Δ2} | 9.4 | 13.4 ^{Δ1} |
| Cxcl1/KC* | 20.5 | 9.7 ^{Δ3} | 13.3 | 11.2 ^{Δ1} |
| Cxcl2/MIP-2 | 19.9 | 8.6 ^{Δ2} | 11.9 | 8.7 ^{Δ1} |
| Cxcl5/ENA78 | 16.1 | 8.3 ^{Δ2} | 8.4 | 4.9 ^{Δ1} |
| Cxcl9* | 3.0 | 2.6 ^{Δ2} | 2.0 | −2.5 ^{Δ1} |
| Cxcl10 | 4.3 | 2.6 ^{Δ3} | 2.3 | 1.3 |
| Il-6* | 12.5 | 5.2 ^{Δ3} | 6.5 | 3.9 ^{Δ1} |
| Ptgs-2* | 2.0 | 1.8 | 2.1 | 1.3 |
| TNF-alpha* | 2.3 | 1.3 ^{Δ2} | 8.9 | 6.9 ^{Δ1} |
| Gpr81 | −2.9 | −1.7 | −2.0 | −2.9 |

*average of 2 arrays,
**average of 3 arrays
^{Δ} array data validated by independent qPCR (^{Δ1} n = 7, ^{Δ2} n = 7, ^{Δ3} n = 6)

Several irritation-specific and infection-specific biomarkers were modulated by Formulation II as shown in Tables 7 and 8. Values are expressed as fold change from non-irritation or non-infection control, respectively (placebo-exposed).

TABLE 7
A calcium-containing formulation modulated
gene expression of some biomarkers of irritation.

| Irritation | Irritation Model: | Irritation Model: |
|---|---|---|
| Gene Signature | Control | Form. II |
| Birc5 | 7.1 | 3.6 |
| Brca1 | 2.5 | 1.7 |
| Ccl6 | 2.5 | 2.5 |
| Ccr1 | 2.0 | 1.3 |
| Clec7a | 2.0 | 1.6 |
| Cxcl13 | 2.5 | 1.7 |
| Cxcr1 | 6.0 ^{Δ2} | 5.9 |
| Il1r2 | 5.9 ^{Δ2} | 2.3 |
| Il1rn | 2.0 | 1.4 |
| Lif | 2.1 | 1.4 |
| Adrb1 | −2.2 | −2.1 |
| Aplnr | −4.0 | −2.9 ^{Δ1} |
| Bdnf* | −2.2 | −2.1 |
| Bmp6 | −3.4 | −2.3 |
| C8a | −3.3 | −1.3 |
| Ccl5 | −2.2 | −1.5 ^{Δ2} |
| Ccr6 | −2.6 | −2.3 |
| Ccr9 | −2.1 | −2.0 |
| Ccrl1 | −3.4 | −1.6 |
| Ccrl2 | −2.2 | −1.6 |
| Cmtm5 | −2.2 | −1.0 |
| Creb1 | −2.7 | −1.9 |
| Cxcr4* | −2.2 | −1.8 |
| Cxcr5 | −2.8 | −2.3 |
| FasL | −2.2 | −1.7 |
| Hspb1 | −2.3 | −1.9 |
| Igfbp3 | −7.5 | −3.7 ^{Δ1} |
| Il16 | −2.3 | −1.9 |
| Kcna5 | −2.9 | −1.8 |
| Lef1 | −2.1 | −1.7 |
| Lep | −2.3 | 1.0 |
| Nos2 | −2.7 | −2.1 |
| Perl | −2.1 | −2.2 |
| Pln | −2.5 | −1.8 |
| Proc | −2.5 | −1.5 |
| Pou2af1 | −3.3 | −2.5 |
| Ppbp | −2.0 | −2.1 |
| Prl2c2 | −2.1 | −1.9 |
| Rgs3 | −2.2 | −2.0 |
| Tlr1 | −2.3 | −3.0 |
| Tlr8 | −2.0 | −1.6 |
| Tlr9 | −2.6 | −2.1 |
| Xcl1 | −4.6 | 1.1 |

*average of 2 arrays
^{Δ} array data validated by independent qPCR (^{Δ1} n = 4, ^{Δ2} n = 7)

TABLE 8
A calcium-containing formulation modulated
gene expression of some biomarkers of infection.

| Infection | Infection Model: | Infection Model: |
|---|---|---|
| Gene Signature | Control | Form. II |
| Calb1 | 2.0 | 1.1 |
| Ccl4 | 2.6 | 1.3 |
| Ccl12 | 2.9 | 3.7 |
| Csf2/GM-CSF* | 8.3 | 5.3 ^{Δ1} |
| Egr1* | 3.6 | 3.4 ^{Δ1} |
| Gem | 2.5 | 1.5 |
| Ifngr2 | 10.1 | 9.3 |
| Il1a** | 2.6 | 2.6 |
| Il1b | 2.2 | 1.3 |
| Junb | 2.5 | 1.4 |
| Thbs1 | 2.1 | 1.8 |
| Gusb | −6.2 | −1.2 |
| Hif1a | −10.4 | −1.0 |
| Pmaip1 | −4.8 | 1.2 |
| Serpina1a | −2.8 | −4.2 |
| Sod2 | −5.9 | −1.1 |

*average of 2 arrays,
**average of 3 arrays
^{Δ} array data validated by independent qPCR (^{Δ1} n = 4)

Formulation II appears to exert its anti-inflammatory effect at least in part by reducing irritation-mediated and infection-mediated upregulation of gene expression of several major pro-inflammatory markers and by reducing the irritation-mediated and infection-mediated downregulation of anti-inflammatory markers. Thus, calcium-containing formulations, e.g. Formulation II, may be used as anti-inflammatory agents alone or in combination with additional NFkappaB/MAPK/p38 pathway modulators, e.g. for the treatment of inflammation associated with irritation or infection.

### Example 12

### Calcium-Containing Formulations Modulate Gene Expression in Naïve Animal Models

To assess the effects on biomarker expression on lung tissues of animals in the absence of inflammation, irritation or infection, samples from Formulation II-treated animals that were not exposed to TS and were not infected with rhinovirus were analyzed and compared with non-infected or non-TS exposed animals treated with the placebo (placebo A or B, respectively). The modulated genes are summarized in Table 9. Values are expressed as fold change from non-irritation or non-infection control, respectively (placebo-exposed).

TABLE 9
Modulated genes by Formulation II in non-TS exposed C57BL6
mice and non-infected Balb/c mice greater than 1.5 fold.

| | Formulation II | | Formulation II |
|---|---|---|---|
| Gene | (C57BL6) | Gene | (Balb/c) |
| Camp/cathelicidin | 2.2 ^{Δ1} | Ifngr2 | 7.4 |
| Eno2 | 2.1 | TFF2 | 4.1 |
| Chga | 2.1 | Il2 | 3.0 |
| Fos* | 1.9 | Reg3g | 2.6 |
| S100a9 | 1.8 ^{Δ2} | Trem1 | 2.3 |
| S100a8 | 1.8 ^{Δ2} | IL1r1 | 2.1 |
| Il1f9 | 1.7 | Fosb | 1.9 |
| Egr1 | 1.7^{Δ1} | Lta | 1.8 |
| Lalba | 1.6 | Sele | 1.8 |
| Il2ra | 1.6 | Thbs1 | 1.8 |
| Dusp1/MKP1 | 1.5 | Il6 | 1.7 ^{Δ1} |
| | | Egr1 | 1.7 ^{Δ1} |
| | | Greb1 | 1.7 |
| | | Tfrc | 1.6 |
| | | Il13 | 1.6 |
| | | Birc5 | 1.5 |
| | | Cnn1 | 1.5 |
| | | Il4ra | 1.5 |
| | | Ltb4r2 | 1.5 |
| Ccl20/MIP3a* | −6.0 | Serpina1a | −5.1 |
| Hmox1 | −2.5 | Cxcl9* | −3.4 |
| Nrip1 | −2.1 | Cxcl10 | −2.8 |
| Nos2* | −1.8 | Cxcl2 | −2.6 |
| Ccna1 | −1.7 | Cdkn2a | −2.5 |
| C8a | −1.7 | S100a8 | −2.3 |
| Cxcl5 | −1.5 | Cxcl5 | −2.3 |
| | | Cxcl1* | −2.2 |
| | | S100a9 | −2.1 |
| | | Cd14 | −2.0 |
| | | Tnfsf14 | −2.0 |
| | | Cxcr2 | −2.0 |
| | | Ccl2 | −2.0 |
| | | Ccrl1 | −1.9 |
| | | Tnf** | −1.8 |
| | | Ccl4 | −1.9 |
| | | Il1b | −1.9 |
| | | Il1f9 | −1.8 |
| | | Ccl5 | −1.8 |
| | | Csf2** | −1.8 |
| | | Ccl19 | −1.7 |
| | | Fasl | −1.7 |
| | | Il2ra | −1 7 |
| | | Ccl11 | −1.6 |
| | | Chga | −1.6 |
| | | Trem1 | −1.6 |
| | | Csf1 | −1.6 |
| | | Il12rb2 | −1.6 |
| | | Pglyrp3 | −1.6 |
| | | Proc | −1.6 |
| | | Xcl1 | −1.5 |
| | | Cmtm5 | −1.5 |
| | | Tlr2 | −1.5 |

*average of 2 arrays,
**average of 3 arrays
^{Δ} array data validated by independent qPCR (^{Δ1} n = 4, ^{Δ2} n = 7)

Calcium-containing formulations, e.g. Formulation II, appear to selectively upregulate and downregulate certain sets of pro- and anti-inflammatory markers in the absence of an inflammatory event, such as an irritation or infection.

Cathelicidins/Camp comprise a family of mammalian proteins containing a C-terminal cationic antimicrobial (e.g. bacteria, viruses, fungi) domain that becomes active after being freed from the N-terminal cathelin portion of the holoprotein. The mature peptides show a wide spectrum of antimicrobial and other biological activities. Cathelicidin peptide is found in lysosomes of macrophages, epithelial cells, and polymorphonuclear leukocytes (PMNs). The human cathelicidin peptide LL-37 is chemotactic for neutrophils, monocytes, mast cells, and T cells; induces degranulation of mast cells; alters transcriptional responses in macrophages; stimulates wound vascularization and re-epithelialization of healing skin. The porcine PR-39 has also been involved in a variety of processes, including promotion of wound repair, induction of angiogenesis, neutrophils chemotaxis, and inhibition of the phagocyte NADPH oxidase activity, whereas the bovine BMAP-28 induces apoptosis in transformed cell lines and activated lymphocytes and may thus help with clearance of unwanted cells at sites of inflammation. Zanetti M. J Leukocyte Biol. 2004, 75:39-48.

S100A8 and S100A9 (calprotectin) are small calcium-binding proteins that are highly expressed in neutrophil and monocyte cytosol and are found at high levels in the extracellular milieu during inflammatory conditions. S100A8, S100A9, and S100A8/A9 are potent stimulators of neutrophils and evidence suggests that these proteins are involved in neutrophil migration to inflammatory sites. Ryckman C. et al J Immunol 2003, 170:3233-3242. An important functional aspect of secreted S100 proteins is the ability to act in a cytokine-like manner as extracellular ligands for cell surface receptors, thereby activating signaling cascades and triggering cellular responses. S100A8/A9 induced the activation of NF-kB and an increased phosphorylation of p38 and p44/42 MAP kinases (Hermani A. et al. Exp. Cell Res. 2006, 312:184-197) and also act as endogenous Toll-like receptor 4 agonists, suggesting that they act as innate amplifier of processes such as infection, autoimmunity, and cancer (Ehrchen J M, J Leukoc Biol. 2009 86:557-566).

Dusp1/MKP1, Mitogen-activated protein kinase phosphatase (MKP)-1 is a protein phosphatase that regulates the activity of p38 mitogen-activated protein (MAP) kinase and c-Jun amino-terminal kinase (JNK) and, to lesser extent, p42/44 extracellular signal-regulated kinase. MKP-1 expression is induced in response to a range of stimuli, such as cellular stress, cytokines, LPS, and glucocorticoids, in several inflammatory (such as macrophages) and noninflammatory cells. Studies in MKP-1−/− (null) mice show that MKP-1 is a regulating factor suppressing excessive cytokine production and inflammatory response. MAP kinase phosphatases (MKPs) are dual-specific phosphatases (DUSPs) that dephosphorylate tyrosine and threonine residues in MAP kinases and thereby inactivate them (Liu et al., 2007; Boutros et al., 2008). In macrophages, defects in MKP-1 function results in increased and prolonged p38 activation (Zhao et al., 2005; Hammer et al., 2006; Salojin et al., 2006), and MKP-1^{-/-} (null) mice have increased expression of TNF, IL-6, IL-10, COX-2, and macrophage inflammatory protein-1 in response to in vivo LPS challenge (Salojin el al., 2006). Exposure of MKP-1^{-/-} (null) mice to Staphylococcus aureus or Gram-positive bacterial products resulted in elevated cytokine production and inducible nitric-oxide synthase expression, and these mice had increased mortality rate, increased neutrophil infiltration in lungs, and they suffered from more severe organ damage (Wang et al., 2007).

Nuclear receptor interacting protein 140 (RIP140)/NRIP1 is known as a corepressor but also exhibits coactivator function for the nuclear factor kappaB (NFkappaB), a transcriptional regulator of inflammation in multiple tissues. RIP140 functions as a coactivator for the cytokine gene promoter activity which relies on direct protein-protein interaction with the NFkappaB subunit RelA and histone acetylase cAMP-responsive element binding protein (CREB)-binding protein (CBP). RIP140 deficiency specifically impairs the execution of the pro-inflammatory program and equally impaired cytokine gene activation by TLR2, 3 and 4 signaling. Zschiedrich I. et al. Blood 2008, 112.

### Example 13

### Calcium-Containing Formulations Modulate Expression of Several Genes Associated with Inflammation, Infection and/or Irritation

The effects of Formulation II on additional biomarkers of interest were analyzed and some of the biomarkers from the gene array were verified. The biomarkers are summarized in Table 10. Values are expressed as fold change from non-irritation or non-infection control, respectively (placebo-exposed). The modulation of gene expression of MMP12, XCL1, and Serpina1 by Formulation II was statistically significant. Formulation II downregulated the induced expression of MMP12 and upregulated the reduced expression of XCL1, and Serpina1, thereby to a degree counteracting these biomarker responses to an irritation stimulus.

For infection stimulus, Formulation II upregulated expression of at least 3 genes (of the set of genes tested) that were induced in response to rhinovirus infection, including IL1r1 (Interleukin 1 receptor, type I), TFF2 (Trefoil factor family 2, spasmolytic polypeptide) and Reg3g (Regenerating islet-derived protein 3 gamma). Trefoil factors are critically involved in responses to intestinal injury, primarily by their ability to promote epithelial restitution, the rapid spreading and migration of existing epithelial cells following injury. TFF2 is thought to regulate acid production, stabilize the mucin gel layer (by directly interacting with mucin proteins), and promote healing, as supported by recent studies in TFF2-deficient mice. Reg3g is produced e.g. via stimulation of Toll-like receptors (TLRs) by pathogen-associated molecular patterns (PAMPs). Reg3g specifically targets Gram-positive bacteria.

TABLE 10
A calcium-containing formulation modulated gene expression of
several biomarkers tested in an irritation and infection model.

| | Infection | Infection | Irritation | Irritation |
|---|---|---|---|---|
| | Model: | Model: | Model: | Model: |
| Gene | Control | Form. II | Control | Form. II |
| MMP12 | | | 9.4 | 4.9 p ≦ 0.001 |
| COX2 | | | 3.3 | 2.5 |
| Csf2/GMCSF | | | 2.6 | 2.1 |
| EGR1 | | | 1.5 | 2.0 |
| Reg3g | 1.2 | 2.2 | 1.6 | 1.8 |
| TFF2 | 1.7 | 3.1 | 1.4 | 1.2 |
| Junb | | | 1.2 | 1.2 |
| MMP9 | | | 1.0 | 0.6 |
| IL-2 | | | 0.8 | 0.6 |
| IL1r1 | 2.5 | 3.4 | | |
| Cxcr1/IL8r | 0.9 | 0.8 | | |
| XCL1 | | | −2.5 | −1.6 p ≦ 0.05 |
| Serpina1 | | | −3.4 | −2.0 p ≦ 0.05 |
| Wnt2 | 1.3 | −71.3 | | |
| Tlr1 | −1.2 | −3.4 | | |
| Cxcl9 | 1.2 | −2.5 | | |
| Ncam1 | 1.4 | −2.4 | | |

To assess the effects on biomarker expression on lung tissues of animals in the absence of inflammation, irritation or infection, samples from Formulation II-treated animals that were not exposed to TS and were not infected with rhinovirus were analyzed and compared with non-infected or non-TS exposed animals treated with the placebo (placebo A or B, respectively). The modulated genes are summarized in Table 11. Values are expressed as fold change from non-irritation or non-infection control, respectively (placebo-exposed).

TABLE 11
Modulated genes by Formulation II in non-TS
exposed C57BL6 mice and non-infected Balb/c mice.

| | Formulation II | | Formulation II |
|---|---|---|---|
| Gene | (C57BL6) | Gene | (Balb/c) |
| EGR1 | 2.4 | | |
| MMP9 | 1.1 | | |
| Csf2/GMCSF | 1.2 | | |
| Junb | 1.2 | | |
| Reg3g | 1.0 | Reg3g | 2.6 |
| COX2 | 1.0 | | |
| MMP12 | 1.2 | | |
| TFF2 | 0.7 | TFF2 | 4.1 |
| IL-2 | 1.0 | | |
| | | IL1r1 | 2.1 |
| | | Cxcr1/IL8r | 1.1 |
| XCL1 | −1.1 | | |
| Serpina1 | −1.4 | | |

### Example 14

### Calcium-Containing Formulations Reduce Protein Levels of Biomarkers of Inflammation, Infection and Irritation

The BAL samples obtained from tobacco smoke (TS) exposed and non-exposed C57BL6/J mice treated with either placebo or dry powder Formulation II were analyzed further for pro-inflammatory protein markers. Chemokine and cytokine expression in placebo-exposed non-TS exposed animals was compared to placebo-exposed TS-exposed animals. BAL protein expression levels of twenty analytes were determined using Luminex multiplex assays. The fluorescence intensity of each sample was plotted against a standard curve. Mean protein concentration is reported±SEM in pg/mL. Data were analyzed by one-way ANOVA and Tukey's post-test with a 95% confidence interval. Any value of p>0.05 was labeled not significant (n.s.).

Eight of the twenty targets analyzed were significantly increased (p<0.05, one-way ANOVA and Tukey's post-test) by TS-exposure alone as compared to the air control: IL-6, IL-12 (p40), KC, MCP-1, MIP-1 alpha, MIP-2, MIP-3 alpha, and TGF beta 2. Those exhibiting the greatest fold increase included KC (14-fold; p<0.001), MIP-2 (8.3-fold; p<0.001), MIP-1 alpha (7.5-fold; p<0.001), and MCP-1 (7.3-fold; p<0.001). A moderate fold increase was seen in IL-6 (4.9-fold; p<0.001), MIP-3 alpha (2.6-fold; p<0.001), TGF beta 2 (2.4-fold; p<0.001), and IL-12 p40 (2-fold; p<0.01). Five of the twenty targets analyzed were significantly decreased (one-way ANOVA, p<0.05) by TS-exposure as compared to the air control. Only one target, IL-23 p19 exhibited a large fold decrease (decreased 6-fold; p<0.001) whereas other targets were more moderately reduced, including: IL-17F (decreased 2.6-fold; p<0.01), TGF beta 3 (decreased 2.4-fold; p<0.05), IL-10 (decreased 2-fold; p<0.01), and TNF alpha (decreased 1.8-fold; p<0.01). The remaining targets were not significantly altered by TS-exposure, but were within the range of quantitation of the assay: IL-1 beta, GM-CSF, IFN gamma, RANTES, IL-17, and TGF beta 1.

Chemokine and cytokine expression in placebo exposed non-TS exposed animals was compared to Formulation II treated non-TS-exposed animals. Non-TS exposed animals were administered Formulation II or a dry powder placebo to assess the treatment alone on inflammatory mediator levels. Treatment with Formulation II alone did not cause any significant changes in BAL levels of the twenty targets analyzed in this study as compared to the placebo-exposed non-TS exposed control. However, when Formulation II was given as a treatment in TS-exposed mice, significant changes in protein expression were observed compared to the placebo-exposed control group. Five of the eight proteins that were increased in the BAL of TS-exposed mice were significantly reduced by Formulation II treatment (Table 12): IL-6 (2.1-fold, p<0.01 one-way ANOVA and Tukey's post-test), KC (1.6-fold, p<0.05), MCP-1 (2.2-fold, p<0.001), MIP-2 (1.7-fold, p<0.01), and MIP-3 alpha (2.3-fold, p<0.001). One additional protein was also significantly reduced by Formulation II treatment compared to the placebo-exposed control group when analyzed using an unpaired t-test: MIP-1 alpha (1.9-fold, p<0.001). The remaining two proteins that were induced by TS-exposure were not significantly altered by treatment with Formulation II, however, levels of each protein were lower in the Formulation II treated animals compared to control: IL-12 (p40) and TGF beta 2. Of the five targets found to be decreased in the BAL of TS-exposed mice, none were significantly altered by Formulation II treatment after statistical analysis by one-way ANOVA; however, IL-23 p19 showed a significant 2.5-fold increase with Formulation II over the control group when analyzed by an unpaired t-test.

Table 12 shows chemokine and cytokine expression of eight analytes significantly increased by TS-exposure. BAL protein expression levels of eight analytes shown to increase significantly with TS-exposure and the corresponding levels in Formulation II or p38 MAPK inhibitor treated animals are shown.

TABLE 12
Formulation II reduces TS-exposure induced
chemokine and cytokine protein expression.

| | Leu (TS) | Formulation II | +Ctrl (TS) |
|---|---|---|---|
| Analyte | (pg/ml) | (TS) (pg/ml) | (pg/ml) |
| IL-6 | 30.49*** ± 5.80 | 14.44** ± 2.06 | 18.23 ± 3.59 |
| IL-12 | 313.8** ± 21.71 | 212.7**^{†} ± 19.25 | 298.7 ± 41.21 |
| (p40) | | | |
| KC | 180.7*** ± 18.14 | 115.9* ± 18.69 | 107.3** ± 16.93 |
| MCP-1 | 236.3*** ± 25.72 | 109.0*** ± 9.56 | 188.9 ± 32.99 |
| MIP-1 | 37.60*** ± 3.85 | 19.90***^{†} ± 1.08 | 39.03 ± 9.90 |
| alpha | | | |
| MIP-2 | 68.79*** ± 6.47 | 40.33** ± 4.74 | 50.85 ± 10.05 |
| MIP-3 | 258.6*** ± 24.91 | 110.8*** ± 11.85 | 206.2 ± 31.68 |
| alpha | | | |
| TGF | 188.4*** ± 27.21 | 123.9 ± 19.39 | 160.1 ± 16.07 |
| beta 2 | | | |

ANOVA/Tukey's:
*p < 0.05,
**p < 0.01,
***p < 0.001;
unpaired t-test:
**^{†}p < 0.01,
***^{†}p < 0.001;
Leu (TS) statistics relative to Leu Control, Formulation II (TS) and +Ctrl (TS) statistics relative to Leu (TS)

Treatment of TS-exposed mice with p38 MAPK inhibitor significantly altered protein expression of only one analyte altered by TS-exposure. Of the eight analytes significantly increased in TS-exposed mice, treatment with p38 MAPK inhibitor decreased expression of seven of them but only KC was significantly inhibited by 1.7-fold compared to placebo-exposed control mice by one-way ANOVA (Table 12). Treatment with p38 MAPK inhibitor did not prevent the decrease of any of the five analytes found to be decreased in the BAL of TS-exposed mice.

These data, suggest the mechanism of Formulation II is distinct from that of the p38 MAPK inhibitor in that Formulation II has a more potent impact on protein expression of TS-regulated analytes. Further, the differences in mechanism suggest that calcium-containing formulations may be combined with suitable modulators of inflammatory responses to treat inflammatory responses, e.g. those associated with irritation or infections.

### Example 15

### Calcium Chloride Treatment Reduced Cytokine and Chemokine Secretion in LPS Stimulated Peritoneal Macrophages

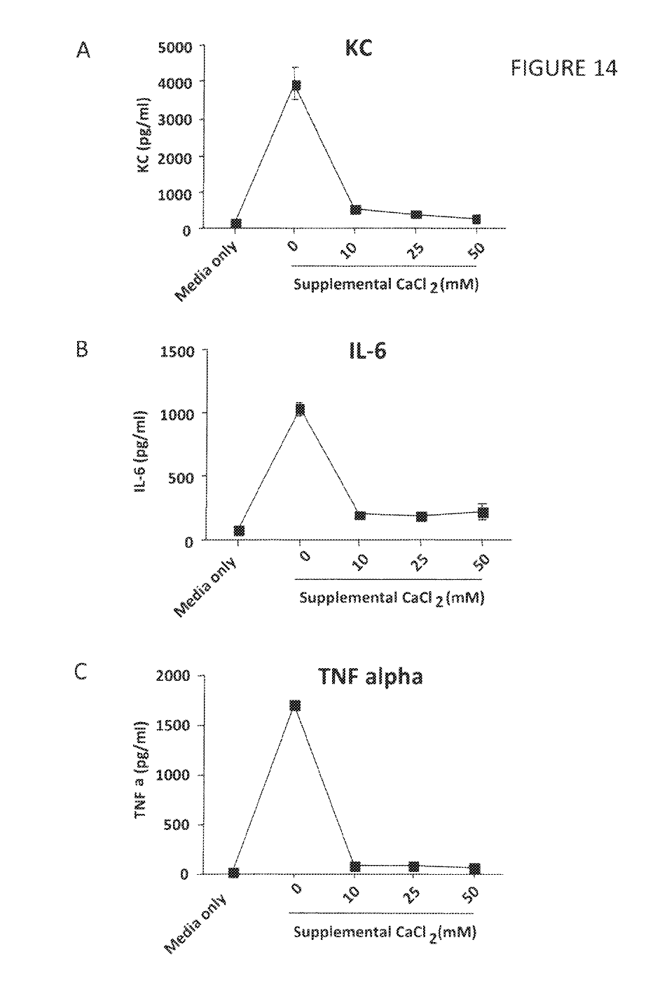

Macrophages represent a key component of the innate immune system. Alveolar macrophages are thought to play a central role in disease pathogenesis by secreting pro-inflammatory cytokines and chemokines in the context of both chronic obstructive pulmonary disease (COPD) and cystic fibrosis (CF), two respiratory illnesses involving airway inflammation. It was hypothesized that given the prominent role of alveolar macrophages in LPS induced lung inflammation, it may be possible that Ca²⁺-ion containing formulations directly influence the inflammatory response of the macrophage after LPS exposure. In order to test this hypothesis in-vitro, murine peritoneal macrophages were isolated. C57BL/6 female mice were anesthetized using isofluorane inhalant. Animals were injected i.p. with 1.5 ml thioglycollate. Three to four days post injection mice were euthanized using CO₂ to harvest peritoneal macrophages. Under sterile conditions, the abdominal wall was exposed and the peritoneal cavity was injected with 7 ml of lavage media, which was immediately recovered back into the syringe and deposited into a 50 ml conical tube. The peritoneal cavity was opened and the residual lavage media was recovered. Cell suspensions were centrifuged at 1200 rpm (RT-6000) for 8-10 minutes. The supernatant was removed and cell pellets were resuspended in 1-2 ml lysis buffer, incubated at room temperature (RT) for 1-2 minutes, and resuspended in 18-19 ml cell culture media (DMEM/F-12). Cells were again centrifuged, supernatants were removed, the cell pellets were pooled, washed once more in cell culture media, centrifuged, and resuspended in 10 ml cell culture media, and cells were counted using a hemocytometer. To confirm macrophage phenotype, a cytospin preparation was performed. Cells were cultured in DMEM:F12 overnight. The following day the media was removed and the macrophages were exposed to media supplemented with varying concentrations of LPS (1 ng/ml to 1 microgram/ml), a dose range of supplemental calcium chloride (5 mM to 50 mM) as well as a dose range of calcium chloride in the presence of LPS for 2 hours. After 2 hours in culture the media was collected for analysis of inflammatory cytokines using LUMINEX® Multiplex immunoassays (Invitrogen/Life Technologies, Grand Island, N.Y.) and BIO-PLEX MANAGER™ software (Version 6) (Bio-Rad, Hercules, Calif.). Presto blue or trypan blue assays were used to determine cell viability. Calcium chloride and/or LPS treatment generally did not affect cell viability. The media content for 3 representative inflammatory mediators (IL-6, KC and TNF-alpha) cultured under LPS free conditions (unstimulated) as well as cells exposed to 1 ng/ml LPS were analyzed. Macrophages that were not exposed to LPS or low levels of supplemental calcium generally had a cytokine contents at or around the limit of detection. Exposure of unstimulated cells to supplemental calcium chloride resulted in some mild increase in inflammatory mediator release with increasing calcium chloride content. When cells were stimulated with LPS, the presence of calcium chloride significantly reduced the protein secretion of KC (FIG. 14A), IL-6 (FIG. 14B), and TNF alpha (FIG. 14C) compared to untreated (“0”) cells. Thus, calcium chloride supplementation to the media resulted in a reduction in pro-inflammatory cytokine secretion during LPS induced inflammation in peritoneal macrophages.

To determine whether calcium chloride treatment influences RNA expression, cells were harvested from naïve mice and allowed to adhere to 12-well plates overnight. This allowed culture of sufficient cells for RNA isolation and qPCR. The macrophages were then stimulated with 1 ng/ml LPS and exposed to increasing concentrations of calcium chloride (0, 10, 25, and 50 mM). Following 2 hours of incubation, cells were lysed, RNA was isolated, and a cDNA reaction was performed. RNA was harvested using the QIAGEN (Germantown, Md.) RNeasy® Plus Mini kit according to the manufacturer's Animal Cell Protocol (2005). RNA concentration and purity was determined by spectrophotometry using NANODROP 1000 spectrophotometer (Thermo Fisher Scientific, Wilmington, Del.). The expression of KC, IL-6, and TNF alpha were assessed by quantitative PCR. RNA expression levels of selected genes were quantified using the delta/delta C(t) (ΔΔCₜ) method using the expression of 18S as an internal reference for each sample. RNA was quantified by a two-step procedure in which RNA was converted to cDNA by reverse transcription using the iSCRIPT cDNA Synthesis kit from Bio-Rad followed by quantitative PCR (qPCR) using iQ SYBR Green Supermix from Bio-Rad.

The following cDNA synthesis reaction conditions were used:

Master Mix:
450-1000 ng of total RNA
add nuclease-free water up to 15 microliter
4 microliter 5× iSCRIPT Reaction Mix
1 microliter iSCRIPT Reverse Transcriptase (or 1 microliter nuclease-free water for control)
Amplification conditions for the RT-PCR reactions were as follows: 25° C. for 5 minutes, 42° C. for 30 minutes, 85° C. for 5 minutes, 25° C. for 5 minutes, 4° C.

The following quantitative PCR reaction conditions were used:

Master Mix:
4 microliter nuclease-free water
0.5 microliter 10 micromolar Forward Primer
0.5 microliter 10 micromolar Reverse Primer
10 microliter 2×SYBR Green 1 RT-PCR Reaction Mix
5.0 microliter cDNA template (1 microliter of 20 microliter cDNA preparation, diluted 1:5 in nuclease-free water).

Amplification conditions for the qPCR reactions were as follows: 95° C. for 3 minutes followed by 40 cycles of 95° C. for 10 seconds, 55° C. for 30 seconds, then a melt curve analysis beginning with 95° C. for 10 seconds, then incubating from 65° C. to 95° C. increasing each by 0.5° C. every 5 seconds.

Data were collected and analyzed using the (delta/delta) ΔΔCₜ method. For each RNA sample the Ct value for 18S was subtracted from the Ct value of the gene of interest (dCt_{target}-dCT₁₈ₛ). The resulting value for the control group (untreated) was then subtracted from the value obtained for each experimental condition (dCTₑₓₑᵣᵢₘₑₙₜₐₗ−dCt_{control}). The fold change was then determined by the equation: 2^{-ΔΔCt}. Statistical analysis was conducted using one-way ANOVA and a Tukey's post-test.

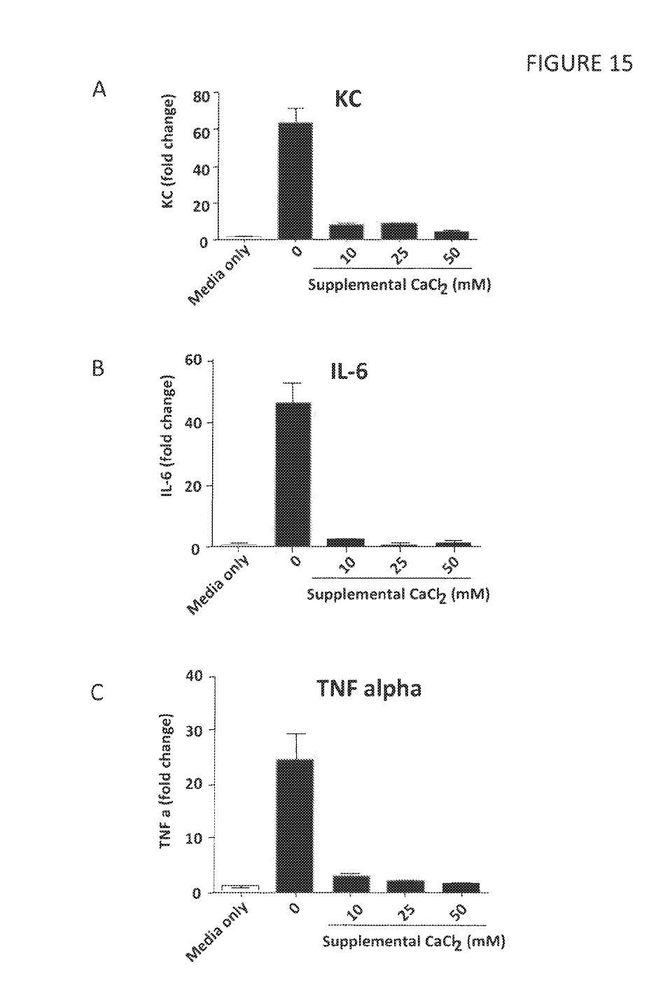

When cells were stimulated with LPS, the presence of calcium chloride significantly reduced gene expression of KC (FIG. 15A), IL-6 (FIG. 15B), and TNFα (FIG. 15C) compared to untreated (“0”). Each data point is mean+SEM of at least three determinations. Analysis of protein secretion and gene expression demonstrated a down regulation of both protein secretion and gene expression in peritoneal macrophages upon treatment with calcium chloride.

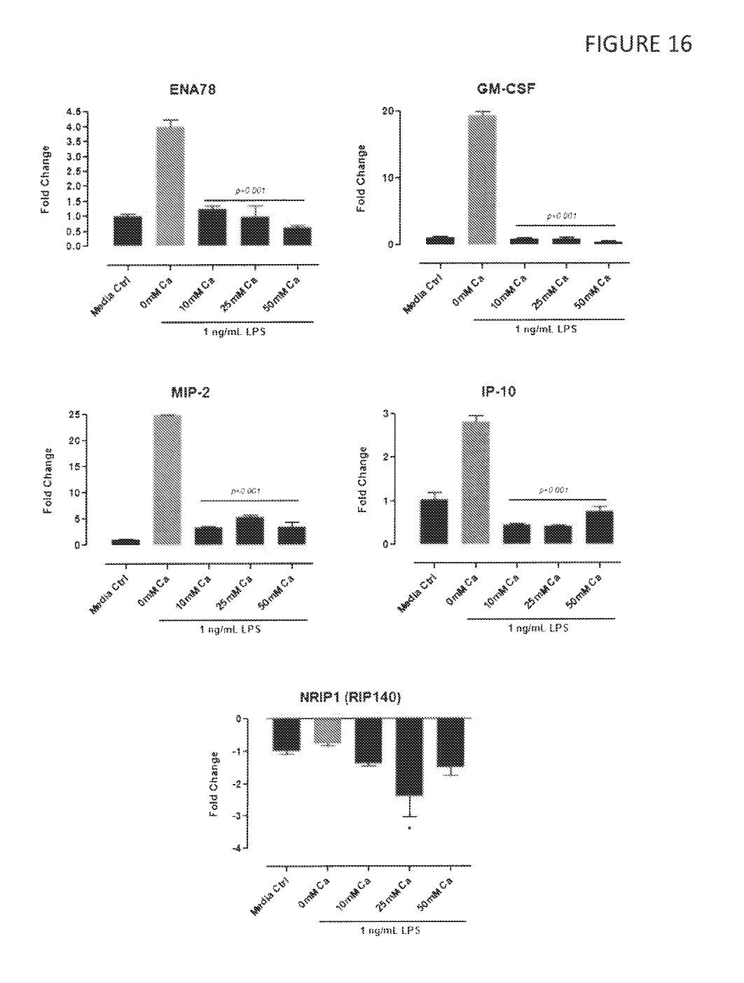

Quantitative PCR was additionally performed for the following targets: ENA78 (Epithelial neutrophil activating protein 78), GM-CSF (Granulocyte-macrophage colony stimulating factor), MIP-2 (Macrophage inflammatory protein-2), IP-10 (interferon gamma-induced protein 10, CXCL10), and NRIP1 (Nuclear receptor-interacting protein 1, RIP140). The targets were selected because of their role as pro-inflammatory mediators associated with macrophage signaling. FIG. 16 shows that LPS induced a three-fold increase in IP-10, a four-fold increase in ENA78 expression, and an approximate 20-fold increase in GM-CSF and MIP-2. When cells were stimulated with LPS, the presence of calcium chloride significantly reduced gene expression of IP-10, ENA78, GM-CSF, and MIP-2 compared to untreated (“0”) cells. A slight induction is evident for NRIP1 upon LPS stimulation (“0”) which is significantly reduced by calcium chloride. Graphs are representative of expression of each respective target as a fold change relative to the “Media Control” group.

### Example 16

### Calcium Chloride Treatment Reduced Cytokine and Chemokine Secretion in Human Macrophages Isolated from Healthy Normal Blood

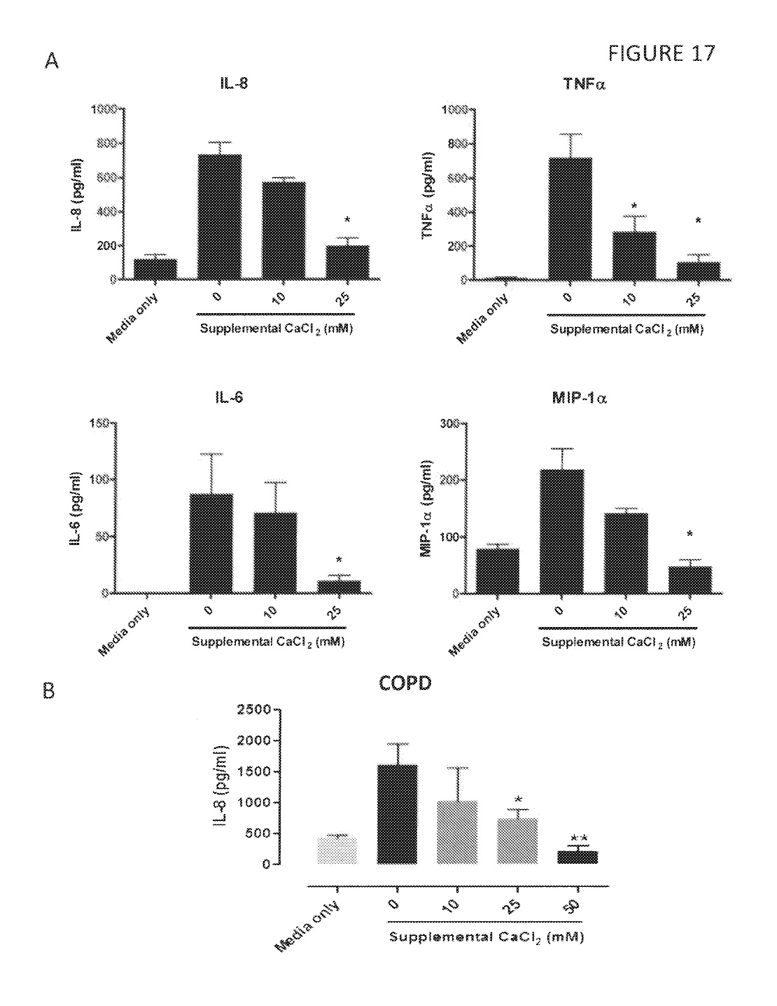

Normal and COPD-derived human monocytes were differentiated into macrophage derived monocytes in culture for 7 days. Cells were thawed following storage in liquid nitrogen, and then plated at 2.0-2.5×10⁵ cells/well (in 0.2 ml per well) in IMDM cell culture media containing 10% human serum and human GM-CSF (50 ng/ml). On day 7, assays were performed on the cells, using media containing serum but no GM-CSF. Cells were stimulated with 10 ng/ml LPS, and exposed to calcium chloride concentrations of either 10 or 25 mM. Following two hours, supernatants were collected for LUMINEX analysis of IL-8, IL-6, TNF alpha and MIP-1 alpha (FIG. 17A), IL-8 only for COPD (FIG. 17B). In each case addition of 25 mM calcium chloride resulted in a significant decline in inflammatory cytokine secretion to near that of non-LPS challenged cells. Calcium chloride reduced LPS induced inflammation in normal and COPD-derived human monocyte-derived macrophages.

### Example 17

### Sodium Chloride Treatment has Little Effect on Cytokine and Chemokine Protein Secretion in Mouse Peritoneal Macrophages and Human Macrophages

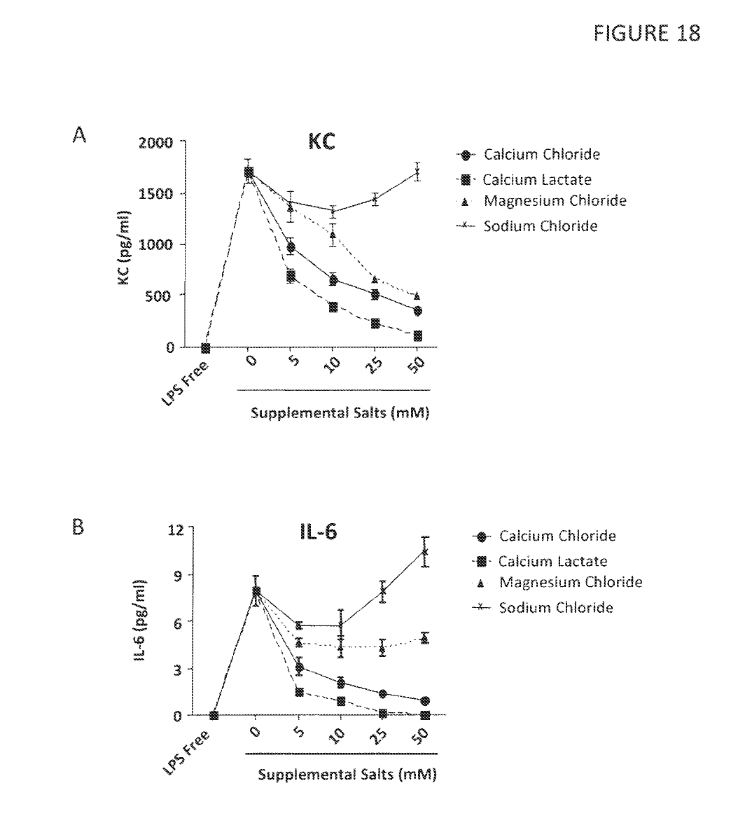

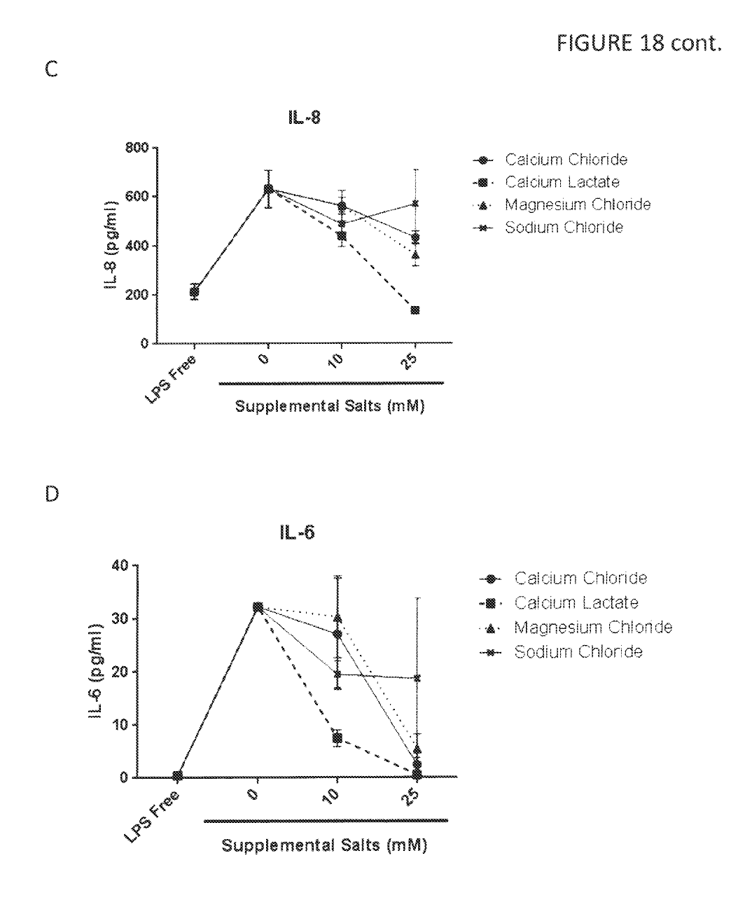

To determine the effects of alternating the cation in the calcium chloride salt, LPS stimulated human or mouse macrophages were exposed to alternative divalent and monovalent cations, magnesium chloride and sodium chloride, as well as an alternative chloride anion, calcium lactate. Normal human monocytes were differentiated into macrophage derived monocytes in culture for 7 days as described in Example 16, and perotineal derived mouse macrophages were described in Example 15. Following two hours of incubation with LPS (10 ng/ml) and the appropriate salt concentration, supernatants were collected for LUMINEX protein analysis of inflammatory cytokines. In mouse macrophages (FIGS. 18A and 18B), calcium chloride resulted in decreased inflammatory mediator secretion. Interestingly, calcium lactate had a more profound effect at reducing LPS induced cytokine production than calcium chloride, particularly KC (FIG. 18A). Magnesium chloride at higher doses demonstrated some activity in reducing inflammatory cytokines, but not to the magnitude of either form of calcium treatment. Sodium chloride had little influence on inflammatory mediator release. In human macrophages (FIGS. 18C and 18D) calcium lactate again exhibited the most profound effect at reducing LPS induced cytokine production in both IL-8 (which is the human functional equivalent to mouse KC) (FIG. 18C) and IL-6 (FIG. 18D). As was the case in mouse macrophages, sodium chloride exhibited little influence on inflammatory mediator release of human macrophages. The effects of calcium chloride and magnesium chloride were of lesser magnitude than calcium lactate but more potent than sodium chloride. Calcium formulations resulted in decreased inflammatory mediator secretion in both mouse and human macrophages and this effect may be specific to divalent cation salts, e.g. calcium and magnesium, and not to monovalent (sodium) salts.

### Example 18

### Calcium Chloride Alters the Inflammatory Response in Peritoneal Macrophages Stimulated with an Array of Toll-Like Receptor (TLR) Ligands

The data in Example 15 showed that inhaled calcium reduced the inflammatory response in mice challenged with LPS. It was hypothesized that calcium likely alters the immune response to LPS and possibly other TLR receptors in alveolar macrophages. To test this hypothesis, primary macrophages were isolated from mice. Briefly, C57BL/6 female mice were anesthetized using isofluorane inhalant. Animals were injected i.p. with 1.5 ml thioglycollate. Three to four days post injection mice were euthanized using CO₂ to harvest peritoneal macrophages. Under sterile conditions, the abdominal wall was exposed and the peritoneal cavity was injected with 7 ml of lavage media, which was immediately recovered back into the syringe and deposited into a 50 mL conical tube. The peritoneal cavity was opened and the residual lavage media was recovered. Cell suspensions were centrifuged at 1200 rpm (RT-6000) for 8-10 minutes. The supernatant was removed and cell pellets were resuspended in 1-2 ml lysis buffer, incubated at room temperature (RT) for 1-2 minutes, and resuspended in18-19 ml cell culture media (DMEM/F-12). Cells were again centrifuged, supernatants were removed, the cell pellets were pooled, washed once more in cell culture media, centrifuged, and resuspended in 10 ml cell culture media, and cells were counted using a hemocytometer. To confirm macrophage phenotype, a cytospin preparation was performed. Cells were cultured in DMEM:F12 overnight. Cells were then stimulated with agonists to TLR4 (LPS or LipidA), TLR1/2 (pam₃CSK₄), TLR2/6 (FLS-1), TLR2 (LT-SA), TLR3 (Poly I:C), TLR 5 (Flagellin), TLR7 (Gardiquimod or Loxoribine) and TLR9 (CPG-ODN) ligands with 0, 10 or 25 mM calcium chloride supplemented to the media. The data in Example 15 demonstrated that changes in KC, the mouse analog of human IL-8, are reflective of trends across most inflammatory cytokines. The magnitude increase in KC content in the media as well as the percent reduction (positive values indicate reduced KC while negative values indicate increased KC content) at 10 and 25 mM calcium chloride are shown in Table 13. Stimulation by all TLR ligands resulted in increased inflammatory mediator secretion to varying degrees, while the changes as a result of calcium supplementation varied significantly. Calcium significantly reduced KC secretion in cells stimulated by TLR 2, 3, 4 and 5. KC secretion in cells stimulated by TLR 1/2, 2/6, 7 and 9 increased with calcium chloride. This data suggests that while calcium can have a marked anti-inflammatory effect in macrophages stimulated by some TLR surface receptors it has a stimulatory effect on others. This data further demonstrates the capacity of calcium and calcium salts to downregulate signaling via some TLR receptors, while simultaneously upregulating the response to others during TLR ligand challenge. Calcium thus modulates TLR signaling over a broad range of TLR receptors.

TABLE 13
KC reduction in TLR-ligand stimulated,
calcium chloride-treated macrophages.

| | % KC reduction at 10 mM | % KC reduction at 25 mM |
|---|---|---|
| TLR | calcium ion | calcium ion |
| 2 | 46 ± 10 | 85 ± 9 |
| 3 | 81 ± 7 | 76 ± 16 |
| 4 | 68 ± 15 | 75 ± 13 |
| 5 | 54 ± 6 | 50 ± 2 |
| 1/2 | −1 ± 9 | −27 ± 58 |
| 2/6 | −25 ± 11 | −51 ± 27 |
| 7 | −10 ± 41 | −20 ± 49 |
| 9 | −55 ± 51 | −144 |

### Example 19

### Calcium Reduces Inflammatory Response Triggered by Gram Negative K. pneumoniae Challenge that Stimulates Primarily TLR4

The data in Example 15 showed that calcium was efficacious in reducing LPS induced inflammation in mouse peritoneal macrophages. The anti-inflammatory properties of calcium during gram positive (S. pneumoniae) and gram negative (Klebsiella pneumoniae) challenge in macrophages was tested.

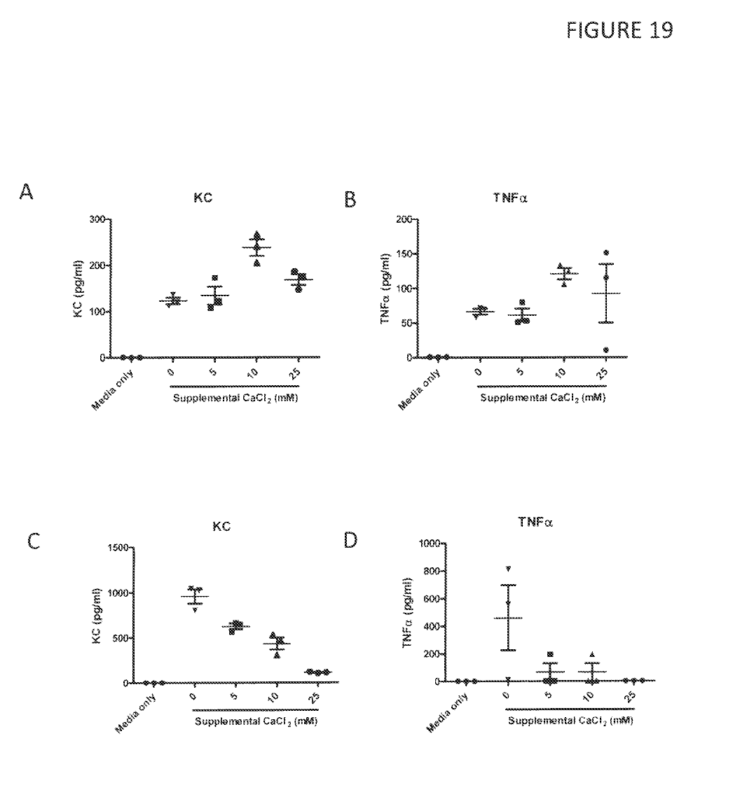

To assess the level of bacterial induced inflammation in isolated peritoneal murine macrophages, isolated cells were placed in cell culture media and allowed to adhere to 96 well plates overnight. Macrophages were then exposed to 1×10; CFU/ml S. pneumonia (FIG. 19A), or 1×10⁷ CFU/ml K. pneumoniae (FIG. 19B) and treated with increasing concentrations 0, 5, 10, or 25 mM calcium chloride. Following two hours of incubation, supernatants were collected for LUMINEX analysis of KC and TNF alpha with the results shown in FIG. 19. In these studies K. pneumoniae challenge results in about a log higher secretion in KC and TNF alpha when compared to stimulation by S. pneumoniae. Treatment with calcium chloride had little effect on inflammatory mediator secretion in macrophages stimulated with S. pneumoniae, while cells stimulated with K. pneumoniae had a dose-dependent decrease in inflammatory mediator secretion with increasing concentrations of calcium chloride. The data in Example 18 demonstrated that macrophage stimulation by TLR3, -4, and -5 was reduced by calcium treatment, whereas inflammation from stimulation by TLR2, -6, and -9 was either unaffected or stimulated by calcium. Given that S. pneumoniae is a gram positive bacteria that stimulates primarily TLR2 and -9, while the gram negative K. pneumoniae stimulates primarily TLR4 these results would seem to corroborate the findings for the TLR ligands in Example 18.

### Example 20

### Influence of Transient Receptor Potential V (TRPV) Channels on the Effect of Calcium

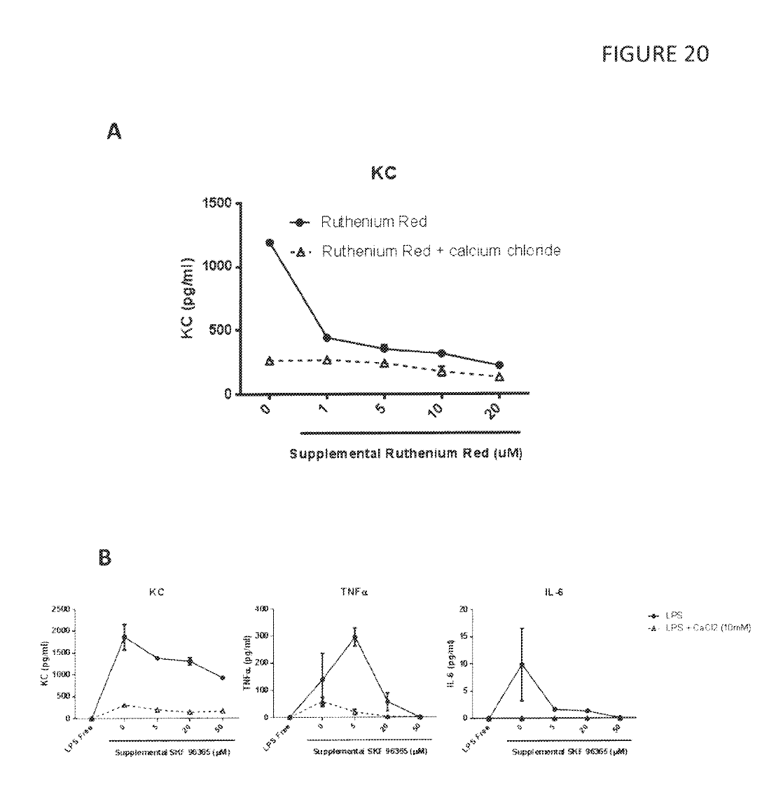

The transient receptor potential V (TRPV) family of ion channels is sensitive to osmotic changes and has an intimate relationship with levels of intracellular calcium. These channels represent a potential target for the reduction in inflammation as a result of calcium chloride treatment. In order to determine the influence of TRPV channels Ruthenium red, a potent, broad acting TRPV channel antagonist was used. Isolated cells were placed in cell culture media and allowed to adhere to 96 well plates overnight. The macrophages were then exposed to Ing/ml of LPS and cells were treated with increasing concentrations of ruthenium red (1, 5, 10 and 20 micromolar) with and without 10 mM calcium chloride. Following two hours of incubation supernatants were collected for LUMINEX analysis of inflammatory cytokines. The results can be seen in FIG. 20A. It can be seen that the supplementation of the media with ruthenium red alone resulted in a decrease in KC concentration by more than 50% and this was further enhanced in the presence of 10 mM calcium chloride. The observation that both calcium and ruthenium red result in a similar reduction in KC secretion suggests that TRPV channels could play a role in calcium's effect on inflammation in macrophages. This further suggests that ruthenium red and calcium chloride given alone or together could further reduce LPS induced inflammation.

To determine the involvement of TRPV2 channels in the TRPV associated inflammation reduction in LPS stimulated macrophages, LPS stimulated (10 ng/ml) peritoneal macrophages were exposed to increasing concentrations of SKF 96365 (5, 20, and 50 micromolar), a non-specific TRPV2 antagonist, with and without 10 mM calcium chloride supplemented into the media. Following two hours, supernatants were collected for LUMINEX analysis of inflammatory cytokines. KC, TNF alpha and IL-6 concentrations from the supernatants are shown in FIG. 20B. Addition of SKF 96365 resulted in a dose dependent decrease in both KC and IL-6. TNF alpha was also reduced at the highest two doses (20 and 50 micromolar) of SKF 96365. However, while treatment with SKF96365 did have an effect on LPS stimulated macrophages, it was not as pronounced as that of treatment with ruthenium red. The highest dose of SKF 96365 (50 micromolar) resulted in only a 51% decrease in KC concentration. Addition of SKF 96365 along with 10 mM calcium chloride resulted in a small increase in KC and TNF alpha reduction when compared to treatment with calcium chloride alone. Taken together, these data suggest that TRPV2 may play a role in calcium mediated reduction of LPS stimulated inflammatory mediator release from macrophages.

What is claimed is:

1-9. (canceled)

10. A method for treating an inflammatory and/or infectious respiratory disease, comprising administering to the lungs of a subject in need of such treatment a respirable calcium salt formulation in an amount of about 0.005 mg Ca²⁺ ion/kg bodyweight to about 0.2 mg Ca²⁺ ion/kg bodyweight, wherein the amount of calcium ion is effective in reducing the inflammation and/or reducing the severity of infection thereby treating the inflammatory and/or infectious respiratory disease.

11. The method of claim 10, wherein the inflammation is chronic.

12. The method of claim 10, wherein the infection is acute.

13. The method of claim 10, wherein the infection is a viral or bacterial infection.

14. The method of claim 10, wherein the subject exhibits an acute exacerbation.

15. The method of claim 10, wherein the respiratory disease is COPD or asthma.

16. The method of claim 10, the method further comprising administering an anti-inflammatory and/or anti-infectious agent.

17. A method for treating an inflammatory and/or infectious respiratory disease, comprising administering to the lung of a subject in need of such treatment a respirable calcium salt formulation in an amount of
i) less than about 0.2 mg Ca²⁺/kg bodyweight,
ii) less than about 0.1 mg Ca²⁺/kg bodyweight, or
iii) less than about 0.005 mg Ca²⁺/kg bodyweight; and
an anti-inflammatory and/or anti-infectious agent in an amount effective to treat the inflammatory and/or infectious respiratory disease.

18-20. (canceled)

21. The method of claim 17, wherein the respiratory disease is associated with excess airway mucus, mucus hypersecretion and/or impaired mucociliary clearance.

22. The method of claim 17, wherein the method further comprises administering a mucociliary clearance (MCC) augmenting agent.

23. The method of claim 22, wherein the MCC augmenting agent is selected from the group consisting of mannitol, hypertonic saline, an epithelial sodium channel (ENaC) blocker, a channel-activating protease inhibitor, a P2Y₂-receptor agonist, ATP, UTP, SABA, LABA, and leucine.

24. The method of claim 17, wherein the calcium salt formulation is administered prior to administration of the anti-inflammatory, anti-infectious or MCC-augmenting agent.

25. The method of claim 17, wherein the calcium salt formulation is administered concurrent with administration of the anti-inflammatory, anti-infectious and/or MCC-augmenting agent.

26. The method of claim 10, wherein the calcium salt formulation is administered as an aerosolized liquid formulation or a dry powder.

27. The method of claim 26, wherein the amount of calcium ion delivered to the lungs is determined by a method comprising determining the fine particle dose (FPD) of the dry powder or liquid formulation.

28-29. (canceled)

30. The method of claim 10, wherein the calcium salt is calcium lactate.

31. The method of claim 10, wherein the calcium salt formulation further comprises i) a monovalent metal cation salt, ii) a pharmaceutically acceptable excipient, and/or iii) a therapeutic agent.

32. The method of claim 31, wherein the monovalent metal cation salt is a sodium salt.

33. The method of claim 31, wherein the therapeutic agent is selected from the group consisting of a mucoactive or mucolytic agent, a surfactant, an antibiotic, an antiviral, an antihistamine, a cough suppressant, a bronchodilator, an anti-inflammatory agent, a steroid, a vaccine, an adjuvant, an expectorant, an antifibrotic agent, and a macromolecule.

34. The method of claim 17, wherein the respiratory diseases is asthma, airway hyper-responsiveness, seasonal allergic allergy, bronchiectasis, chronic bronchitis, emphysema, chronic obstructive pulmonary disease, idiopathic pulmonary fibrosis or cystic fibrosis.

35-151. (canceled)
